(19) United States
(12) Patent Application Publication
(10) Pub. No.: US 2010/0105150 A1
(43) Pub. Date: Apr. 29, 2010

(54) ISOLATED HUMAN AUTOANTIBODIES TO NEUTROPHIL GELATINASE-ASSOCIATED LIPOCALIN (NGAL) AND METHODS AND KITS FOR THE DETECTION OF HUMAN AUTOANTIBODIES TO NGAL

(75) Inventors: Maciej Adamczyk, Gurnee, IL (US); Roy Jeffrey Brashear, Mundelein, IL (US); Phillip G. Mattingly, Third Lake, IL (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(73) Assignee: ABBOTT LABORATORIES, Abbott Park, IL (US)

(21) Appl. No.: 12/257,984

(22) Filed: Oct. 24, 2008

(51) Int. Cl. G01N 33/566; C07K 16/18

(52) U.S. Cl. 436/501; 436/547; 530/387.1

(57) ABSTRACT

A method of determining the presence, amount or concentration of at least one autoantibody that reacts with neutrophil gelatinase-associated lipocalin (NGAL), alone or in further combination with a method of determining the concentration of NGAL, which methods can further comprise diagnosing, prognosticating, or assessing the efficacy of a therapeutic/prophylactic treatment of a patient and, optionally, modifying the therapeutic/prophylactic treatment of the patient as needed to improve efficacy; a kit comprising at least one component for assaying a test sample for at least one autoantibody that reacts with NGAL and instructions for assaying; a method of isolating an autoantibody that reacts with NGAL; an isolated autoantibody that reacts with NGAL; and a method for determining the reliability of an NGAL assay result.

### TECHNICAL FIELD

The disclosure relates to isolated human autoantibodies and related kits and methods for detecting a human autoantibody to NGAL or fragment thereof, and determining the reliability of an NGAL assay result.

### BACKGROUND

Neutrophil gelatinase-associated lipocalin (NGAL), which is also known as human neutrophil lipocalin (HNL), N-formyl peptide binding protein, and 25 kDa α2-microglobulin-related protein, is a 24 kDa protein, which can exist as a monomer, a homodimer, or a heterodimer with proteins, such as gelatinase B or matrix metalloproteinase-9 (MMP-9). NGAL is a marker in the diagnosis and/or prognosis of a number of diseases (see, e.g., Xu et al., Biochim. et Biophys. Acta 1482: 298-307 (2000)), disorders, and conditions, including inflammation, such as that associated with infection. It is a marker for irritable bowel syndrome (see, e.g., U.S. Pat. App. Pub. Nos. 2008/0166719 and 2008/0085524); renal disorders, diseases and injuries (see, e.g., U.S. Pat. App. Pub. Nos. 2008/0090304, 2008/0014644, 2008/0014604, 2007/0254370, and 2007/0037232); systemic inflammatory response syndrome (SIRS), sepsis, severe sepsis, septic shock and multiple organ dysfunction syndrome (MODS) (see, e.g., U.S. Pat. App. Pub. Nos. 2008/0050832 and 2007/0092911; see, also, U.S. Pat. No. 6,136,526); periodontal disease (see, e.g., U.S. Pat. No. 5,866,432); and venous thromboembolic disease (see, e.g., U.S. Pat. App. Pub. Nos. 2007/0269836), among others. In its free, uncomplexed form it is a marker for ovarian cancer, invasive and noninvasive breast cancer, and atypical ductal hyperplasia, which is a major risk factor for breast cancer (see, e.g., U.S. Pat. App. Pub. No. 2007/0196876; see, also, U.S. Pat. Nos. 5,627,034 and 5,846,739 with regard to assessing the proliferative status of a carcinoma). When complexed with MMP-9, it also is a marker for conditions associated with tissue remodeling (see, e.g., U.S. Pat. App. Pub. No. 2007/0105166 and U.S. Pat. No. 7,153,660). A high level of NGAL (e.g., approximately 350 μg/L (Xu et al., Scand. J. Clin. Lab. Invest. 55: 125-131 (1995)) also can be indicative of a bacterial infection as opposed to a viral infection (see, e.g., U.S. Pat. App. Pub. No. 2004/0115728).

Levels of NGAL in the serum of a healthy human range from approximately 50 μg/L to approximately 100 μg/L (Xu et al., J. Immunol. Methods 171: 245-252 (1994); and Blaser et al., Clinica Chimica Acta 235: 137-145 (1995)). Currently available assays for NGAL, such as those used in diagnosis, prognosis, and assessment of therapeutic or prophylactic efficacy, assess the level of NGAL in a test sample, such as a fluid sample, in particular a sample of serum, plasma, or urine, from a patient. Such assays compare the level of NGAL in the test sample to what is considered to be normal, what is considered to be indicative of a given condition, disorder, or disease state, or the level of NGAL in a test sample taken from the patient previously. Based on such comparisons, diagnoses, prognoses, and therapeutic/prophylactic treatment regimens (e.g., active agent used or dosage of a given active agent) are determined. Unfortunately, results obtained with such assays are subject to interferences that may lead to aberrant results, for example, false-negatives, false positives, or incorrect quantification, due to the presence of heretofore unknown endogenous anti-NGAL antibodies.

In view of the foregoing, it is an object of the present disclosure to provide an isolated human autoantibody to NGAL. It is another object of the present disclosure to provide methods and kits for detecting autoantibodies to NGAL in a test sample. Such methods and kits can be used with methods and kits for the detection of NGAL and/or NGAL complexed with another protein so as to minimize aberrant results due to the presence of NGAL autoantibodies in the test sample and thereby improve diagnoses, prognoses and assessments of therapeutic/prophylactic efficacy. These and other objects and advantages, as well as additional inventive features, will become apparent from the detailed description provided herein.

### SUMMARY

A method of determining the presence, amount or concentration of at least one autoantibody that reacts with neutrophil gelatinase-associated lipocalin (NGAL) or a fragment thereof in a test sample is provided. The method comprises assaying the test sample for at least one autoantibody that reacts with NGAL (or a fragment thereof) by an assay employing NGAL (or a fragment thereof) and at least one detectable label and comprising comparing a signal generated by the detectable label as a direct or indirect indication of the presence, amount or concentration of at least one autoantibody that reacts with NGAL (or a fragment thereof) in the test sample to a signal generated as a direct or indirect indication of the presence, amount or concentration of an antibody that reacts with NGAL (or a fragment thereof) in a control or calibrator. The calibrator is optionally part of a series of calibrators in which each of the calibrators differs from the other calibrators in the series by the concentration of an antibody that reacts with NGAL (or a fragment thereof). The method can be adapted for use in an automated system or a semi-automated system.

The method can comprise (i) contacting the test sample with NGAL (or a fragment thereof), which comprises a detectable label and binds to at least one autoantibody to form an NGAL (or fragment thereof)/autoantibody complex, and (ii) determining the presence, amount or concentration of at least one autoantibody, which reacts with NGAL (or a fragment thereof), in the test sample by detecting or measuring the signal generated by the detectable label in the NGAL (or fragment thereof)/autoantibody complex formed in (i).

The method can comprise (i) contacting the test sample with NGAL (or a fragment thereof), which binds to at least one autoantibody and which is optionally immobilized on a solid phase, so as to form an NGAL (or a fragment thereof)/autoantibody complex, (ii) contacting the NGAL (or a fragment thereof)/autoantibody complex with at least one detection antibody, which comprises a detectable label and binds to the autoantibody to form an NGAL (or a fragment thereof)/autoantibody/detection antibody complex, and (iii) determining the presence, amount or concentration of an autoantibody, which reacts with NGAL (or fragment thereof), in the test sample by detecting or measuring the signal generated by the detectable label in the NGAL (or a fragment thereof)/autoantibody/detection antibody complex formed in (ii). The method optionally further comprises removing any unbound at least one autoantibody after step (i) and removing any unbound at least one detection antibody after step (ii).

The method can further comprise previously, simultaneously or subsequently determining the concentration of NGAL (or a fragment thereof) in the test sample. The method can comprise assaying the test sample for NGAL (or a fragment thereof) by an assay employing at least one specific binding partner for NGAL (or a fragment thereof) and at least one detectable label and comprising comparing a signal generated by the detectable label as a direct or indirect indication of the concentration of NGAL (or a fragment thereof) in the test sample to a signal generated as a direct or indirect indication of the concentration of NGAL in a control or calibrator. The calibrator is optionally part of a series of calibrators in which each of the calibrators differs from the other calibrators in the series by the concentration of NGAL.

The method can further comprise diagnosing, prognosticating, or assessing the efficacy of a therapeutic/prophylactic treatment of a patient from whom the test sample was obtained. If the method further comprises assessing the efficacy of a therapeutic/prophylactic treatment of the patient from whom the test sample was obtained, the method optionally further comprises modifying the therapeutic/prophylactic treatment of the patient as needed to improve efficacy.

Also provided is a kit for assaying a test sample for the presence, amount or concentration of at least one autoantibody that reacts with NGAL (or a fragment thereof) in a test sample. The kit comprises at least one component for assaying the test sample for at least one autoantibody that reacts with NGAL (or a fragment thereof) and instructions for assaying the test sample for at least one autoantibody that reacts with NGAL (or a fragment thereof). The at least one component for assaying the test sample for at least one autoantibody that reacts with NGAL (or a fragment thereof) includes a composition comprising NGAL (or a fragment thereof), which is optionally immobilized on a solid phase, and/or a composition comprising an antibody that can bind to the at least one autoantibody that reacts with NGAL (or a fragment thereof), wherein the NGAL (or a fragment thereof) or the antibody is optionally detectably labeled.

Further provided is a method of isolating an autoantibody that reacts with NGAL. The method comprises (i) contacting NGAL (or a fragment thereof) with a biological sample, which is known to contain an autoantibody that reacts with NGAL, wherein the NGAL is optionally immobilized on a solid phase before or after contact with the biological sample, (ii) isolating NGAL to which is bound the autoantibody, and (iii) isolating the autoantibody from the NGAL. Still further provided is an isolated autoantibody that reacts with NGAL.

### DETAILED DESCRIPTION

The present disclosure is based, at least in part, on the surprising and unexpected discovery of endogenous antibodies, i.e., autoantibodies, in human serum and plasma that react with neutrophil gelatinase-associated lipocalin (NGAL).

### Definitions

(a) “Neutrophil gelatinase-associated lipocalin (NGAL),” which is also known as human neutrophil lipocalin (HNL), N-formyl peptide binding protein, and 25 kDa α2-microglobulin-related protein, is a 24 kDa protein, which can exist as a monomer, a homodimer, or a heterodimer with proteins, such as gelatinase B or matrix metalloproteinase-9 (MMP-9). See, e.g., Kjeldsen et al., J. Biol. Chem. 268 (14): 10425-10432 (1993), for an exemplary amino acid sequence. Generally, when present, the signal peptide comprises amino acids 1-20. Numbering as applied herein is considering a signal peptide at residues 1-20, with the understanding that such a signal peptide may or may not be present.

The NGAL polynucleotide or polypeptide can be any NGAL sequence, e.g., including that set forth as Genbank accession numbers Genpept CAA58127 (SEQ ID NO:1), AAB26529, XP_{—}862322, XP_{—}548441, P80108, P11672, X83006.1, X99133.1, CAA67574.1, BC033089.1, AAH33089.1, S75256.1, AD14168.1, JC2339, 1DFVA, 1DFVB, 1L6MA, 1L6MB, 1L6MC, 1NGLA, 1QQSA, 1X71A, 1X71B, 1X71C, 1X89A, 1X89B, 1X89C, 1X8UA, 1X8UB, and 1X8UC. NGAL polynucleotide and polypeptide (e.g., polyamino acid) sequences are as found in nature, based on sequences found in nature, isolated, synthetic, semi-synthetic, recombinant, or other. In one embodiment, the NGAL is human NGAL (also known as “hNGAL”). NGAL polypeptide sequences can be of the mature human NGAL sequence (sequence not including the 20-residue amino acid signal peptide typically found in nature, and/or minus any other signal peptide sequence). When a signal peptide is present, it is numbered, e.g., as residues 1 to 20, with comparable numbering applied for the encoding polynucleotide sequence.

Likewise, an initial Met residue at the N-terminus of NGAL is present only in NGAL produced in prokaryotes (e.g., E. coli), or in synthetic (including semi-synthetic) or derived sequences, and not in NGAL produced in eukaryotes (e.g., mammalian cells, including human and yeast cells). Consequently, when present, an initial Met residue is typically counted as a negative number, e.g., as residue −1, with no similar numbering adjustment being made for the polynucleotide sequence in a prokaryotic versus eukaryotic background or expression system inasmuch as the polynucleotide sequence is replicated and transcribed the same in both backgrounds and the difference lies at the level of translation.

Accordingly, the disclosure herein encompasses a multitude of different NGAL polynucleotide and polypeptide sequences as present and/or produced in a prokaryotic and/or eukaryotic background (e.g., with consequent optimization for codon recognition). In sum, the sequences may or may not possess or encode: (a) a signal peptide; (b) an initiator Met residue present in the mature NGAL sequence at the N-terminus; (c) an initiator Met residue present at the start of a signal peptide that precedes the mature NGAL protein; and (d) other variations such as would be apparent to one skilled in the art.

Exemplary sequences include, but are not limited to, those as set forth herein: SEQ ID NO: 1 (NGAL wild-type polypeptide including signal peptide); SEQ ID NO:2 (NGAL wild-type polypeptide not including any signal peptide, and which can be preceded by a Met initiator residue when produced in prokaryotes and a Met initiator codon is present; however, there is no Met initiator residue when produced in eukaryotes, regardless of whether a Met initiator codon is present); and SEQ ID NO:3 (NGAL wild-type polynucleotide sequence including that encoding a signal peptide). Exemplary sequences further include any mutant sequences set forth in any one or more of U.S. Provisional Application Nos. 60/981,470, 60/981,471 and 60/981,473, all filed on Oct. 19, 2007, and U.S. patent application Ser. Nos. 12/104,408, 12/104,410, and 12/104,413, all filed on Apr. 16, 2008, and each of which are incorporated by reference in their entireties for their teachings regarding same.

Use of “NGAL” herein is intended to encompass NGAL and fragments thereof, unless otherwise contradicted by context.

As used herein, the term “NGAL fragment” herein refers to a polypeptide that comprises a part that is less than the entirety of a mature NGAL (e.g., human NGAL) or NGAL including a signal peptide. In particular, a NGAL fragment comprises from about 5 to about 178 or about 179 contiguous amino acids of SEQ ID NOS:1 or 2. In particular, a NGAL fragment comprises from about 5 to about 170 contiguous amino acids of SEQ ID NOS:1 or 2. In particular, a NGAL fragment comprises at least about 5 contiguous amino acids of SEQ ID NO:1 or 2, at least about 10 contiguous amino acids residues of SEQ ID NOS:1 or 2; at least about 15 contiguous amino acids residues of amino acids of SEQ ID NOS:1 or 2; at least about 20 contiguous amino acids residues of SEQ ID NOS:1 or 2; at least about 25 contiguous amino acids residues of SEQ ID NOS:1 or 2, at least about 30 contiguous amino acid residues of amino acids of SEQ ID NOS:1 or 2, at least about 35 contiguous amino acid residues of SEQ ID NOS:1 or 2, at least about 40 contiguous amino acid residues of SEQ ID NOS:1 or 2, at least about 45 contiguous amino acid residues of SEQ ID NOS:1 or 2, at least about 50 contiguous amino acid residues of SEQ ID NOS:1 or 2, at least about 55 contiguous amino acid residues of SEQ ID NOS:1 or 2, at least about 60 contiguous amino acid residues of SEQ ID NOS:1 or 2, at least about 65 contiguous amino acid residues of SEQ ID NOS:1 or 2, at least about 70 contiguous amino acid residues of SEQ ID NOS:1 or 2, at least about 75 contiguous amino acid residues of SEQ ID NOS:1 or 2, at least about 80 contiguous amino acid residues of SEQ ID NOS:1 or 2, at least about 85 contiguous amino acid residues of SEQ ID NOS:1 or 2, at least about 90 contiguous amino acid residues of SEQ ID NOS:1 or 2, at least about 95 contiguous amino acid residues of SEQ ID NOS:1 or 2, at least about 100 contiguous amino acid residues of SEQ ID NOS:1 or 2, at least about 105 contiguous amino acid residues of SEQ ID NOS:1 or 2, at least about 110 contiguous amino acid residues of SEQ ID NOS:1 or 2, at least about 115 contiguous amino acid residues of SEQ ID NOS:1 or 2, at least about 120 contiguous amino acid residues of SEQ ID NOS:1 or 2, at least about 125 contiguous amino acid residues of SEQ ID NOS:1 or 2, at least about 130 contiguous amino acid residues of SEQ ID NOS:1 or 2, at least about 135 contiguous amino acid residues of SEQ ID NOS:1 or 2, at least about 140 contiguous amino acid residues of SEQ ID NOS:1 or 2, at least about 145 contiguous amino acid residues of SEQ ID NOS:1 or 2, at least about 150 contiguous amino acid residues of SEQ ID NOS:1 or 2, at least about 160 contiguous amino acid residues of SEQ ID NOS:1 or 2, at least about 165 contiguous amino acid residues of SEQ ID NOS:1 or 2, at least about 170 contiguous amino acid residues of SEQ ID NOS:1 or 2 or at least about 175 contiguous amino acid residues of SEQ ID NOS:1 or 2.

A fragment of NGAL contains a contiguous or nonlinear epitope of NGAL. The precise boundaries of such an epitope fragment can be confirmed using ordinary skill in the art. The fragment preferably comprises at least about 5 contiguous amino acids, such as about 10 contiguous amino acids, about 15 contiguous amino acids, or about 20 contiguous amino acids. Examples of such fragments (with reference to the wild-type amino acid sequence) include, but are not limited to, fragments comprising amino acids 21-198, 22-198, 23-198, 24-198, 25-198, 26-198, 27-198, 28-198, 29-198, 30-198, 31-198, 32-198, 33-198, 34-198, 35-198, 36-198, 37-198, 38-198, 39-198, 40-198, 41-198, 42-198, 43-198, 44-198, 45-198, 46-198, 47-198, 48-198, 49-198, 50-198, 51-198, 52-198, 53-198, 54-198, 55-198, 56-198, 57-198, 58-198, 59-198, 60-198, 61-198, 62-198, 63-198, 64-198, 65-198, 66-198, 67-198, 68-198, 69-198, 70-198, 71-198, 72-198, 73-198, 74-198, 75-198, 76-198, 77-198, 78-198, 79-198, 80-198, 81-198, 82-198, 83-198, 84-198, 85-198, 86-198, 87-198, 88-198, 89-198, 90-198, 91-198, 92-198, 93-198, 94-198, 95-198, 96-198, 97-198, 98-198, 99-198, 100-198, 101-198, 102-198, 103-198, 104-198, 105-198, 106-198, 107-198, 108-198, 109-198, 110-198, 111-198, 112-198, 113-198, 114-198, 115-198, 116-198, 117-198, 118-198, 119-198, 120-198, 121-198, 122-198, 123-198, 124-198, 125-198, 126-198, 127-198, 128-198, 129-198, 130-198, 131-198, 132-198, 133-198, 134-198, 135-198, 136-198, 137-198, 138-198, 139-198, 140-198, 141-198, 142-198, 143-198, 144-198, 145-198, 146-198, 147-198, 148-198, 149-198, 150-198, 151-198, 152-198, 153-198, 154-198, 155-198, 156-198, 157-198, 158-198, 159-198, 160-198, 161-198, 162-198, 163-198, 164-198, 165-198, 166-198, 167-198, 168-198, 169-198, 170-198, 171-198, 172-198, 173-198, 174-198, 175-198, 176-198, 177-198, 178-198, 179-198, 180-198, 181-198, 182-198, 183-198, 184-198, 185-198, 186-198, 187-198, 188-198, 189-198, 190-198, 191-198, 192-198, 193-198, 194-198, 21-197, 22-197, 23-197, 24-197, 25-197, 26-197, 27-197, 28-197, 29-197, 30-197, 31-197, 32-197, 33-197, 34-197, 35-197, 36-197, 37-197, 38-197, 39-197, 40-197, 41-197, 42-197, 43-197, 44-197, 45-197, 46-197, 47-197, 48-197, 49-197, 50-197, 51-197, 52-197, 53-197, 54-197, 55-197, 56-197, 57-197, 58-197, 59-197, 60-197, 61-197, 62-197, 63-197, 64-197, 65-197, 66-197, 67-197, 68-197, 69-197, 70-197, 71-197, 72-197, 73-197, 74-197, 75-197, 76-197, 77-197, 78-197, 79-197, 80-197, 81-197, 82-197, 83-197, 84-197, 85-197, 86-197, 87-197, 88-197, 89-197, 90-197, 91-197, 92-197, 93-197, 94-197, 95-197, 96-197, 97-197, 98-197, 99-197, 100-197, 101-197, 102-197, 103-197, 104-197, 105-197, 106-197, 107-197, 108-197, 109-197, 110-197, 111-197, 112-197, 113-197, 114-197, 115-197, 116-197, 117-197, 118-197, 119-197, 120-197, 121-197, 122-197, 123-197, 124-197, 125-197, 126-197, 127-197, 128-197, 129-197, 130-197, 131-197, 132-197, 133-197, 134-197, 135-197, 136-197, 137-197, 138-197, 139-197, 140-197, 141-197, 142-197, 143-197, 144-197, 145-197, 146-197, 147-197, 148-197, 149-197, 150-197, 151-197, 152-197, 153-197, 154-197, 155-197, 156-197, 157-197, 158-197, 159-197, 160-197, 161-197, 162-197, 163-197, 164-197, 165-197, 166-197, 167-197, 168-197, 169-197, 170-197, 171-197, 172-197, 173-197, 174-197, 175-197, 176-197, 177-197, 178-197, 179-197, 180-197, 181-197, 182-197, 183-197, 184-197, 185-197, 186-197, 187-197, 188-197, 189-197, 190-197, 191-197, 192-197, 193-197, 21-196, 22-196, 23-196, 24-196, 25-196, 26-196, 27-196, 28-196, 29-196, 30-196, 31-196, 32-196, 33-196, 34-196, 35-196, 36-196, 37-196, 38-196, 39-196, 40-196, 41-196, 42-196, 43-196, 44-196, 45-196, 46-196, 47-196, 48-196, 49-196, 50-196, 51-196, 52-196, 53-196, 54-196, 55-196, 56-196, 57-196, 58-196, 59-196, 60-196, 61-196, 62-196, 63-196, 64-196, 65-196, 66-196, 67-196, 68-196, 69-196, 70-196, 71-196, 72-196, 73-196, 74-196, 75-196, 76-196, 77-196, 78-196, 79-196, 80-196, 81-196, 82-196, 83-196, 84-196, 85-196, 86-196, 87-196, 88-196, 89-196, 90-196, 91-196, 92-196, 93-196, 94-196, 95-196, 96-196, 97-196, 98-196, 99-196, 100-196, 101-196, 102-196, 103-196, 104-196, 105-196, 106-196, 107-196, 108-196, 109-196, 110-196, 111-196, 112-196, 113-196, 114-196, 115-196, 116-196, 117-196, 118-196, 119-196, 120-196, 121-196, 122-196, 123-196, 124-196, 125-196, 126-196, 127-196, 128-196, 129-196, 130-196, 131-196, 132-196, 133-196, 134-196, 135-196, 136-196, 137-196, 138-196, 139-196, 140-196, 141-196, 142-196, 143-196, 144-196, 145-196, 146-196, 147-196, 148-196, 149-196, 150-196, 151-196, 152-196, 153-196, 154-196, 155-196, 156-196, 157-196, 158-196, 159-196, 160-196, 161-196, 162-196, 163-196, 164-196, 165-196, 166-196, 167-196, 168-196, 169-196, 170-196, 171-196, 172-196, 173-196, 174-196, 175-196, 176-196, 177-196, 178-196, 179-196, 180-196, 181-196, 182-196, 183-196, 184-196, 185-196, 186-196, 187-196, 188-196, 189-196, 190-196, 191-196, 192-196, 21-195, 22-195, 23-195, 24-195, 25-195, 26-195, 27-195, 28-195, 29-195, 30-195, 31-195, 32-195, 33-195, 34-195, 35-195, 36-195, 37-195, 38-195, 39-195, 40-195, 41-195, 42-195, 43-195, 44-195, 45-195, 46-195, 47-195, 48-195, 49-195, 50-195, 51-195, 52-195, 53-195, 54-195, 55-195, 56-195, 57-195, 58-195, 59-195, 60-195, 61-195, 62-195, 63-195, 64-195, 65-195, 66-195, 67-195, 68-195, 69-195, 70-195, 71-195, 72-195, 73-195, 74-195, 75-195, 76-195, 77-195, 78-195, 79-195, 80-195, 81-195, 82-195, 83-195, 84-195, 85-195, 86-195, 87-195, 88-195, 89-195, 90-195, 91-195, 92-195, 93-195, 94-195, 95-195, 96-195, 97-195, 98-195, 99-195, 100-195, 101-195, 102-195, 103-195, 104-195, 105-195, 106-195, 107-195, 108-195, 109-195, 110-195, 111-195, 112-195, 113-195, 114-195, 115-195, 116-195, 117-195, 118-195, 119-195, 120-195, 121-195, 122-195, 123-195, 124-195, 125-195, 126-195, 127-195, 128-195, 129-195, 130-195, 131-195, 132-195, 133-195, 134-195, 135-195, 136-195, 137-195, 138-195, 139-195, 140-195, 141-195, 142-195, 143-195, 144-195, 145-195, 146-195, 147-195, 148-195, 149-195, 150-195, 151-195, 152-195, 153-195, 154-195, 155-195, 156-195, 157-195, 158-195, 159-195, 160-195, 161-195, 162-195, 163-195, 164-195, 165-195, 166-195, 167-195, 168-195, 169-195, 170-195, 171-195, 172-195, 173-195, 174-195, 175-195, 176-195, 177-195, 178-195, 179-195, 180-195, 181-195, 182-195, 183-195, 184-195, 185-195, 186-195, 187-195, 188-195, 189-195, 190-195, 191-195, 21-194, 22-194, 23-194, 24-194, 25-194, 26-194, 27-194, 28-194, 29-194, 30-194, 31-194, 32-194, 33-194, 34-194, 35-194, 36-194, 37-194, 38-194, 39-194, 40-194, 41-194, 42-194, 43-194, 44-194, 45-194, 46-194, 47-194, 48-194, 49-194, 50-194, 51-194, 52-194, 53-194, 54-194, 55-194, 56-194, 57-194, 58-194, 59-194, 60-194, 61-194, 62-194, 63-194, 64-194, 65-194, 66-194, 67-194, 68-194, 69-194, 70-194, 71-194, 72-194, 73-194, 74-194, 75-194, 76-194, 77-194, 78-194, 79-194, 80-194, 81-194, 82-194, 83-194, 84-194, 85-194, 86-194, 87-194, 88-194, 89-194, 90-194, 91-194, 92-194, 93-194, 94-194, 95-194, 96-194, 97-194, 98-194, 99-194, 100-194, 101-194, 102-194, 103-194, 104-194, 105-194, 106-194, 107-194, 108-194, 109-194, 110-194, 111-194, 112-194, 113-194, 114-194, 115-194, 116-194, 117-194, 118-194, 119-194, 120-194, 121-194, 122-194, 123-194, 124-194, 125-194, 126-194, 127-194, 128-194, 129-194, 130-194, 131-194, 132-194, 133-194, 134-194, 135-194, 136-194, 137-194, 138-194, 139-194, 140-194, 141-194, 142-194, 143-194, 144-194, 145-194, 146-194, 147-194, 148-194, 149-194, 150-194, 151-194, 152-194, 153-194, 154-194, 155-194, 156-194, 157-194, 158-194, 159-194, 160-194, 161-194, 162-194, 163-194, 164-194, 165-194, 166-194, 167-194, 168-194, 169-194, 170-194, 171-194, 172-194, 173-194, 174-194, 175-194, 176-194, 177-194, 178-194, 179-194, 180-194, 181-194, 182-194, 183-194, 184-194, 185-194, 186-194, 187-194, 188-194, 189-194, 190-194, 21-193, 22-193, 23-193, 24-193, 25-193, 26-193, 27-193, 28-193, 29-193, 30-193, 31-193, 32-193, 33-193, 34-193, 35-193, 36-193, 37-193, 38-193, 39-193, 40-193, 41-193, 42-193, 43-193, 44-193, 45-193, 46-193, 47-193, 48-193, 49-193, 50-193, 51-193, 52-193, 53-193, 54-193, 55-193, 56-193, 57-193, 58-193, 59-193, 60-193, 61-193, 62-193, 63-193, 64-193, 65-193, 66-193, 67-193, 68-193, 69-193, 70-193, 71-193, 72-193, 73-193, 74-193, 75-193, 76-193, 77-193, 78-193, 79-193, 80-193, 81-193, 82-193, 83-193, 84-193, 85-193, 86-193, 87-193, 88-193, 89-193, 90-193, 91-193, 92-193, 93-193, 94-193, 95-193, 96-193, 97-193, 98-193, 99-193, 100-193, 101-193, 102-193, 103-193, 104-193, 105-193, 106-193, 107-193, 108-193, 109-193, 110-193, 111-193, 112-193, 113-193, 114-193, 115-193, 116-193, 117-193, 118-193, 119-193, 120-193, 121-193, 122-193, 123-193, 124-193, 125-193, 126-193, 127-193, 128-193, 129-193, 130-193, 131-193, 132-193, 133-193, 134-193, 135-193, 136-193, 137-193, 138-193, 139-193, 140-193, 141-193, 142-193, 143-193, 144-193, 145-193, 146-193, 147-193, 148-193, 149-193, 150-193, 151-193, 152-193, 153-193, 154-193, 155-193, 156-193, 157-193, 158-193, 159-193, 160-193, 161-193, 162-193, 163-193, 164-193, 165-193, 166-193, 167-193, 168-193, 169-193, 170-193, 171-193, 172-193, 173-193, 174-193, 175-193, 176-193, 177-193, 178-193, 179-193, 180-193, 181-193, 182-193, 183-193, 184-193, 185-193, 186-193, 187-193, 188-193, 189-193, 21-192, 22-192, 23-192, 24-192, 25-192, 26-192, 27-192, 28-192, 29-192, 30-192, 31-192, 32-192, 33-192, 34-192, 35-192, 36-192, 37-192, 38-192, 39-192, 40-192, 41-192, 42-192, 43-192, 44-192, 45-192, 46-192, 47-192, 48-192, 49-192, 50-192, 51-192, 52-192, 53-192, 54-192, 55-192, 56-192, 57-192, 58-192, 59-192, 60-192, 61-192, 62-192, 63-192, 64-192, 65-192, 66-192, 67-192, 68-192, 69-192, 70-192, 71-192, 72-192, 73-192, 74-192, 75-192, 76-192, 77-192, 78-192, 79-192, 80-192, 81-192, 82-192, 83-192, 84-192, 85-192, 86-192, 87-192, 88-192, 89-192, 90-192, 91-192, 92-192, 93-192, 94-192, 95-192, 96-192, 97-192, 98-192, 99-192, 100-192, 101-192, 102-192, 103-192, 104-192, 105-192, 106-192, 107-192, 108-192, 109-192, 110-192, 111-192, 112-192, 113-192, 114-192, 115-192, 116-192, 117-192, 118-192, 119-192, 120-192, 121-192, 122-192, 123-192, 124-192, 125-192, 126-192, 127-192, 128-192, 129-192, 130-192, 131-192, 132-192, 133-192, 134-192, 135-192, 136-192, 137-192, 138-192, 139-192, 140-192, 141-192, 142-192, 143-192, 144-192, 145-192, 146-192, 147-192, 148-192, 149-192, 150-192, 151-192, 152-192, 153-192, 154-192, 155-192, 156-192, 157-192, 158-192, 159-192, 160-192, 161-192, 162-192, 163-192, 164-192, 165-192, 166-192, 167-192, 168-192, 169-192, 170-192, 171-192, 172-192, 173-192, 174-192, 175-192, 176-192, 177-192, 178-192, 179-192, 180-192, 181-192, 182-192, 183-192, 184-192, 185-192, 186-192, 187-192, 188-192, 21-191, 22-191, 23-191, 24-191, 25-191, 26-191, 27-191, 28-191, 29-191, 30-191, 31-191, 32-191, 33-191, 34-191, 35-191, 36-191, 37-191, 38-191, 39-191, 40-191, 41-191, 42-191, 43-191, 44-191, 45-191, 46-191, 47-191, 48-191, 49-191, 50-191, 51-191, 52-191, 53-191, 54-191, 55-191, 56-191, 57-191, 58-191, 59-191, 60-191, 61-191, 62-191, 63-191, 64-191, 65-191, 66-191, 67-191, 68-191, 69-191, 70-191, 71-191, 72-191, 73-191, 74-191, 75-191, 76-191, 77-191, 78-191, 79-191, 80-191, 81-191, 82-191, 83-191, 84-191, 85-191, 86-191, 87-191, 88-191, 89-191, 90-191, 91-191, 92-191, 93-191, 94-191, 95-191, 96-191, 97-191, 98-191, 99-191, 100-191, 101-191, 102-191, 103-191, 104-191, 105-191, 106-191, 107-191, 108-191, 109-191, 110-191, 111-191, 112-191, 113-191, 114-191, 115-191, 116-191, 117-191, 118-191, 119-191, 120-191, 121-191, 122-191, 123-191, 124-191, 125-191, 126-191, 127-191, 128-191, 129-191, 130-191, 131-191, 132-191, 133-191, 134-191, 135-191, 136-191, 137-191, 138-191, 139-191, 140-191, 141-191, 142-191, 143-191, 144-191, 145-191, 146-191, 147-191, 148-191, 149-191, 150-191, 151-191, 152-191, 153-191, 154-191, 155-191, 156-191, 157-191, 158-191, 159-191, 160-191, 161-191, 162-191, 163-191, 164-191, 165-191, 166-191, 167-191, 168-191, 169-191, 170-191, 171-191, 172-191, 173-191, 174-191, 175-191, 176-191, 177-191, 178-191, 179-191, 180-191, 181-191, 182-191, 183-191, 184-191, 185-191, 186-191, 187-191, 21-190, 22-190, 23-190, 24-190, 25-190, 26-190, 27-190, 28-190, 29-190, 30-190, 31-190, 32-190, 33-190, 34-190, 35-190, 36-190, 37-190, 38-190, 39-190, 40-190, 41-190, 42-190, 43-190, 44-190, 45-190, 46-190, 47-190, 48-190, 49-190, 50-190, 51-190, 52-190, 53-190, 54-190, 55-190, 56-190, 57-190, 58-190, 59-190, 60-190, 61-190, 62-190, 63-190, 64-190, 65-190, 66-190, 67-190, 68-190, 69-190, 70-190, 71-190, 72-190, 73-190, 74-190, 75-190, 76-190, 77-190, 78-190, 79-190, 80-190, 81-190, 82-190, 83-190, 84-190, 85-190, 86-190, 87-190, 88-190, 89-190, 90-190, 91-190, 92-190, 93-190, 94-190, 95-190, 96-190, 97-190, 98-190, 99-190, 100-190, 101-190, 102-190, 103-190, 104-190, 105-190, 106-190, 107-190, 108-190, 109-190, 110-190, 111-190, 112-190, 113-190, 114-190, 115-190, 116-190, 117-190, 118-190, 119-190, 120-190, 121-190, 122-190, 123-190, 124-190, 125-190, 126-190, 127-190, 128-190, 129-190, 130-190, 131-190, 132-190, 133-190, 134-190, 135-190, 136-190, 137-190, 138-190, 139-190, 140-190, 141-190, 142-190, 143-190, 144-190, 145-190, 146-190, 147-190, 148-190, 149-190, 150-190, 151-190, 152-190, 153-190, 154-190, 155-190, 156-190, 157-190, 158-190, 159-190, 160-190, 161-190, 162-190, 163-190, 164-190, 165-190, 166-190, 167-190, 168-190, 169-190, 170-190, 171-190, 172-190, 173-190, 174-190, 175-190, 176-190, 177-190, 178-190, 179-190, 180-190, 181-190, 182-190, 183-190, 184-190, 185-190, 186-190, 21-189, 22-189, 23-189, 24-189, 25-189, 26-189, 27-189, 28-189, 29-189, 30-189, 31-189, 32-189, 33-189, 34-189, 35-189, 36-189, 37-189, 38-189, 39-189, 40-189, 41-189, 42-189, 43-189, 44-189, 45-189, 46-189, 47-189, 48-189, 49-189, 50-189, 51-189, 52-189, 53-189, 54-189, 55-189, 56-189, 57-189, 58-189, 59-189, 60-189, 61-189, 62-189, 63-189, 64-189, 65-189, 66-189, 67-189, 68-189, 69-189, 70-189, 71-189, 72-189, 73-189, 74-189, 75-189, 76-189, 77-189, 78-189, 79-189, 80-189, 81-189, 82-189, 83-189, 84-189, 85-189, 86-189, 87-189, 88-189, 89-189, 90-189, 91-189, 92-189, 93-189, 94-189, 95-189, 96-189, 97-189, 98-189, 99-189, 100-189, 101-189, 102-189, 103-189, 104-189, 105-189, 106-189, 107-189, 108-189, 109-189, 110-189, 111-189, 112-189, 113-189, 114-189, 115-189, 116-189, 117-189, 118-189, 119-189, 120-189, 121-189, 122-189, 123-189, 124-189, 125-189, 126-189, 127-189, 128-189, 129-189, 130-189, 131-189, 132-189, 133-189, 134-189, 135-189, 136-189, 137-189, 138-189, 139-189, 140-189, 141-189, 142-189, 143-189, 144-189, 145-189, 146-189, 147-189, 148-189, 149-189, 150-189, 151-189, 152-189, 153-189, 154-189, 155-189, 156-189, 157-189, 158-189, 159-189, 160-189, 161-189, 162-189, 163-189, 164-189, 165-189, 166-189, 167-189, 168-189, 169-189, 170-189, 171-189, 172-189, 173-189, 174-189, 175-189, 176-189, 177-189, 178-189, 179-189, 180-189, 181-189, 182-189, 183-189, 184-189, 185-189, 21-188, 22-188, 23-188, 24-188, 25-188, 26-188, 27-188, 28-188, 29-188, 30-188, 31-188, 32-188, 33-188, 34-188, 35-188, 36-188, 37-188, 38-188, 39-188, 40-188, 41-188, 42-188, 43-188, 44-188, 45-188, 46-188, 47-188, 48-188, 49-188, 50-188, 51-188, 52-188, 53-188, 54-188, 55-188, 56-188, 57-188, 58-188, 59-188, 60-188, 61-188, 62-188, 63-188, 64-188, 65-188, 66-188, 67-188, 68-188, 69-188, 70-188, 71-188, 72-188, 73-188, 74-188, 75-188, 76-188, 77-188, 78-188, 79-188, 80-188, 81-188, 82-188, 83-188, 84-188, 85-188, 86-188, 87-188, 88-188, 89-188, 90-188, 91-188, 92-188, 93-188, 94-188, 95-188, 96-188, 97-188, 98-188, 99-188, 100-188, 101-188, 102-188, 103-188, 104-188, 105-188, 106-188, 107-188, 108-188, 109-188, 110-188, 111-188, 112-188, 113-188, 114-188, 115-188, 116-188, 117-188, 118-188, 119-188, 120-188, 121-188, 122-188, 123-188, 124-188, 125-188, 126-188, 127-188, 128-188, 129-188, 130-188, 131-188, 132-188, 133-188, 134-188, 135-188, 136-188, 137-188, 138-188, 139-188, 140-188, 141-188, 142-188, 143-188, 144-188, 145-188, 146-188, 147-188, 148-188, 149-188, 150-188, 151-188, 152-188, 153-188, 154-188, 155-188, 156-188, 157-188, 158-188, 159-188, 160-188, 161-188, 162-188, 163-188, 164-188, 165-188, 166-188, 167-188, 168-188, 169-188, 170-188, 171-188, 172-188, 173-188, 174-188, 175-188, 176-188, 177-188, 178-188, 179-188, 180-188, 181-188, 182-188, 183-188, 184-188, 21-187, 22-187, 23-187, 24-187, 25-187, 26-187, 27-187, 28-187, 29-187, 30-187, 31-187, 32-187, 33-187, 34-187, 35-187, 36-187, 37-187, 38-187, 39-187, 40-187, 41-187, 42-187, 43-187, 44-187, 45-187, 46-187, 47-187, 48-187, 49-187, 50-187, 51-187, 52-187, 53-187, 54-187, 55-187, 56-187, 57-187, 58-187, 59-187, 60-187, 61-187, 62-187, 63-187, 64-187, 65-187, 66-187, 67-187, 68-187, 69-187, 70-187, 71-187, 72-187, 73-187, 74-187, 75-187, 76-187, 77-187, 78-187, 79-187, 80-187, 81-187, 82-187, 83-187, 84-187, 85-187, 86-187, 87-187, 88-187, 89-187, 90-187, 91-187, 92-187, 93-187, 94-187, 95-187, 96-187, 97-187, 98-187, 99-187, 100-187, 101-187, 102-187, 103-187, 104-187, 105-187, 106-187, 107-187, 108-187, 109-187, 110-187, 111-187, 112-187, 113-187, 114-187, 115-187, 116-187, 117-187, 118-187, 119-187, 120-187, 121-187, 122-187, 123-187, 124-187, 125-187, 126-187, 127-187, 128-187, 129-187, 130-187, 131-187, 132-187, 133-187, 134-187, 135-187, 136-187, 137-187, 138-187, 139-187, 140-187, 141-187, 142-187, 143-187, 144-187, 145-187, 146-187, 147-187, 148-187, 149-187, 150-187, 151-187, 152-187, 153-187, 154-187, 155-187, 156-187, 157-187, 158-187, 159-187, 160-187, 161-187, 162-187, 163-187, 164-187, 165-187, 166-187, 167-187, 168-187, 169-187, 170-187, 171-187, 172-187, 173-187, 174-187, 175-187, 176-187, 177-187, 178-187, 179-187, 180-187, 181-187, 182-187, 183-187, 21-186, 22-186, 23-186, 24-186, 25-186, 26-186, 27-186, 28-186, 29-186, 30-186, 31-186, 32-186, 33-186, 34-186, 35-186, 36-186, 37-186, 38-186, 39-186, 40-186, 41-186, 42-186, 43-186, 44-186, 45-186, 46-186, 47-186, 48-186, 49-186, 50-186, 51-186, 52-186, 53-186, 54-186, 55-186, 56-186, 57-186, 58-186, 59-186, 60-186, 61-186, 62-186, 63-186, 64-186, 65-186, 66-186, 67-186, 68-186, 69-186, 70-186, 71-186, 72-186, 73-186, 74-186, 75-186, 76-186, 77-186, 78-186, 79-186, 80-186, 81-186, 82-186, 83-186, 84-186, 85-186, 86-186, 87-186, 88-186, 89-186, 90-186, 91-186, 92-186, 93-186, 94-186, 95-186, 96-186, 97-186, 98-186, 99-186, 100-186, 101-186, 102-186, 103-186, 104-186, 105-186, 106-186, 107-186, 108-186, 109-186, 110-186, 111-186, 112-186, 113-186, 114-186, 115-186, 116-186, 117-186, 118-186, 119-186, 120-186, 121-186, 122-186, 123-186, 124-186, 125-186, 126-186, 127-186, 128-186, 129-186, 130-186, 131-186, 132-186, 133-186, 134-186, 135-186, 136-186, 137-186, 138-186, 139-186, 140-186, 141-186, 142-186, 143-186, 144-186, 145-186, 146-186, 147-186, 148-186, 149-186, 150-186, 151-186, 152-186, 153-186, 154-186, 155-186, 156-186, 157-186, 158-186, 159-186, 160-186, 161-186, 162-186, 163-186, 164-186, 165-186, 166-186, 167-186, 168-186, 169-186, 170-186, 171-186, 172-186, 173-186, 174-186, 175-186, 176-186, 177-186, 178-186, 179-186, 180-186, 181-186, 182-186, 21-185, 22-185, 23-185, 24-185, 25-185, 26-185, 27-185, 28-185, 29-185, 30-185, 31-185, 32-185, 33-185, 34-185, 35-185, 36-185, 37-185, 38-185, 39-185, 40-185, 41-185, 42-185, 43-185, 44-185, 45-185, 46-185, 47-185, 48-185, 49-185, 50-185, 51-185, 52-185, 53-185, 54-185, 55-185, 56-185, 57-185, 58-185, 59-185, 60-185, 61-185, 62-185, 63-185, 64-185, 65-185, 66-185, 67-185, 68-185, 69-185, 70-185, 71-185, 72-185, 73-185, 74-185, 75-185, 76-185, 77-185, 78-185, 79-185, 80-185, 81-185, 82-185, 83-185, 84-185, 85-185, 86-185, 87-185, 88-185, 89-185, 90-185, 91-185, 92-185, 93-185, 94-185, 95-185, 96-185, 97-185, 98-185, 99-185, 100-185, 101-185, 102-185, 103-185, 104-185, 105-185, 106-185, 107-185, 108-185, 109-185, 110-185, 111-185, 112-185, 113-185, 114-185, 115-185, 116-185, 117-185, 118-185, 119-185, 120-185, 121-185, 122-185, 123-185, 124-185, 125-185, 126-185, 127-185, 128-185, 129-185, 130-185, 131-185, 132-185, 133-185, 134-185, 135-185, 136-185, 137-185, 138-185, 139-185, 140-185, 141-185, 142-185, 143-185, 144-185, 145-185, 146-185, 147-185, 148-185, 149-185, 150-185, 151-185, 152-185, 153-185, 154-185, 155-185, 156-185, 157-185, 158-185, 159-185, 160-185, 161-185, 162-185, 163-185, 164-185, 165-185, 166-185, 167-185, 168-185, 169-185, 170-185, 171-185, 172-185, 173-185, 174-185, 175-185, 176-185, 177-185, 178-185, 179-185, 180-185, 181-185, 21-184, 22-184, 23-184, 24-184, 25-184, 26-184, 27-184, 28-184, 29-184, 30-184, 31-184, 32-184, 33-184, 34-184, 35-184, 36-184, 37-184, 38-184, 39-184, 40-184, 41-184, 42-184, 43-184, 44-184, 45-184, 46-184, 47-184, 48-184, 49-184, 50-184, 51-184, 52-184, 53-184, 54-184, 55-184, 56-184, 57-184, 58-184, 59-184, 60-184, 61-184, 62-184, 63-184, 64-184, 65-184, 66-184, 67-184, 68-184, 69-184, 70-184, 71-184, 72-184, 73-184, 74-184, 75-184, 76-184, 77-184, 78-184, 79-184, 80-184, 81-184, 82-184, 83-184, 84-184, 85-184, 86-184, 87-184, 88-184, 89-184, 90-184, 91-184, 92-184, 93-184, 94-184, 95-184, 96-184, 97-184, 98-184, 99-184, 100-184, 101-184, 102-184, 103-184, 104-184, 105-184, 106-184, 107-184, 108-184, 109-184, 110-184, 111-184, 112-184, 113-184, 114-184, 115-184, 116-184, 117-184, 118-184, 119-184, 120-184, 121-184, 122-184, 123-184, 124-184, 125-184, 126-184, 127-184, 128-184, 129-184, 130-184, 131-184, 132-184, 133-184, 134-184, 135-184, 136-184, 137-184, 138-184, 139-184, 140-184, 141-184, 142-184, 143-184, 144-184, 145-184, 146-184, 147-184, 148-184, 149-184, 150-184, 151-184, 152-184, 153-184, 154-184, 155-184, 156-184, 157-184, 158-184, 159-184, 160-184, 161-184, 162-184, 163-184, 164-184, 165-184, 166-184, 167-184, 168-184, 169-184, 170-184, 171-184, 172-184, 173-184, 174-184, 175-184, 176-184, 177-184, 178-184, 179-184, 180-184, 21-183, 22-183, 23-183, 24-183, 25-183, 26-183, 27-183, 28-183, 29-183, 30-183, 31-183, 32-183, 33-183, 34-183, 35-183, 36-183, 37-183, 38-183, 39-183, 40-183, 41-183, 42-183, 43-183, 44-183, 45-183, 46-183, 47-183, 48-183, 49-183, 50-183, 51-183, 52-183, 53-183, 54-183, 55-183, 56-183, 57-183, 58-183, 59-183, 60-183, 61-183, 62-183, 63-183, 64-183, 65-183, 66-183, 67-183, 68-183, 69-183, 70-183, 71-183, 72-183, 73-183, 74-183, 75-183, 76-183, 77-183, 78-183, 79-183, 80-183, 81-183, 82-183, 83-183, 84-183, 85-183, 86-183, 87-183, 88-183, 89-183, 90-183, 91-183, 92-183, 93-183, 94-183, 95-183, 96-183, 97-183, 98-183, 99-183, 100-183, 101-183, 102-183, 103-183, 104-183, 105-183, 106-183, 107-183, 108-183, 109-183, 110-183, 111-183, 112-183, 113-183, 114-183, 115-183, 116-183, 117-183, 118-183, 119-183, 120-183, 121-183, 122-183, 123-183, 124-183, 125-183, 126-183, 127-183, 128-183, 129-183, 130-183, 131-183, 132-183, 133-183, 134-183, 135-183, 136-183, 137-183, 138-183, 139-183, 140-183, 141-183, 142-183, 143-183, 144-183, 145-183, 146-183, 147-183, 148-183, 149-183, 150-183, 151-183, 152-183, 153-183, 154-183, 155-183, 156-183, 157-183, 158-183, 159-183, 160-183, 161-183, 162-183, 163-183, 164-183, 165-183, 166-183, 167-183, 168-183, 169-183, 170-183, 171-183, 172-183, 173-183, 174-183, 175-183, 176-183, 177-183, 178-183, 179-183, 21-182, 22-182, 23-182, 24-182, 25-182, 26-182, 27-182, 28-182, 29-182, 30-182, 31-182, 32-182, 33-182, 34-182, 35-182, 36-182, 37-182, 38-182, 39-182, 40-182, 41-182, 42-182, 43-182, 44-182, 45-182, 46-182, 47-182, 48-182, 49-182, 50-182, 51-182, 52-182, 53-182, 54-182, 55-182, 56-182, 57-182, 58-182, 59-182, 60-182, 61-182, 62-182, 63-182, 64-182, 65-182, 66-182, 67-182, 68-182, 69-182, 70-182, 71-182, 72-182, 73-182, 74-182, 75-182, 76-182, 77-182, 78-182, 79-182, 80-182, 81-182, 82-182, 83-182, 84-182, 85-182, 86-182, 87-182, 88-182, 89-182, 90-182, 91-182, 92-182, 93-182, 94-182, 95-182, 96-182, 97-182, 98-182, 99-182, 100-182, 101-182, 102-182, 103-182, 104-182, 105-182, 106-182, 107-182, 108-182, 109-182, 110-182, 111-182, 112-182, 113-182, 114-182, 115-182, 116-182, 117-182, 118-182, 119-182, 120-182, 121-182, 122-182, 123-182, 124-182, 125-182, 126-182, 127-182, 128-182, 129-182, 130-182, 131-182, 132-182, 133-182, 134-182, 135-182, 136-182, 137-182, 138-182, 139-182, 140-182, 141-182, 142-182, 143-182, 144-182, 145-182, 146-182, 147-182, 148-182, 149-182, 150-182, 151-182, 152-182, 153-182, 154-182, 155-182, 156-182, 157-182, 158-182, 159-182, 160-182, 161-182, 162-182, 163-182, 164-182, 165-182, 166-182, 167-182, 168-182, 169-182, 170-182, 171-182, 172-182, 173-182, 174-182, 175-182, 176-182, 177-182, 178-182, 21-181, 22-181, 23-181, 24-181, 25-181, 26-181, 27-181, 28-181, 29-181, 30-181, 31-181, 32-181, 33-181, 34-181, 35-181, 36-181, 37-181, 38-181, 39-181, 40-181, 41-181, 42-181, 43-181, 44-181, 45-181, 46-181, 47-181, 48-181, 49-181, 50-181, 51-181, 52-181, 53-181, 54-181, 55-181, 56-181, 57-181, 58-181, 59-181, 60-181, 61-181, 62-181, 63-181, 64-181, 65-181, 66-181, 67-181, 68-181, 69-181, 70-181, 71-181, 72-181, 73-181, 74-181, 75-181, 76-181, 77-181, 78-181, 79-181, 80-181, 81-181, 82-181, 83-181, 84-181, 85-181, 86-181, 87-181, 88-181, 89-181, 90-181, 91-181, 92-181, 93-181, 94-181, 95-181, 96-181, 97-181, 98-181, 99-181, 100-181, 101-181, 102-181, 103-181, 104-181, 105-181, 106-181, 107-181, 108-181, 109-181, 110-181, 111-181, 112-181, 113-181, 114-181, 115-181, 116-181, 117-181, 118-181, 119-181, 120-181, 121-181, 122-181, 123-181, 124-181, 125-181, 126-181, 127-181, 128-181, 129-181, 130-181, 131-181, 132-181, 133-181, 134-181, 135-181, 136-181, 137-181, 138-181, 139-181, 140-181, 141-181, 142-181, 143-181, 144-181, 145-181, 146-181, 147-181, 148-181, 149-181, 150-181, 151-181, 152-181, 153-181, 154-181, 155-181, 156-181, 157-181, 158-181, 159-181, 160-181, 161-181, 162-181, 163-181, 164-181, 165-181, 166-181, 167-181, 168-181, 169-181, 170-181, 171-181, 172-181, 173-181, 174-181, 175-181, 176-181, 177-181, 21-180, 22-180, 23-180, 24-180, 25-180, 26-180, 27-180, 28-180, 29-180, 30-180, 31-180, 32-180, 33-180, 34-180, 35-180, 36-180, 37-180, 38-180, 39-180, 40-180, 41-180, 42-180, 43-180, 44-180, 45-180, 46-180, 47-180, 48-180, 49-180, 50-180, 51-180, 52-180, 53-180, 54-180, 55-180, 56-180, 57-180, 58-180, 59-180, 60-180, 61-180, 62-180, 63-180, 64-180, 65-180, 66-180, 67-180, 68-180, 69-180, 70-180, 71-180, 72-180, 73-180, 74-180, 75-180, 76-180, 77-180, 78-180, 79-180, 80-180, 81-180, 82-180, 83-180, 84-180, 85-180, 86-180, 87-180, 88-180, 89-180, 90-180, 91-180, 92-180, 93-180, 94-180, 95-180, 96-180, 97-180, 98-180, 99-180, 100-180, 101-180, 102-180, 103-180, 104-180, 105-180, 106-180, 107-180, 108-180, 109-180, 110-180, 111-180, 112-180, 113-180, 114-180, 115-180, 116-180, 117-180, 118-180, 119-180, 120-180, 121-180, 122-180, 123-180, 124-180, 125-180, 126-180, 127-180, 128-180, 129-180, 130-180, 131-180, 132-180, 133-180, 134-180, 135-180, 136-180, 137-180, 138-180, 139-180, 140-180, 141-180, 142-180, 143-180, 144-180, 145-180, 146-180, 147-180, 148-180, 149-180, 150-180, 151-180, 152-180, 153-180, 154-180, 155-180, 156-180, 157-180, 158-180, 159-180, 160-180, 161-180, 162-180, 163-180, 164-180, 165-180, 166-180, 167-180, 168-180, 169-180, 170-180, 171-180, 172-180, 173-180, 174-180, 175-180, 176-180, 21-179, 22-179, 23-179, 24-179, 25-179, 26-179, 27-179, 28-179, 29-179, 30-179, 31-179, 32-179, 33-179, 34-179, 35-179, 36-179, 37-179, 38-179, 39-179, 40-179, 41-179, 42-179, 43-179, 44-179, 45-179, 46-179, 47-179, 48-179, 49-179, 50-179, 51-179, 52-179, 53-179, 54-179, 55-179, 56-179, 57-179, 58-179, 59-179, 60-179, 61-179, 62-179, 63-179, 64-179, 65-179, 66-179, 67-179, 68-179, 69-179, 70-179, 71-179, 72-179, 73-179, 74-179, 75-179, 76-179, 77-179, 78-179, 79-179, 80-179, 81-179, 82-179, 83-179, 84-179, 85-179, 86-179, 87-179, 88-179, 89-179, 90-179, 91-179, 92-179, 93-179, 94-179, 95-179, 96-179, 97-179, 98-179, 99-179, 100-179, 101-179, 102-179, 103-179, 104-179, 105-179, 106-179, 107-179, 108-179, 109-179, 110-179, 111-179, 112-179, 113-179, 114-179, 115-179, 116-179, 117-179, 118-179, 119-179, 120-179, 121-179, 122-179, 123-179, 124-179, 125-179, 126-179, 127-179, 128-179, 129-179, 130-179, 131-179, 132-179, 133-179, 134-179, 135-179, 136-179, 137-179, 138-179, 139-179, 140-179, 141-179, 142-179, 143-179, 144-179, 145-179, 146-179, 147-179, 148-179, 149-179, 150-179, 151-179, 152-179, 153-179, 154-179, 155-179, 156-179, 157-179, 158-179, 159-179, 160-179, 161-179, 162-179, 163-179, 164-179, 165-179, 166-179, 167-179, 168-179, 169-179, 170-179, 171-179, 172-179, 173-179, 174-179, 175-179, 21-178, 22-178, 23-178, 24-178, 25-178, 26-178, 27-178, 28-178, 29-178, 30-178, 31-178, 32-178, 33-178, 34-178, 35-178, 36-178, 37-178, 38-178, 39-178, 40-178, 41-178, 42-178, 43-178, 44-178, 45-178, 46-178, 47-178, 48-178, 49-178, 50-178, 51-178, 52-178, 53-178, 54-178, 55-178, 56-178, 57-178, 58-178, 59-178, 60-178, 61-178, 62-178, 63-178, 64-178, 65-178, 66-178, 67-178, 68-178, 69-178, 70-178, 71-178, 72-178, 73-178, 74-178, 75-178, 76-178, 77-178, 78-178, 79-178, 80-178, 81-178, 82-178, 83-178, 84-178, 85-178, 86-178, 87-178, 88-178, 89-178, 90-178, 91-178, 92-178, 93-178, 94-178, 95-178, 96-178, 97-178, 98-178, 99-178, 100-178, 101-178, 102-178, 103-178, 104-178, 105-178, 106-178, 107-178, 108-178, 109-178, 110-178, 111-178, 112-178, 113-178, 114-178, 115-178, 116-178, 117-178, 118-178, 119-178, 120-178, 121-178, 122-178, 123-178, 124-178, 125-178, 126-178, 127-178, 128-178, 129-178, 130-178, 131-178, 132-178, 133-178, 134-178, 135-178, 136-178, 137-178, 138-178, 139-178, 140-178, 141-178, 142-178, 143-178, 144-178, 145-178, 146-178, 147-178, 148-178, 149-178, 150-178, 151-178, 152-178, 153-178, 154-178, 155-178, 156-178, 157-178, 158-178, 159-178, 160-178, 161-178, 162-178, 163-178, 164-178, 165-178, 166-178, 167-178, 168-178, 169-178, 170-178, 171-178, 172-178, 173-178, 174-178, 21-177, 22-177, 23-177, 24-177, 25-177, 26-177, 27-177, 28-177, 29-177, 30-177, 31-177, 32-177, 33-177, 34-177, 35-177, 36-177, 37-177, 38-177, 39-177, 40-177, 41-177, 42-177, 43-177, 44-177, 45-177, 46-177, 47-177, 48-177, 49-177, 50-177, 51-177, 52-177, 53-177, 54-177, 55-177, 56-177, 57-177, 58-177, 59-177, 60-177, 61-177, 62-177, 63-177, 64-177, 65-177, 66-177, 67-177, 68-177, 69-177, 70-177, 71-177, 72-177, 73-177, 74-177, 75-177, 76-177, 77-177, 78-177, 79-177, 80-177, 81-177, 82-177, 83-177, 84-177, 85-177, 86-177, 87-177, 88-177, 89-177, 90-177, 91-177, 92-177, 93-177, 94-177, 95-177, 96-177, 97-177, 98-177, 99-177, 100-177, 101-177, 102-177, 103-177, 104-177, 105-177, 106-177, 107-177, 108-177, 109-177, 110-177, 111-177, 112-177, 113-177, 114-177, 115-177, 116-177, 117-177, 118-177, 119-177, 120-177, 121-177, 122-177, 123-177, 124-177, 125-177, 126-177, 127-177, 128-177, 129-177, 130-177, 131-177, 132-177, 133-177, 134-177, 135-177, 136-177, 137-177, 138-177, 139-177, 140-177, 141-177, 142-177, 143-177, 144-177, 145-177, 146-177, 147-177, 148-177, 149-177, 150-177, 151-177, 152-177, 153-177, 154-177, 155-177, 156-177, 157-177, 158-177, 159-177, 160-177, 161-177, 162-177, 163-177, 164-177, 165-177, 166-177, 167-177, 168-177, 169-177, 170-177, 171-177, 172-177, 173-177, 21-176, 22-176, 23-176, 24-176, 25-176, 26-176, 27-176, 28-176, 29-176, 30-176, 31-176, 32-176, 33-176, 34-176, 35-176, 36-176, 37-176, 38-176, 39-176, 40-176, 41-176, 42-176, 43-176, 44-176, 45-176, 46-176, 47-176, 48-176, 49-176, 50-176, 51-176, 52-176, 53-176, 54-176, 55-176, 56-176, 57-176, 58-176, 59-176, 60-176, 61-176, 62-176, 63-176, 64-176, 65-176, 66-176, 67-176, 68-176, 69-176, 70-176, 71-176, 72-176, 73-176, 74-176, 75-176, 76-176, 77-176, 78-176, 79-176, 80-176, 81-176, 82-176, 83-176, 84-176, 85-176, 86-176, 87-176, 88-176, 89-176, 90-176, 91-176, 92-176, 93-176, 94-176, 95-176, 96-176, 97-176, 98-176, 99-176, 100-176, 101-176, 102-176, 103-176, 104-176, 105-176, 106-176, 107-176, 108-176, 109-176, 110-176, 111-176, 112-176, 113-176, 114-176, 115-176, 116-176, 117-176, 118-176, 119-176, 120-176, 121-176, 122-176, 123-176, 124-176, 125-176, 126-176, 127-176, 128-176, 129-176, 130-176, 131-176, 132-176, 133-176, 134-176, 135-176, 136-176, 137-176, 138-176, 139-176, 140-176, 141-176, 142-176, 143-176, 144-176, 145-176, 146-176, 147-176, 148-176, 149-176, 150-176, 151-176, 152-176, 153-176, 154-176, 155-176, 156-176, 157-176, 158-176, 159-176, 160-176, 161-176, 162-176, 163-176, 164-176, 165-176, 166-176, 167-176, 168-176, 169-176, 170-176, 171-176, 172-176, 21-175, 22-175, 23-175, 24-175, 25-175, 26-175, 27-175, 28-175, 29-175, 30-175, 31-175, 32-175, 33-175, 34-175, 35-175, 36-175, 37-175, 38-175, 39-175, 40-175, 41-175, 42-175, 43-175, 44-175, 45-175, 46-175, 47-175, 48-175, 49-175, 50-175, 51-175, 52-175, 53-175, 54-175, 55-175, 56-175, 57-175, 58-175, 59-175, 60-175, 61-175, 62-175, 63-175, 64-175, 65-175, 66-175, 67-175, 68-175, 69-175, 70-175, 71-175, 72-175, 73-175, 74-175, 75-175, 76-175, 77-175, 78-175, 79-175, 80-175, 81-175, 82-175, 83-175, 84-175, 85-175, 86-175, 87-175, 88-175, 89-175, 90-175, 91-175, 92-175, 93-175, 94-175, 95-175, 96-175, 97-175, 98-175, 99-175, 100-175, 101-175, 102-175, 103-175, 104-175, 105-175, 106-175, 107-175, 108-175, 109-175, 110-175, 111-175, 112-175, 113-175, 114-175, 115-175, 116-175, 117-175, 118-175, 119-175, 120-175, 121-175, 122-175, 123-175, 124-175, 125-175, 126-175, 127-175, 128-175, 129-175, 130-175, 131-175, 132-175, 133-175, 134-175, 135-175, 136-175, 137-175, 138-175, 139-175, 140-175, 141-175, 142-175, 143-175, 144-175, 145-175, 146-175, 147-175, 148-175, 149-175, 150-175, 151-175, 152-175, 153-175, 154-175, 155-175, 156-175, 157-175, 158-175, 159-175, 160-175, 161-175, 162-175, 163-175, 164-175, 165-175, 166-175, 167-175, 168-175, 169-175, 170-175, 171-175, 21-174, 22-174, 23-174, 24-174, 25-174, 26-174, 27-174, 28-174, 29-174, 30-174, 31-174, 32-174, 33-174, 34-174, 35-174, 36-174, 37-174, 38-174, 39-174, 40-174, 41-174, 42-174, 43-174, 44-174, 45-174, 46-174, 47-174, 48-174, 49-174, 50-174, 51-174, 52-174, 53-174, 54-174, 55-174, 56-174, 57-174, 58-174, 59-174, 60-174, 61-174, 62-174, 63-174, 64-174, 65-174, 66-174, 67-174, 68-174, 69-174, 70-174, 71-174, 72-174, 73-174, 74-174, 75-174, 76-174, 77-174, 78-174, 79-174, 80-174, 81-174, 82-174, 83-174, 84-174, 85-174, 86-174, 87-174, 88-174, 89-174, 90-174, 91-174, 92-174, 93-174, 94-174, 95-174, 96-174, 97-174, 98-174, 99-174, 100-174, 101-174, 102-174, 103-174, 104-174, 105-174, 106-174, 107-174, 108-174, 109-174, 110-174, 111-174, 112-174, 113-174, 114-174, 115-174, 116-174, 117-174, 118-174, 119-174, 120-174, 121-174, 122-174, 123-174, 124-174, 125-174, 126-174, 127-174, 128-174, 129-174, 130-174, 131-174, 132-174, 133-174, 134-174, 135-174, 136-174, 137-174, 138-174, 139-174, 140-174, 141-174, 142-174, 143-174, 144-174, 145-174, 146-174, 147-174, 148-174, 149-174, 150-174, 151-174, 152-174, 153-174, 154-174, 155-174, 156-174, 157-174, 158-174, 159-174, 160-174, 161-174, 162-174, 163-174, 164-174, 165-174, 166-174, 167-174, 168-174, 169-174, 170-174, 21-173, 22-173, 23-173, 24-173, 25-173, 26-173, 27-173, 28-173, 29-173, 30-173, 31-173, 32-173, 33-173, 34-173, 35-173, 36-173, 37-173, 38-173, 39-173, 40-173, 41-173, 42-173, 43-173, 44-173, 45-173, 46-173, 47-173, 48-173, 49-173, 50-173, 51-173, 52-173, 53-173, 54-173, 55-173, 56-173, 57-173, 58-173, 59-173, 60-173, 61-173, 62-173, 63-173, 64-173, 65-173, 66-173, 67-173, 68-173, 69-173, 70-173, 71-173, 72-173, 73-173, 74-173, 75-173, 76-173, 77-173, 78-173, 79-173, 80-173, 81-173, 82-173, 83-173, 84-173, 85-173, 86-173, 87-173, 88-173, 89-173, 90-173, 91-173, 92-173, 93-173, 94-173, 95-173, 96-173, 97-173, 98-173, 99-173, 100-173, 101-173, 102-173, 103-173, 104-173, 105-173, 106-173, 107-173, 108-173, 109-173, 110-173, 111-173, 112-173, 113-173, 114-173, 115-173, 116-173, 117-173, 118-173, 119-173, 120-173, 121-173, 122-173, 123-173, 124-173, 125-173, 126-173, 127-173, 128-173, 129-173, 130-173, 131-173, 132-173, 133-173, 134-173, 135-173, 136-173, 137-173, 138-173, 139-173, 140-173, 141-173, 142-173, 143-173, 144-173, 145-173, 146-173, 147-173, 148-173, 149-173, 150-173, 151-173, 152-173, 153-173, 154-173, 155-173, 156-173, 157-173, 158-173, 159-173, 160-173, 161-173, 162-173, 163-173, 164-173, 165-173, 166-173, 167-173, 168-173, 169-173, 21-172, 22-172, 23-172, 24-172, 25-172, 26-172, 27-172, 28-172, 29-172, 30-172, 31-172, 32-172, 33-172, 34-172, 35-172, 36-172, 37-172, 38-172, 39-172, 40-172, 41-172, 42-172, 43-172, 44-172, 45-172, 46-172, 47-172, 48-172, 49-172, 50-172, 51-172, 52-172, 53-172, 54-172, 55-172, 56-172, 57-172, 58-172, 59-172, 60-172, 61-172, 62-172, 63-172, 64-172, 65-172, 66-172, 67-172, 68-172, 69-172, 70-172, 71-172, 72-172, 73-172, 74-172, 75-172, 76-172, 77-172, 78-172, 79-172, 80-172, 81-172, 82-172, 83-172, 84-172, 85-172, 86-172, 87-172, 88-172, 89-172, 90-172, 91-172, 92-172, 93-172, 94-172, 95-172, 96-172, 97-172, 98-172, 99-172, 100-172, 101-172, 102-172, 103-172, 104-172, 105-172, 106-172, 107-172, 108-172, 109-172, 110-172, 111-172, 112-172, 113-172, 114-172, 115-172, 116-172, 117-172, 118-172, 119-172, 120-172, 121-172, 122-172, 123-172, 124-172, 125-172, 126-172, 127-172, 128-172, 129-172, 130-172, 131-172, 132-172, 133-172, 134-172, 135-172, 136-172, 137-172, 138-172, 139-172, 140-172, 141-172, 142-172, 143-172, 144-172, 145-172, 146-172, 147-172, 148-172, 149-172, 150-172, 151-172, 152-172, 153-172, 154-172, 155-172, 156-172, 157-172, 158-172, 159-172, 160-172, 161-172, 162-172, 163-172, 164-172, 165-172, 166-172, 167-172, 168-172, 21-171, 22-171, 23-171, 24-171, 25-171, 26-171, 27-171, 28-171, 29-171, 30-171, 31-171, 32-171, 33-171, 34-171, 35-171, 36-171, 37-171, 38-171, 39-171, 40-171, 41-171, 42-171, 43-171, 44-171, 45-171, 46-171, 47-171, 48-171, 49-171, 50-171, 51-171, 52-171, 53-171, 54-171, 55-171, 56-171, 57-171, 58-171, 59-171, 60-171, 61-171, 62-171, 63-171, 64-171, 65-171, 66-171, 67-171, 68-171, 69-171, 70-171, 71-171, 72-171, 73-171, 74-171, 75-171, 76-171, 77-171, 78-171, 79-171, 80-171, 81-171, 82-171, 83-171, 84-171, 85-171, 86-171, 87-171, 88-171, 89-171, 90-171, 91-171, 92-171, 93-171, 94-171, 95-171, 96-171, 97-171, 98-171, 99-171, 100-171, 101-171, 102-171, 103-171, 104-171, 105-171, 106-171, 107-171, 108-171, 109-171, 110-171, 111-171, 112-171, 113-171, 114-171, 115-171, 116-171, 117-171, 118-171, 119-171, 120-171, 121-171, 122-171, 123-171, 124-171, 125-171, 126-171, 127-171, 128-171, 129-171, 130-171, 131-171, 132-171, 133-171, 134-171, 135-171, 136-171, 137-171, 138-171, 139-171, 140-171, 141-171, 142-171, 143-171, 144-171, 145-171, 146-171, 147-171, 148-171, 149-171, 150-171, 151-171, 152-171, 153-171, 154-171, 155-171, 156-171, 157-171, 158-171, 159-171, 160-171, 161-171, 162-171, 163-171, 164-171, 165-171, 166-171, 167-171, 21-170, 22-170, 23-170, 24-170, 25-170, 26-170, 27-170, 28-170, 29-170, 30-170, 31-170, 32-170, 33-170, 34-170, 35-170, 36-170, 37-170, 38-170, 39-170, 40-170, 41-170, 42-170, 43-170, 44-170, 45-170, 46-170, 47-170, 48-170, 49-170, 50-170, 51-170, 52-170, 53-170, 54-170, 55-170, 56-170, 57-170, 58-170, 59-170, 60-170, 61-170, 62-170, 63-170, 64-170, 65-170, 66-170, 67-170, 68-170, 69-170, 70-170, 71-170, 72-170, 73-170, 74-170, 75-170, 76-170, 77-170, 78-170, 79-170, 80-170, 81-170, 82-170, 83-170, 84-170, 85-170, 86-170, 87-170, 88-170, 89-170, 90-170, 91-170, 92-170, 93-170, 94-170, 95-170, 96-170, 97-170, 98-170, 99-170, 100-170, 101-170, 102-170, 103-170, 104-170, 105-170, 106-170, 107-170, 108-170, 109-170, 110-170, 111-170, 112-170, 113-170, 114-170, 115-170, 116-170, 117-170, 118-170, 119-170, 120-170, 121-170, 122-170, 123-170, 124-170, 125-170, 126-170, 127-170, 128-170, 129-170, 130-170, 131-170, 132-170, 133-170, 134-170, 135-170, 136-170, 137-170, 138-170, 139-170, 140-170, 141-170, 142-170, 143-170, 144-170, 145-170, 146-170, 147-170, 148-170, 149-170, 150-170, 151-170, 152-170, 153-170, 154-170, 155-170, 156-170, 157-170, 158-170, 159-170, 160-170, 161-170, 162-170, 163-170, 164-170, 165-170, 166-170, 21-169, 22-169, 23-169, 24-169, 25-169, 26-169, 27-169, 28-169, 29-169, 30-169, 31-169, 32-169, 33-169, 34-169, 35-169, 36-169, 37-169, 38-169, 39-169, 40-169, 41-169, 42-169, 43-169, 44-169, 45-169, 46-169, 47-169, 48-169, 49-169, 50-169, 51-169, 52-169, 53-169, 54-169, 55-169, 56-169, 57-169, 58-169, 59-169, 60-169, 61-169, 62-169, 63-169, 64-169, 65-169, 66-169, 67-169, 68-169, 69-169, 70-169, 71-169, 72-169, 73-169, 74-169, 75-169, 76-169, 77-169, 78-169, 79-169, 80-169, 81-169, 82-169, 83-169, 84-169, 85-169, 86-169, 87-169, 88-169, 89-169, 90-169, 91-169, 92-169, 93-169, 94-169, 95-169, 96-169, 97-169, 98-169, 99-169, 100-169, 101-169, 102-169, 103-169, 104-169, 105-169, 106-169, 107-169, 108-169, 109-169, 110-169, 111-169, 112-169, 113-169, 114-169, 115-169, 116-169, 117-169, 118-169, 119-169, 120-169, 121-169, 122-169, 123-169, 124-169, 125-169, 126-169, 127-169, 128-169, 129-169, 130-169, 131-169, 132-169, 133-169, 134-169, 135-169, 136-169, 137-169, 138-169, 139-169, 140-169, 141-169, 142-169, 143-169, 144-169, 145-169, 146-169, 147-169, 148-169, 149-169, 150-169, 151-169, 152-169, 153-169, 154-169, 155-169, 156-169, 157-169, 158-169, 159-169, 160-169, 161-169, 162-169, 163-169, 164-169, 165-169, 21-168, 22-168, 23-168, 24-168, 25-168, 26-168, 27-168, 28-168, 29-168, 30-168, 31-168, 32-168, 33-168, 34-168, 35-168, 36-168, 37-168, 38-168, 39-168, 40-168, 41-168, 42-168, 43-168, 44-168, 45-168, 46-168, 47-168, 48-168, 49-168, 50-168, 51-168, 52-168, 53-168, 54-168, 55-168, 56-168, 57-168, 58-168, 59-168, 60-168, 61-168, 62-168, 63-168, 64-168, 65-168, 66-168, 67-168, 68-168, 69-168, 70-168, 71-168, 72-168, 73-168, 74-168, 75-168, 76-168, 77-168, 78-168, 79-168, 80-168, 81-168, 82-168, 83-168, 84-168, 85-168, 86-168, 87-168, 88-168, 89-168, 90-168, 91-168, 92-168, 93-168, 94-168, 95-168, 96-168, 97-168, 98-168, 99-168, 100-168, 101-168, 102-168, 103-168, 104-168, 105-168, 106-168, 107-168, 108-168, 109-168, 110-168, 111-168, 112-168, 113-168, 114-168, 115-168, 116-168, 117-168, 118-168, 119-168, 120-168, 121-168, 122-168, 123-168, 124-168, 125-168, 126-168, 127-168, 128-168, 129-168, 130-168, 131-168, 132-168, 133-168, 134-168, 135-168, 136-168, 137-168, 138-168, 139-168, 140-168, 141-168, 142-168, 143-168, 144-168, 145-168, 146-168, 147-168, 148-168, 149-168, 150-168, 151-168, 152-168, 153-168, 154-168, 155-168, 156-168, 157-168, 158-168, 159-168, 160-168, 161-168, 162-168, 163-168, 164-168, 21-167, 22-167, 23-167, 24-167, 25-167, 26-167, 27-167, 28-167, 29-167, 30-167, 31-167, 32-167, 33-167, 34-167, 35-167, 36-167, 37-167, 38-167, 39-167, 40-167, 41-167, 42-167, 43-167, 44-167, 45-167, 46-167, 47-167, 48-167, 49-167, 50-167, 51-167, 52-167, 53-167, 54-167, 55-167, 56-167, 57-167, 58-167, 59-167, 60-167, 61-167, 62-167, 63-167, 64-167, 65-167, 66-167, 67-167, 68-167, 69-167, 70-167, 71-167, 72-167, 73-167, 74-167, 75-167, 76-167, 77-167, 78-167, 79-167, 80-167, 81-167, 82-167, 83-167, 84-167, 85-167, 86-167, 87-167, 88-167, 89-167, 90-167, 91-167, 92-167, 93-167, 94-167, 95-167, 96-167, 97-167, 98-167, 99-167, 100-167, 101-167, 102-167, 103-167, 104-167, 105-167, 106-167, 107-167, 108-167, 109-167, 110-167, 111-167, 112-167, 113-167, 114-167, 115-167, 116-167, 117-167, 118-167, 119-167, 120-167, 121-167, 122-167, 123-167, 124-167, 125-167, 126-167, 127-167, 128-167, 129-167, 130-167, 131-167, 132-167, 133-167, 134-167, 135-167, 136-167, 137-167, 138-167, 139-167, 140-167, 141-167, 142-167, 143-167, 144-167, 145-167, 146-167, 147-167, 148-167, 149-167, 150-167, 151-167, 152-167, 153-167, 154-167, 155-167, 156-167, 157-167, 158-167, 159-167, 160-167, 161-167, 162-167, 163-167, 21-166, 22-166, 23-166, 24-166, 25-166, 26-166, 27-166, 28-166, 29-166, 30-166, 31-166, 32-166, 33-166, 34-166, 35-166, 36-166, 37-166, 38-166, 39-166, 40-166, 41-166, 42-166, 43-166, 44-166, 45-166, 46-166, 47-166, 48-166, 49-166, 50-166, 51-166, 52-166, 53-166, 54-166, 55-166, 56-166, 57-166, 58-166, 59-166, 60-166, 61-166, 62-166, 63-166, 64-166, 65-166, 66-166, 67-166, 68-166, 69-166, 70-166, 71-166, 72-166, 73-166, 74-166, 75-166, 76-166, 77-166, 78-166, 79-166, 80-166, 81-166, 82-166, 83-166, 84-166, 85-166, 86-166, 87-166, 88-166, 89-166, 90-166, 91-166, 92-166, 93-166, 94-166, 95-166, 96-166, 97-166, 98-166, 99-166, 100-166, 101-166, 102-166, 103-166, 104-166, 105-166, 106-166, 107-166, 108-166, 109-166, 110-166, 111-166, 112-166, 113-166, 114-166, 115-166, 116-166, 117-166, 118-166, 119-166, 120-166, 121-166, 122-166, 123-166, 124-166, 125-166, 126-166, 127-166, 128-166, 129-166, 130-166, 131-166, 132-166, 133-166, 134-166, 135-166, 136-166, 137-166, 138-166, 139-166, 140-166, 141-166, 142-166, 143-166, 144-166, 145-166, 146-166, 147-166, 148-166, 149-166, 150-166, 151-166, 152-166, 153-166, 154-166, 155-166, 156-166, 157-166, 158-166, 159-166, 160-166, 161-166, 162-166, 21-165, 22-165, 23-165, 24-165, 25-165, 26-165, 27-165, 28-165, 29-165, 30-165, 31-165, 32-165, 33-165, 34-165, 35-165, 36-165, 37-165, 38-165, 39-165, 40-165, 41-165, 42-165, 43-165, 44-165, 45-165, 46-165, 47-165, 48-165, 49-165, 50-165, 51-165, 52-165, 53-165, 54-165, 55-165, 56-165, 57-165, 58-165, 59-165, 60-165, 61-165, 62-165, 63-165, 64-165, 65-165, 66-165, 67-165, 68-165, 69-165, 70-165, 71-165, 72-165, 73-165, 74-165, 75-165, 76-165, 77-165, 78-165, 79-165, 80-165, 81-165, 82-165, 83-165, 84-165, 85-165, 86-165, 87-165, 88-165, 89-165, 90-165, 91-165, 92-165, 93-165, 94-165, 95-165, 96-165, 97-165, 98-165, 99-165, 100-165, 101-165, 102-165, 103-165, 104-165, 105-165, 106-165, 107-165, 108-165, 109-165, 110-165, 111-165, 112-165, 113-165, 114-165, 115-165, 116-165, 117-165, 118-165, 119-165, 120-165, 121-165, 122-165, 123-165, 124-165, 125-165, 126-165, 127-165, 128-165, 129-165, 130-165, 131-165, 132-165, 133-165, 134-165, 135-165, 136-165, 137-165, 138-165, 139-165, 140-165, 141-165, 142-165, 143-165, 144-165, 145-165, 146-165, 147-165, 148-165, 149-165, 150-165, 151-165, 152-165, 153-165, 154-165, 155-165, 156-165, 157-165, 158-165, 159-165, 160-165, 161-165, 21-164, 22-164, 23-164, 24-164, 25-164, 26-164, 27-164, 28-164, 29-164, 30-164, 31-164, 32-164, 33-164, 34-164, 35-164, 36-164, 37-164, 38-164, 39-164, 40-164, 41-164, 42-164, 43-164, 44-164, 45-164, 46-164, 47-164, 48-164, 49-164, 50-164, 51-164, 52-164, 53-164, 54-164, 55-164, 56-164, 57-164, 58-164, 59-164, 60-164, 61-164, 62-164, 63-164, 64-164, 65-164, 66-164, 67-164, 68-164, 69-164, 70-164, 71-164, 72-164, 73-164, 74-164, 75-164, 76-164, 77-164, 78-164, 79-164, 80-164, 81-164, 82-164, 83-164, 84-164, 85-164, 86-164, 87-164, 88-164, 89-164, 90-164, 91-164, 92-164, 93-164, 94-164, 95-164, 96-164, 97-164, 98-164, 99-164, 100-164, 101-164, 102-164, 103-164, 104-164, 105-164, 106-164, 107-164, 108-164, 109-164, 110-164, 111-164, 112-164, 113-164, 114-164, 115-164, 116-164, 117-164, 118-164, 119-164, 120-164, 121-164, 122-164, 123-164, 124-164, 125-164, 126-164, 127-164, 128-164, 129-164, 130-164, 131-164, 132-164, 133-164, 134-164, 135-164, 136-164, 137-164, 138-164, 139-164, 140-164, 141-164, 142-164, 143-164, 144-164, 145-164, 146-164, 147-164, 148-164, 149-164, 150-164, 151-164, 152-164, 153-164, 154-164, 155-164, 156-164, 157-164, 158-164, 159-164, 160-164, 21-163, 22-163, 23-163, 24-163, 25-163, 26-163, 27-163, 28-163, 29-163, 30-163, 31-163, 32-163, 33-163, 34-163, 35-163, 36-163, 37-163, 38-163, 39-163, 40-163, 41-163, 42-163, 43-163, 44-163, 45-163, 46-163, 47-163, 48-163, 49-163, 50-163, 51-163, 52-163, 53-163, 54-163, 55-163, 56-163, 57-163, 58-163, 59-163, 60-163, 61-163, 62-163, 63-163, 64-163, 65-163, 66-163, 67-163, 68-163, 69-163, 70-163, 71-163, 72-163, 73-163, 74-163, 75-163, 76-163, 77-163, 78-163, 79-163, 80-163, 81-163, 82-163, 83-163, 84-163, 85-163, 86-163, 87-163, 88-163, 89-163, 90-163, 91-163, 92-163, 93-163, 94-163, 95-163, 96-163, 97-163, 98-163, 99-163, 100-163, 101-163, 102-163, 103-163, 104-163, 105-163, 106-163, 107-163, 108-163, 109-163, 110-163, 111-163, 112-163, 113-163, 114-163, 115-163, 116-163, 117-163, 118-163, 119-163, 120-163, 121-163, 122-163, 123-163, 124-163, 125-163, 126-163, 127-163, 128-163, 129-163, 130-163, 131-163, 132-163, 133-163, 134-163, 135-163, 136-163, 137-163, 138-163, 139-163, 140-163, 141-163, 142-163, 143-163, 144-163, 145-163, 146-163, 147-163, 148-163, 149-163, 150-163, 151-163, 152-163, 153-163, 154-163, 155-163, 156-163, 157-163, 158-163, 159-163, 21-162, 22-162, 23-162, 24-162, 25-162, 26-162, 27-162, 28-162, 29-162, 30-162, 31-162, 32-162, 33-162, 34-162, 35-162, 36-162, 37-162, 38-162, 39-162, 40-162, 41-162, 42-162, 43-162, 44-162, 45-162, 46-162, 47-162, 48-162, 49-162, 50-162, 51-162, 52-162, 53-162, 54-162, 55-162, 56-162, 57-162, 58-162, 59-162, 60-162, 61-162, 62-162, 63-162, 64-162, 65-162, 66-162, 67-162, 68-162, 69-162, 70-162, 71-162, 72-162, 73-162, 74-162, 75-162, 76-162, 77-162, 78-162, 79-162, 80-162, 81-162, 82-162, 83-162, 84-162, 85-162, 86-162, 87-162, 88-162, 89-162, 90-162, 91-162, 92-162, 93-162, 94-162, 95-162, 96-162, 97-162, 98-162, 99-162, 100-162, 101-162, 102-162, 103-162, 104-162, 105-162, 106-162, 107-162, 108-162, 109-162, 110-162, 111-162, 112-162, 113-162, 114-162, 115-162, 116-162, 117-162, 118-162, 119-162, 120-162, 121-162, 122-162, 123-162, 124-162, 125-162, 126-162, 127-162, 128-162, 129-162, 130-162, 131-162, 132-162, 133-162, 134-162, 135-162, 136-162, 137-162, 138-162, 139-162, 140-162, 141-162, 142-162, 143-162, 144-162, 145-162, 146-162, 147-162, 148-162, 149-162, 150-162, 151-162, 152-162, 153-162, 154-162, 155-162, 156-162, 157-162, 158-162, 21-161, 22-161, 23-161, 24-161, 25-161, 26-161, 27-161, 28-161, 29-161, 30-161, 31-161, 32-161, 33-161, 34-161, 35-161, 36-161, 37-161, 38-161, 39-161, 40-161, 41-161, 42-161, 43-161, 44-161, 45-161, 46-161, 47-161, 48-161, 49-161, 50-161, 51-161, 52-161, 53-161, 54-161, 55-161, 56-161, 57-161, 58-161, 59-161, 60-161, 61-161, 62-161, 63-161, 64-161, 65-161, 66-161, 67-161, 68-161, 69-161, 70-161, 71-161, 72-161, 73-161, 74-161, 75-161, 76-161, 77-161, 78-161, 79-161, 80-161, 81-161, 82-161, 83-161, 84-161, 85-161, 86-161, 87-161, 88-161, 89-161, 90-161, 91-161, 92-161, 93-161, 94-161, 95-161, 96-161, 97-161, 98-161, 99-161, 100-161, 101-161, 102-161, 103-161, 104-161, 105-161, 106-161, 107-161, 108-161, 109-161, 110-161, 111-161, 112-161, 113-161, 114-161, 115-161, 116-161, 117-161, 118-161, 119-161, 120-161, 121-161, 122-161, 123-161, 124-161, 125-161, 126-161, 127-161, 128-161, 129-161, 130-161, 131-161, 132-161, 133-161, 134-161, 135-161, 136-161, 137-161, 138-161, 139-161, 140-161, 141-161, 142-161, 143-161, 144-161, 145-161, 146-161, 147-161, 148-161, 149-161, 150-161, 151-161, 152-161, 153-161, 154-161, 155-161, 156-161, 157-161, 21-160, 22-160, 23-160, 24-160, 25-160, 26-160, 27-160, 28-160, 29-160, 30-160, 31-160, 32-160, 33-160, 34-160, 35-160, 36-160, 37-160, 38-160, 39-160, 40-160, 41-160, 42-160, 43-160, 44-160, 45-160, 46-160, 47-160, 48-160, 49-160, 50-160, 51-160, 52-160, 53-160, 54-160, 55-160, 56-160, 57-160, 58-160, 59-160, 60-160, 61-160, 62-160, 63-160, 64-160, 65-160, 66-160, 67-160, 68-160, 69-160, 70-160, 71-160, 72-160, 73-160, 74-160, 75-160, 76-160, 77-160, 78-160, 79-160, 80-160, 81-160, 82-160, 83-160, 84-160, 85-160, 86-160, 87-160, 88-160, 89-160, 90-160, 91-160, 92-160, 93-160, 94-160, 95-160, 96-160, 97-160, 98-160, 99-160, 100-160, 101-160, 102-160, 103-160, 104-160, 105-160, 106-160, 107-160, 108-160, 109-160, 110-160, 111-160, 112-160, 113-160, 114-160, 115-160, 116-160, 117-160, 118-160, 119-160, 120-160, 121-160, 122-160, 123-160, 124-160, 125-160, 126-160, 127-160, 128-160, 129-160, 130-160, 131-160, 132-160, 133-160, 134-160, 135-160, 136-160, 137-160, 138-160, 139-160, 140-160, 141-160, 142-160, 143-160, 144-160, 145-160, 146-160, 147-160, 148-160, 149-160, 150-160, 151-160, 152-160, 153-160, 154-160, 155-160, 156-160, 21-159, 22-159, 23-159, 24-159, 25-159, 26-159, 27-159, 28-159, 29-159, 30-159, 31-159, 32-159, 33-159, 34-159, 35-159, 36-159, 37-159, 38-159, 39-159, 40-159, 41-159, 42-159, 43-159, 44-159, 45-159, 46-159, 47-159, 48-159, 49-159, 50-159, 51-159, 52-159, 53-159, 54-159, 55-159, 56-159, 57-159, 58-159, 59-159, 60-159, 61-159, 62-159, 63-159, 64-159, 65-159, 66-159, 67-159, 68-159, 69-159, 70-159, 71-159, 72-159, 73-159, 74-159, 75-159, 76-159, 77-159, 78-159, 79-159, 80-159, 81-159, 82-159, 83-159, 84-159, 85-159, 86-159, 87-159, 88-159, 89-159, 90-159, 91-159, 92-159, 93-159, 94-159, 95-159, 96-159, 97-159, 98-159, 99-159, 100-159, 101-159, 102-159, 103-159, 104-159, 105-159, 106-159, 107-159, 108-159, 109-159, 110-159, 111-159, 112-159, 113-159, 114-159, 115-159, 116-159, 117-159, 118-159, 119-159, 120-159, 121-159, 122-159, 123-159, 124-159, 125-159, 126-159, 127-159, 128-159, 129-159, 130-159, 131-159, 132-159, 133-159, 134-159, 135-159, 136-159, 137-159, 138-159, 139-159, 140-159, 141-159, 142-159, 143-159, 144-159, 145-159, 146-159, 147-159, 148-159, 149-159, 150-159, 151-159, 152-159, 153-159, 154-159, 155-159, 21-158, 22-158, 23-158, 24-158, 25-158, 26-158, 27-158, 28-158, 29-158, 30-158, 31-158, 32-158, 33-158, 34-158, 35-158, 36-158, 37-158, 38-158, 39-158, 40-158, 41-158, 42-158, 43-158, 44-158, 45-158, 46-158, 47-158, 48-158, 49-158, 50-158, 51-158, 52-158, 53-158, 54-158, 55-158, 56-158, 57-158, 58-158, 59-158, 60-158, 61-158, 62-158, 63-158, 64-158, 65-158, 66-158, 67-158, 68-158, 69-158, 70-158, 71-158, 72-158, 73-158, 74-158, 75-158, 76-158, 77-158, 78-158, 79-158, 80-158, 81-158, 82-158, 83-158, 84-158, 85-158, 86-158, 87-158, 88-158, 89-158, 90-158, 91-158, 92-158, 93-158, 94-158, 95-158, 96-158, 97-158, 98-158, 99-158, 100-158, 101-158, 102-158, 103-158, 104-158, 105-158, 106-158, 107-158, 108-158, 109-158, 110-158, 111-158, 112-158, 113-158, 114-158, 115-158, 116-158, 117-158, 118-158, 119-158, 120-158, 121-158, 122-158, 123-158, 124-158, 125-158, 126-158, 127-158, 128-158, 129-158, 130-158, 131-158, 132-158, 133-158, 134-158, 135-158, 136-158, 137-158, 138-158, 139-158, 140-158, 141-158, 142-158, 143-158, 144-158, 145-158, 146-158, 147-158, 148-158, 149-158, 150-158, 151-158, 152-158, 153-158, 154-158, 21-157, 22-157, 23-157, 24-157, 25-157, 26-157, 27-157, 28-157, 29-157, 30-157, 31-157, 32-157, 33-157, 34-157, 35-157, 36-157, 37-157, 38-157, 39-157, 40-157, 41-157, 42-157, 43-157, 44-157, 45-157, 46-157, 47-157, 48-157, 49-157, 50-157, 51-157, 52-157, 53-157, 54-157, 55-157, 56-157, 57-157, 58-157, 59-157, 60-157, 61-157, 62-157, 63-157, 64-157, 65-157, 66-157, 67-157, 68-157, 69-157, 70-157, 71-157, 72-157, 73-157, 74-157, 75-157, 76-157, 77-157, 78-157, 79-157, 80-157, 81-157, 82-157, 83-157, 84-157, 85-157, 86-157, 87-157, 88-157, 89-157, 90-157, 91-157, 92-157, 93-157, 94-157, 95-157, 96-157, 97-157, 98-157, 99-157, 100-157, 101-157, 102-157, 103-157, 104-157, 105-157, 106-157, 107-157, 108-157, 109-157, 110-157, 111-157, 112-157, 113-157, 114-157, 115-157, 116-157, 117-157, 118-157, 119-157, 120-157, 121-157, 122-157, 123-157, 124-157, 125-157, 126-157, 127-157, 128-157, 129-157, 130-157, 131-157, 132-157, 133-157, 134-157, 135-157, 136-157, 137-157, 138-157, 139-157, 140-157, 141-157, 142-157, 143-157, 144-157, 145-157, 146-157, 147-157, 148-157, 149-157, 150-157, 151-157, 152-157, 153-157, 21-156, 22-156, 23-156, 24-156, 25-156, 26-156, 27-156, 28-156, 29-156, 30-156, 31-156, 32-156, 33-156, 34-156, 35-156, 36-156, 37-156, 38-156, 39-156, 40-156, 41-156, 42-156, 43-156, 44-156, 45-156, 46-156, 47-156, 48-156, 49-156, 50-156, 51-156, 52-156, 53-156, 54-156, 55-156, 56-156, 57-156, 58-156, 59-156, 60-156, 61-156, 62-156, 63-156, 64-156, 65-156, 66-156, 67-156, 68-156, 69-156, 70-156, 71-156, 72-156, 73-156, 74-156, 75-156, 76-156, 77-156, 78-156, 79-156, 80-156, 81-156, 82-156, 83-156, 84-156, 85-156, 86-156, 87-156, 88-156, 89-156, 90-156, 91-156, 92-156, 93-156, 94-156, 95-156, 96-156, 97-156, 98-156, 99-156, 100-156, 101-156, 102-156, 103-156, 104-156, 105-156, 106-156, 107-156, 108-156, 109-156, 110-156, 111-156, 112-156, 113-156, 114-156, 115-156, 116-156, 117-156, 118-156, 119-156, 120-156, 121-156, 122-156, 123-156, 124-156, 125-156, 126-156, 127-156, 128-156, 129-156, 130-156, 131-156, 132-156, 133-156, 134-156, 135-156, 136-156, 137-156, 138-156, 139-156, 140-156, 141-156, 142-156, 143-156, 144-156, 145-156, 146-156, 147-156, 148-156, 149-156, 150-156, 151-156, 152-156, 21-155, 22-155, 23-155, 24-155, 25-155, 26-155, 27-155, 28-155, 29-155, 30-155, 31-155, 32-155, 33-155, 34-155, 35-155, 36-155, 37-155, 38-155, 39-155, 40-155, 41-155, 42-155, 43-155, 44-155, 45-155, 46-155, 47-155, 48-155, 49-155, 50-155, 51-155, 52-155, 53-155, 54-155, 55-155, 56-155, 57-155, 58-155, 59-155, 60-155, 61-155, 62-155, 63-155, 64-155, 65-155, 66-155, 67-155, 68-155, 69-155, 70-155, 71-155, 72-155, 73-155, 74-155, 75-155, 76-155, 77-155, 78-155, 79-155, 80-155, 81-155, 82-155, 83-155, 84-155, 85-155, 86-155, 87-155, 88-155, 89-155, 90-155, 91-155, 92-155, 93-155, 94-155, 95-155, 96-155, 97-155, 98-155, 99-155, 100-155, 101-155, 102-155, 103-155, 104-155, 105-155, 106-155, 107-155, 108-155, 109-155, 110-155, 111-155, 112-155, 113-155, 114-155, 115-155, 116-155, 117-155, 118-155, 119-155, 120-155, 121-155, 122-155, 123-155, 124-155, 125-155, 126-155, 127-155, 128-155, 129-155, 130-155, 131-155, 132-155, 133-155, 134-155, 135-155, 136-155, 137-155, 138-155, 139-155, 140-155, 141-155, 142-155, 143-155, 144-155, 145-155, 146-155, 147-155, 148-155, 149-155, 150-155, 151-155, 21-154, 22-154, 23-154, 24-154, 25-154, 26-154, 27-154, 28-154, 29-154, 30-154, 31-154, 32-154, 33-154, 34-154, 35-154, 36-154, 37-154, 38-154, 39-154, 40-154, 41-154, 42-154, 43-154, 44-154, 45-154, 46-154, 47-154, 48-154, 49-154, 50-154, 51-154, 52-154, 53-154, 54-154, 55-154, 56-154, 57-154, 58-154, 59-154, 60-154, 61-154, 62-154, 63-154, 64-154, 65-154, 66-154, 67-154, 68-154, 69-154, 70-154, 71-154, 72-154, 73-154, 74-154, 75-154, 76-154, 77-154, 78-154, 79-154, 80-154, 81-154, 82-154, 83-154, 84-154, 85-154, 86-154, 87-154, 88-154, 89-154, 90-154, 91-154, 92-154, 93-154, 94-154, 95-154, 96-154, 97-154, 98-154, 99-154, 100-154, 101-154, 102-154, 103-154, 104-154, 105-154, 106-154, 107-154, 108-154, 109-154, 110-154, 111-154, 112-154, 113-154, 114-154, 115-154, 116-154, 117-154, 118-154, 119-154, 120-154, 121-154, 122-154, 123-154, 124-154, 125-154, 126-154, 127-154, 128-154, 129-154, 130-154, 131-154, 132-154, 133-154, 134-154, 135-154, 136-154, 137-154, 138-154, 139-154, 140-154, 141-154, 142-154, 143-154, 144-154, 145-154, 146-154, 147-154, 148-154, 149-154, 150-154, 21-153, 22-153, 23-153, 24-153, 25-153, 26-153, 27-153, 28-153, 29-153, 30-153, 31-153, 32-153, 33-153, 34-153, 35-153, 36-153, 37-153, 38-153, 39-153, 40-153, 41-153, 42-153, 43-153, 44-153, 45-153, 46-153, 47-153, 48-153, 49-153, 50-153, 51-153, 52-153, 53-153, 54-153, 55-153, 56-153, 57-153, 58-153, 59-153, 60-153, 61-153, 62-153, 63-153, 64-153, 65-153, 66-153, 67-153, 68-153, 69-153, 70-153, 71-153, 72-153, 73-153, 74-153, 75-153, 76-153, 77-153, 78-153, 79-153, 80-153, 81-153, 82-153, 83-153, 84-153, 85-153, 86-153, 87-153, 88-153, 89-153, 90-153, 91-153, 92-153, 93-153, 94-153, 95-153, 96-153, 97-153, 98-153, 99-153, 100-153, 101-153, 102-153, 103-153, 104-153, 105-153, 106-153, 107-153, 108-153, 109-153, 110-153, 111-153, 112-153, 113-153, 114-153, 115-153, 116-153, 117-153, 118-153, 119-153, 120-153, 121-153, 122-153, 123-153, 124-153, 125-153, 126-153, 127-153, 128-153, 129-153, 130-153, 131-153, 132-153, 133-153, 134-153, 135-153, 136-153, 137-153, 138-153, 139-153, 140-153, 141-153, 142-153, 143-153, 144-153, 145-153, 146-153, 147-153, 148-153, 149-153, 21-152, 22-152, 23-152, 24-152, 25-152, 26-152, 27-152, 28-152, 29-152, 30-152, 31-152, 32-152, 33-152, 34-152, 35-152, 36-152, 37-152, 38-152, 39-152, 40-152, 41-152, 42-152, 43-152, 44-152, 45-152, 46-152, 47-152, 48-152, 49-152, 50-152, 51-152, 52-152, 53-152, 54-152, 55-152, 56-152, 57-152, 58-152, 59-152, 60-152, 61-152, 62-152, 63-152, 64-152, 65-152, 66-152, 67-152, 68-152, 69-152, 70-152, 71-152, 72-152, 73-152, 74-152, 75-152, 76-152, 77-152, 78-152, 79-152, 80-152, 81-152, 82-152, 83-152, 84-152, 85-152, 86-152, 87-152, 88-152, 89-152, 90-152, 91-152, 92-152, 93-152, 94-152, 95-152, 96-152, 97-152, 98-152, 99-152, 100-152, 101-152, 102-152, 103-152, 104-152, 105-152, 106-152, 107-152, 108-152, 109-152, 110-152, 111-152, 112-152, 113-152, 114-152, 115-152, 116-152, 117-152, 118-152, 119-152, 120-152, 121-152, 122-152, 123-152, 124-152, 125-152, 126-152, 127-152, 128-152, 129-152, 130-152, 131-152, 132-152, 133-152, 134-152, 135-152, 136-152, 137-152, 138-152, 139-152, 140-152, 141-152, 142-152, 143-152, 144-152, 145-152, 146-152, 147-152, 148-152, 21-151, 22-151, 23-151, 24-151, 25-151, 26-151, 27-151, 28-151, 29-151, 30-151, 31-151, 32-151, 33-151, 34-151, 35-151, 36-151, 37-151, 38-151, 39-151, 40-151, 41-151, 42-151, 43-151, 44-151, 45-151, 46-151, 47-151, 48-151, 49-151, 50-151, 51-151, 52-151, 53-151, 54-151, 55-151, 56-151, 57-151, 58-151, 59-151, 60-151, 61-151, 62-151, 63-151, 64-151, 65-151, 66-151, 67-151, 68-151, 69-151, 70-151, 71-151, 72-151, 73-151, 74-151, 75-151, 76-151, 77-151, 78-151, 79-151, 80-151, 81-151, 82-151, 83-151, 84-151, 85-151, 86-151, 87-151, 88-151, 89-151, 90-151, 91-151, 92-151, 93-151, 94-151, 95-151, 96-151, 97-151, 98-151, 99-151, 100-151, 101-151, 102-151, 103-151, 104-151, 105-151, 106-151, 107-151, 108-151, 109-151, 110-151, 111-151, 112-151, 113-151, 114-151, 115-151, 116-151, 117-151, 118-151, 119-151, 120-151, 121-151, 122-151, 123-151, 124-151, 125-151, 126-151, 127-151, 128-151, 129-151, 130-151, 131-151, 132-151, 133-151, 134-151, 135-151, 136-151, 137-151, 138-151, 139-151, 140-151, 141-151, 142-151, 143-151, 144-151, 145-151, 146-151, 147-151, 21-150, 22-150, 23-150, 24-150, 25-150, 26-150, 27-150, 28-150, 29-150, 30-150, 31-150, 32-150, 33-150, 34-150, 35-150, 36-150, 37-150, 38-150, 39-150, 40-150, 41-150, 42-150, 43-150, 44-150, 45-150, 46-150, 47-150, 48-150, 49-150, 50-150, 51-150, 52-150, 53-150, 54-150, 55-150, 56-150, 57-150, 58-150, 59-150, 60-150, 61-150, 62-150, 63-150, 64-150, 65-150, 66-150, 67-150, 68-150, 69-150, 70-150, 71-150, 72-150, 73-150, 74-150, 75-150, 76-150, 77-150, 78-150, 79-150, 80-150, 81-150, 82-150, 83-150, 84-150, 85-150, 86-150, 87-150, 88-150, 89-150, 90-150, 91-150, 92-150, 93-150, 94-150, 95-150, 96-150, 97-150, 98-150, 99-150, 100-150, 101-150, 102-150, 103-150, 104-150, 105-150, 106-150, 107-150, 108-150, 109-150, 110-150, 111-150, 112-150, 113-150, 114-150, 115-150, 116-150, 117-150, 118-150, 119-150, 120-150, 121-150, 122-150, 123-150, 124-150, 125-150, 126-150, 127-150, 128-150, 129-150, 130-150, 131-150, 132-150, 133-150, 134-150, 135-150, 136-150, 137-150, 138-150, 139-150, 140-150, 141-150, 142-150, 143-150, 144-150, 145-150, 146-150, 21-149, 22-149, 23-149, 24-149, 25-149, 26-149, 27-149, 28-149, 29-149, 30-149, 31-149, 32-149, 33-149, 34-149, 35-149, 36-149, 37-149, 38-149, 39-149, 40-149, 41-149, 42-149, 43-149, 44-149, 45-149, 46-149, 47-149, 48-149, 49-149, 50-149, 51-149, 52-149, 53-149, 54-149, 55-149, 56-149, 57-149, 58-149, 59-149, 60-149, 61-149, 62-149, 63-149, 64-149, 65-149, 66-149, 67-149, 68-149, 69-149, 70-149, 71-149, 72-149, 73-149, 74-149, 75-149, 76-149, 77-149, 78-149, 79-149, 80-149, 81-149, 82-149, 83-149, 84-149, 85-149, 86-149, 87-149, 88-149, 89-149, 90-149, 91-149, 92-149, 93-149, 94-149, 95-149, 96-149, 97-149, 98-149, 99-149, 100-149, 101-149, 102-149, 103-149, 104-149, 105-149, 106-149, 107-149, 108-149, 109-149, 110-149, 111-149, 112-149, 113-149, 114-149, 115-149, 116-149, 117-149, 118-149, 119-149, 120-149, 121-149, 122-149, 123-149, 124-149, 125-149, 126-149, 127-149, 128-149, 129-149, 130-149, 131-149, 132-149, 133-149, 134-149, 135-149, 136-149, 137-149, 138-149, 139-149, 140-149, 141-149, 142-149, 143-149, 144-149, 145-149, 21-148, 22-148, 23-148, 24-148, 25-148, 26-148, 27-148, 28-148, 29-148, 30-148, 31-148, 32-148, 33-148, 34-148, 35-148, 36-148, 37-148, 38-148, 39-148, 40-148, 41-148, 42-148, 43-148, 44-148, 45-148, 46-148, 47-148, 48-148, 49-148, 50-148, 51-148, 52-148, 53-148, 54-148, 55-148, 56-148, 57-148, 58-148, 59-148, 60-148, 61-148, 62-148, 63-148, 64-148, 65-148, 66-148, 67-148, 68-148, 69-148, 70-148, 71-148, 72-148, 73-148, 74-148, 75-148, 76-148, 77-148, 78-148, 79-148, 80-148, 81-148, 82-148, 83-148, 84-148, 85-148, 86-148, 87-148, 88-148, 89-148, 90-148, 91-148, 92-148, 93-148, 94-148, 95-148, 96-148, 97-148, 98-148, 99-148, 100-148, 101-148, 102-148, 103-148, 104-148, 105-148, 106-148, 107-148, 108-148, 109-148, 110-148, 111-148, 112-148, 113-148, 114-148, 115-148, 116-148, 117-148, 118-148, 119-148, 120-148, 121-148, 122-148, 123-148, 124-148, 125-148, 126-148, 127-148, 128-148, 129-148, 130-148, 131-148, 132-148, 133-148, 134-148, 135-148, 136-148, 137-148, 138-148, 139-148, 140-148, 141-148, 142-148, 143-148, 144-148, 21-147, 22-147, 23-147, 24-147, 25-147, 26-147, 27-147, 28-147, 29-147, 30-147, 31-147, 32-147, 33-147, 34-147, 35-147, 36-147, 37-147, 38-147, 39-147, 40-147, 41-147, 42-147, 43-147, 44-147, 45-147, 46-147, 47-147, 48-147, 49-147, 50-147, 51-147, 52-147, 53-147, 54-147, 55-147, 56-147, 57-147, 58-147, 59-147, 60-147, 61-147, 62-147, 63-147, 64-147, 65-147, 66-147, 67-147, 68-147, 69-147, 70-147, 71-147, 72-147, 73-147, 74-147, 75-147, 76-147, 77-147, 78-147, 79-147, 80-147, 81-147, 82-147, 83-147, 84-147, 85-147, 86-147, 87-147, 88-147, 89-147, 90-147, 91-147, 92-147, 93-147, 94-147, 95-147, 96-147, 97-147, 98-147, 99-147, 100-147, 101-147, 102-147, 103-147, 104-147, 105-147, 106-147, 107-147, 108-147, 109-147, 110-147, 111-147, 112-147, 113-147, 114-147, 115-147, 116-147, 117-147, 118-147, 119-147, 120-147, 121-147, 122-147, 123-147, 124-147, 125-147, 126-147, 127-147, 128-147, 129-147, 130-147, 131-147, 132-147, 133-147, 134-147, 135-147, 136-147, 137-147, 138-147, 139-147, 140-147, 141-147, 142-147, 143-147, 21-146, 22-146, 23-146, 24-146, 25-146, 26-146, 27-146, 28-146, 29-146, 30-146, 31-146, 32-146, 33-146, 34-146, 35-146, 36-146, 37-146, 38-146, 39-146, 40-146, 41-146, 42-146, 43-146, 44-146, 45-146, 46-146, 47-146, 48-146, 49-146, 50-146, 51-146, 52-146, 53-146, 54-146, 55-146, 56-146, 57-146, 58-146, 59-146, 60-146, 61-146, 62-146, 63-146, 64-146, 65-146, 66-146, 67-146, 68-146, 69-146, 70-146, 71-146, 72-146, 73-146, 74-146, 75-146, 76-146, 77-146, 78-146, 79-146, 80-146, 81-146, 82-146, 83-146, 84-146, 85-146, 86-146, 87-146, 88-146, 89-146, 90-146, 91-146, 92-146, 93-146, 94-146, 95-146, 96-146, 97-146, 98-146, 99-146, 100-146, 101-146, 102-146, 103-146, 104-146, 105-146, 106-146, 107-146, 108-146, 109-146, 110-146, 111-146, 112-146, 113-146, 114-146, 115-146, 116-146, 117-146, 118-146, 119-146, 120-146, 121-146, 122-146, 123-146, 124-146, 125-146, 126-146, 127-146, 128-146, 129-146, 130-146, 131-146, 132-146, 133-146, 134-146, 135-146, 136-146, 137-146, 138-146, 139-146, 140-146, 141-146, 142-146, 21-145, 22-145, 23-145, 24-145, 25-145, 26-145, 27-145, 28-145, 29-145, 30-145, 31-145, 32-145, 33-145, 34-145, 35-145, 36-145, 37-145, 38-145, 39-145, 40-145, 41-145, 42-145, 43-145, 44-145, 45-145, 46-145, 47-145, 48-145, 49-145, 50-145, 51-145, 52-145, 53-145, 54-145, 55-145, 56-145, 57-145, 58-145, 59-145, 60-145, 61-145, 62-145, 63-145, 64-145, 65-145, 66-145, 67-145, 68-145, 69-145, 70-145, 71-145, 72-145, 73-145, 74-145, 75-145, 76-145, 77-145, 78-145, 79-145, 80-145, 81-145, 82-145, 83-145, 84-145, 85-145, 86-145, 87-145, 88-145, 89-145, 90-145, 91-145, 92-145, 93-145, 94-145, 95-145, 96-145, 97-145, 98-145, 99-145, 100-145, 101-145, 102-145, 103-145, 104-145, 105-145, 106-145, 107-145, 108-145, 109-145, 110-145, 111-145, 112-145, 113-145, 114-145, 115-145, 116-145, 117-145, 118-145, 119-145, 120-145, 121-145, 122-145, 123-145, 124-145, 125-145, 126-145, 127-145, 128-145, 129-145, 130-145, 131-145, 132-145, 133-145, 134-145, 135-145, 136-145, 137-145, 138-145, 139-145, 140-145, 141-145, 21-144, 22-144, 23-144, 24-144, 25-144, 26-144, 27-144, 28-144, 29-144, 30-144, 31-144, 32-144, 33-144, 34-144, 35-144, 36-144, 37-144, 38-144, 39-144, 40-144, 41-144, 42-144, 43-144, 44-144, 45-144, 46-144, 47-144, 48-144, 49-144, 50-144, 51-144, 52-144, 53-144, 54-144, 55-144, 56-144, 57-144, 58-144, 59-144, 60-144, 61-144, 62-144, 63-144, 64-144, 65-144, 66-144, 67-144, 68-144, 69-144, 70-144, 71-144, 72-144, 73-144, 74-144, 75-144, 76-144, 77-144, 78-144, 79-144, 80-144, 81-144, 82-144, 83-144, 84-144, 85-144, 86-144, 87-144, 88-144, 89-144, 90-144, 91-144, 92-144, 93-144, 94-144, 95-144, 96-144, 97-144, 98-144, 99-144, 100-144, 101-144, 102-144, 103-144, 104-144, 105-144, 106-144, 107-144, 108-144, 109-144, 110-144, 111-144, 112-144, 113-144, 114-144, 115-144, 116-144, 117-144, 118-144, 119-144, 120-144, 121-144, 122-144, 123-144, 124-144, 125-144, 126-144, 127-144, 128-144, 129-144, 130-144, 131-144, 132-144, 133-144, 134-144, 135-144, 136-144, 137-144, 138-144, 139-144, 140-144, 21-143, 22-143, 23-143, 24-143, 25-143, 26-143, 27-143, 28-143, 29-143, 30-143, 31-143, 32-143, 33-143, 34-143, 35-143, 36-143, 37-143, 38-143, 39-143, 40-143, 41-143, 42-143, 43-143, 44-143, 45-143, 46-143, 47-143, 48-143, 49-143, 50-143, 51-143, 52-143, 53-143, 54-143, 55-143, 56-143, 57-143, 58-143, 59-143, 60-143, 61-143, 62-143, 63-143, 64-143, 65-143, 66-143, 67-143, 68-143, 69-143, 70-143, 71-143, 72-143, 73-143, 74-143, 75-143, 76-143, 77-143, 78-143, 79-143, 80-143, 81-143, 82-143, 83-143, 84-143, 85-143, 86-143, 87-143, 88-143, 89-143, 90-143, 91-143, 92-143, 93-143, 94-143, 95-143, 96-143, 97-143, 98-143, 99-143, 100-143, 101-143, 102-143, 103-143, 104-143, 105-143, 106-143, 107-143, 108-143, 109-143, 110-143, 111-143, 112-143, 113-143, 114-143, 115-143, 116-143, 117-143, 118-143, 119-143, 120-143, 121-143, 122-143, 123-143, 124-143, 125-143, 126-143, 127-143, 128-143, 129-143, 130-143, 131-143, 132-143, 133-143, 134-143, 135-143, 136-143, 137-143, 138-143, 139-143, 21-142, 22-142, 23-142, 24-142, 25-142, 26-142, 27-142, 28-142, 29-142, 30-142, 31-142, 32-142, 33-142, 34-142, 35-142, 36-142, 37-142, 38-142, 39-142, 40-142, 41-142, 42-142, 43-142, 44-142, 45-142, 46-142, 47-142, 48-142, 49-142, 50-142, 51-142, 52-142, 53-142, 54-142, 55-142, 56-142, 57-142, 58-142, 59-142, 60-142, 61-142, 62-142, 63-142, 64-142, 65-142, 66-142, 67-142, 68-142, 69-142, 70-142, 71-142, 72-142, 73-142, 74-142, 75-142, 76-142, 77-142, 78-142, 79-142, 80-142, 81-142, 82-142, 83-142, 84-142, 85-142, 86-142, 87-142, 88-142, 89-142, 90-142, 91-142, 92-142, 93-142, 94-142, 95-142, 96-142, 97-142, 98-142, 99-142, 100-142, 101-142, 102-142, 103-142, 104-142, 105-142, 106-142, 107-142, 108-142, 109-142, 110-142, 111-142, 112-142, 113-142, 114-142, 115-142, 116-142, 117-142, 118-142, 119-142, 120-142, 121-142, 122-142, 123-142, 124-142, 125-142, 126-142, 127-142, 128-142, 129-142, 130-142, 131-142, 132-142, 133-142, 134-142, 135-142, 136-142, 137-142, 138-142, 21-141, 22-141, 23-141, 24-141, 25-141, 26-141, 27-141, 28-141, 29-141, 30-141, 31-141, 32-141, 33-141, 34-141, 35-141, 36-141, 37-141, 38-141, 39-141, 40-141, 41-141, 42-141, 43-141, 44-141, 45-141, 46-141, 47-141, 48-141, 49-141, 50-141, 51-141, 52-141, 53-141, 54-141, 55-141, 56-141, 57-141, 58-141, 59-141, 60-141, 61-141, 62-141, 63-141, 64-141, 65-141, 66-141, 67-141, 68-141, 69-141, 70-141, 71-141, 72-141, 73-141, 74-141, 75-141, 76-141, 77-141, 78-141, 79-141, 80-141, 81-141, 82-141, 83-141, 84-141, 85-141, 86-141, 87-141, 88-141, 89-141, 90-141, 91-141, 92-141, 93-141, 94-141, 95-141, 96-141, 97-141, 98-141, 99-141, 100-141, 101-141, 102-141, 103-141, 104-141, 105-141, 106-141, 107-141, 108-141, 109-141, 110-141, 111-141, 112-141, 113-141, 114-141, 115-141, 116-141, 117-141, 118-141, 119-141, 120-141, 121-141, 122-141, 123-141, 124-141, 125-141, 126-141, 127-141, 128-141, 129-141, 130-141, 131-141, 132-141, 133-141, 134-141, 135-141, 136-141, 137-141, 21-140, 22-140, 23-140, 24-140, 25-140, 26-140, 27-140, 28-140, 29-140, 30-140, 31-140, 32-140, 33-140, 34-140, 35-140, 36-140, 37-140, 38-140, 39-140, 40-140, 41-140, 42-140, 43-140, 44-140, 45-140, 46-140, 47-140, 48-140, 49-140, 50-140, 51-140, 52-140, 53-140, 54-140, 55-140, 56-140, 57-140, 58-140, 59-140, 60-140, 61-140, 62-140, 63-140, 64-140, 65-140, 66-140, 67-140, 68-140, 69-140, 70-140, 71-140, 72-140, 73-140, 74-140, 75-140, 76-140, 77-140, 78-140, 79-140, 80-140, 81-140, 82-140, 83-140, 84-140, 85-140, 86-140, 87-140, 88-140, 89-140, 90-140, 91-140, 92-140, 93-140, 94-140, 95-140, 96-140, 97-140, 98-140, 99-140, 100-140, 101-140, 102-140, 103-140, 104-140, 105-140, 106-140, 107-140, 108-140, 109-140, 110-140, 111-140, 112-140, 113-140, 114-140, 115-140, 116-140, 117-140, 118-140, 119-140, 120-140, 121-140, 122-140, 123-140, 124-140, 125-140, 126-140, 127-140, 128-140, 129-140, 130-140, 131-140, 132-140, 133-140, 134-140, 135-140, 136-140, 21-139, 22-139, 23-139, 24-139, 25-139, 26-139, 27-139, 28-139, 29-139, 30-139, 31-139, 32-139, 33-139, 34-139, 35-139, 36-139, 37-139, 38-139, 39-139, 40-139, 41-139, 42-139, 43-139, 44-139, 45-139, 46-139, 47-139, 48-139, 49-139, 50-139, 51-139, 52-139, 53-139, 54-139, 55-139, 56-139, 57-139, 58-139, 59-139, 60-139, 61-139, 62-139, 63-139, 64-139, 65-139, 66-139, 67-139, 68-139, 69-139, 70-139, 71-139, 72-139, 73-139, 74-139, 75-139, 76-139, 77-139, 78-139, 79-139, 80-139, 81-139, 82-139, 83-139, 84-139, 85-139, 86-139, 87-139, 88-139, 89-139, 90-139, 91-139, 92-139, 93-139, 94-139, 95-139, 96-139, 97-139, 98-139, 99-139, 100-139, 101-139, 102-139, 103-139, 104-139, 105-139, 106-139, 107-139, 108-139, 109-139, 110-139, 111-139, 112-139, 113-139, 114-139, 115-139, 116-139, 117-139, 118-139, 119-139, 120-139, 121-139, 122-139, 123-139, 124-139, 125-139, 126-139, 127-139, 128-139, 129-139, 130-139, 131-139, 132-139, 133-139, 134-139, 135-139, 21-138, 22-138, 23-138, 24-138, 25-138, 26-138, 27-138, 28-138, 29-138, 30-138, 31-138, 32-138, 33-138, 34-138, 35-138, 36-138, 37-138, 38-138, 39-138, 40-138, 41-138, 42-138, 43-138, 44-138, 45-138, 46-138, 47-138, 48-138, 49-138, 50-138, 51-138, 52-138, 53-138, 54-138, 55-138, 56-138, 57-138, 58-138, 59-138, 60-138, 61-138, 62-138, 63-138, 64-138, 65-138, 66-138, 67-138, 68-138, 69-138, 70-138, 71-138, 72-138, 73-138, 74-138, 75-138, 76-138, 77-138, 78-138, 79-138, 80-138, 81-138, 82-138, 83-138, 84-138, 85-138, 86-138, 87-138, 88-138, 89-138, 90-138, 91-138, 92-138, 93-138, 94-138, 95-138, 96-138, 97-138, 98-138, 99-138, 100-138, 101-138, 102-138, 103-138, 104-138, 105-138, 106-138, 107-138, 108-138, 109-138, 110-138, 111-138, 112-138, 113-138, 114-138, 115-138, 116-138, 117-138, 118-138, 119-138, 120-138, 121-138, 122-138, 123-138, 124-138, 125-138, 126-138, 127-138, 128-138, 129-138, 130-138, 131-138, 132-138, 133-138, 134-138, 21-137, 22-137, 23-137, 24-137, 25-137, 26-137, 27-137, 28-137, 29-137, 30-137, 31-137, 32-137, 33-137, 34-137, 35-137, 36-137, 37-137, 38-137, 39-137, 40-137, 41-137, 42-137, 43-137, 44-137, 45-137, 46-137, 47-137, 48-137, 49-137, 50-137, 51-137, 52-137, 53-137, 54-137, 55-137, 56-137, 57-137, 58-137, 59-137, 60-137, 61-137, 62-137, 63-137, 64-137, 65-137, 66-137, 67-137, 68-137, 69-137, 70-137, 71-137, 72-137, 73-137, 74-137, 75-137, 76-137, 77-137, 78-137, 79-137, 80-137, 81-137, 82-137, 83-137, 84-137, 85-137, 86-137, 87-137, 88-137, 89-137, 90-137, 91-137, 92-137, 93-137, 94-137, 95-137, 96-137, 97-137, 98-137, 99-137, 100-137, 101-137, 102-137, 103-137, 104-137, 105-137, 106-137, 107-137, 108-137, 109-137, 110-137, 111-137, 112-137, 113-137, 114-137, 115-137, 116-137, 117-137, 118-137, 119-137, 120-137, 121-137, 122-137, 123-137, 124-137, 125-137, 126-137, 127-137, 128-137, 129-137, 130-137, 131-137, 132-137, 133-137, 21-136, 22-136, 23-136, 24-136, 25-136, 26-136, 27-136, 28-136, 29-136, 30-136, 31-136, 32-136, 33-136, 34-136, 35-136, 36-136, 37-136, 38-136, 39-136, 40-136, 41-136, 42-136, 43-136, 44-136, 45-136, 46-136, 47-136, 48-136, 49-136, 50-136, 51-136, 52-136, 53-136, 54-136, 55-136, 56-136, 57-136, 58-136, 59-136, 60-136, 61-136, 62-136, 63-136, 64-136, 65-136, 66-136, 67-136, 68-136, 69-136, 70-136, 71-136, 72-136, 73-136, 74-136, 75-136, 76-136, 77-136, 78-136, 79-136, 80-136, 81-136, 82-136, 83-136, 84-136, 85-136, 86-136, 87-136, 88-136, 89-136, 90-136, 91-136, 92-136, 93-136, 94-136, 95-136, 96-136, 97-136, 98-136, 99-136, 100-136, 101-136, 102-136, 103-136, 104-136, 105-136, 106-136, 107-136, 108-136, 109-136, 110-136, 111-136, 112-136, 113-136, 114-136, 115-136, 116-136, 117-136, 118-136, 119-136, 120-136, 121-136, 122-136, 123-136, 124-136, 125-136, 126-136, 127-136, 128-136, 129-136, 130-136, 131-136, 132-136, 21-135, 22-135, 23-135, 24-135, 25-135, 26-135, 27-135, 28-135, 29-135, 30-135, 31-135, 32-135, 33-135, 34-135, 35-135, 36-135, 37-135, 38-135, 39-135, 40-135, 41-135, 42-135, 43-135, 44-135, 45-135, 46-135, 47-135, 48-135, 49-135, 50-135, 51-135, 52-135, 53-135, 54-135, 55-135, 56-135, 57-135, 58-135, 59-135, 60-135, 61-135, 62-135, 63-135, 64-135, 65-135, 66-135, 67-135, 68-135, 69-135, 70-135, 71-135, 72-135, 73-135, 74-135, 75-135, 76-135, 77-135, 78-135, 79-135, 80-135, 81-135, 82-135, 83-135, 84-135, 85-135, 86-135, 87-135, 88-135, 89-135, 90-135, 91-135, 92-135, 93-135, 94-135, 95-135, 96-135, 97-135, 98-135, 99-135, 100-135, 101-135, 102-135, 103-135, 104-135, 105-135, 106-135, 107-135, 108-135, 109-135, 110-135, 111-135, 112-135, 113-135, 114-135, 115-135, 116-135, 117-135, 118-135, 119-135, 120-135, 121-135, 122-135, 123-135, 124-135, 125-135, 126-135, 127-135, 128-135, 129-135, 130-135, 131-135, 21-134, 22-134, 23-134, 24-134, 25-134, 26-134, 27-134, 28-134, 29-134, 30-134, 31-134, 32-134, 33-134, 34-134, 35-134, 36-134, 37-134, 38-134, 39-134, 40-134, 41-134, 42-134, 43-134, 44-134, 45-134, 46-134, 47-134, 48-134, 49-134, 50-134, 51-134, 52-134, 53-134, 54-134, 55-134, 56-134, 57-134, 58-134, 59-134, 60-134, 61-134, 62-134, 63-134, 64-134, 65-134, 66-134, 67-134, 68-134, 69-134, 70-134, 71-134, 72-134, 73-134, 74-134, 75-134, 76-134, 77-134, 78-134, 79-134, 80-134, 81-134, 82-134, 83-134, 84-134, 85-134, 86-134, 87-134, 88-134, 89-134, 90-134, 91-134, 92-134, 93-134, 94-134, 95-134, 96-134, 97-134, 98-134, 99-134, 100-134, 101-134, 102-134, 103-134, 104-134, 105-134, 106-134, 107-134, 108-134, 109-134, 110-134, 111-134, 112-134, 113-134, 114-134, 115-134, 116-134, 117-134, 118-134, 119-134, 120-134, 121-134, 122-134, 123-134, 124-134, 125-134, 126-134, 127-134, 128-134, 129-134, 130-134, 21-133, 22-133, 23-133, 24-133, 25-133, 26-133, 27-133, 28-133, 29-133, 30-133, 31-133, 32-133, 33-133, 34-133, 35-133, 36-133, 37-133, 38-133, 39-133, 40-133, 41-133, 42-133, 43-133, 44-133, 45-133, 46-133, 47-133, 48-133, 49-133, 50-133, 51-133, 52-133, 53-133, 54-133, 55-133, 56-133, 57-133, 58-133, 59-133, 60-133, 61-133, 62-133, 63-133, 64-133, 65-133, 66-133, 67-133, 68-133, 69-133, 70-133, 71-133, 72-133, 73-133, 74-133, 75-133, 76-133, 77-133, 78-133, 79-133, 80-133, 81-133, 82-133, 83-133, 84-133, 85-133, 86-133, 87-133, 88-133, 89-133, 90-133, 91-133, 92-133, 93-133, 94-133, 95-133, 96-133, 97-133, 98-133, 99-133, 100-133, 101-133, 102-133, 103-133, 104-133, 105-133, 106-133, 107-133, 108-133, 109-133, 110-133, 111-133, 112-133, 113-133, 114-133, 115-133, 116-133, 117-133, 118-133, 119-133, 120-133, 121-133, 122-133, 123-133, 124-133, 125-133, 126-133, 127-133, 128-133, 129-133, 21-132, 22-132, 23-132, 24-132, 25-132, 26-132, 27-132, 28-132, 29-132, 30-132, 31-132, 32-132, 33-132, 34-132, 35-132, 36-132, 37-132, 38-132, 39-132, 40-132, 41-132, 42-132, 43-132, 44-132, 45-132, 46-132, 47-132, 48-132, 49-132, 50-132, 51-132, 52-132, 53-132, 54-132, 55-132, 56-132, 57-132, 58-132, 59-132, 60-132, 61-132, 62-132, 63-132, 64-132, 65-132, 66-132, 67-132, 68-132, 69-132, 70-132, 71-132, 72-132, 73-132, 74-132, 75-132, 76-132, 77-132, 78-132, 79-132, 80-132, 81-132, 82-132, 83-132, 84-132, 85-132, 86-132, 87-132, 88-132, 89-132, 90-132, 91-132, 92-132, 93-132, 94-132, 95-132, 96-132, 97-132, 98-132, 99-132, 100-132, 101-132, 102-132, 103-132, 104-132, 105-132, 106-132, 107-132, 108-132, 109-132, 110-132, 111-132, 112-132, 113-132, 114-132, 115-132, 116-132, 117-132, 118-132, 119-132, 120-132, 121-132, 122-132, 123-132, 124-132, 125-132, 126-132, 127-132, 128-132, 21-131, 22-131, 23-131, 24-131, 25-131, 26-131, 27-131, 28-131, 29-131, 30-131, 31-131, 32-131, 33-131, 34-131, 35-131, 36-131, 37-131, 38-131, 39-131, 40-131, 41-131, 42-131, 43-131, 44-131, 45-131, 46-131, 47-131, 48-131, 49-131, 50-131, 51-131, 52-131, 53-131, 54-131, 55-131, 56-131, 57-131, 58-131, 59-131, 60-131, 61-131, 62-131, 63-131, 64-131, 65-131, 66-131, 67-131, 68-131, 69-131, 70-131, 71-131, 72-131, 73-131, 74-131, 75-131, 76-131, 77-131, 78-131, 79-131, 80-131, 81-131, 82-131, 83-131, 84-131, 85-131, 86-131, 87-131, 88-131, 89-131, 90-131, 91-131, 92-131, 93-131, 94-131, 95-131, 96-131, 97-131, 98-131, 99-131, 100-131, 101-131, 102-131, 103-131, 104-131, 105-131, 106-131, 107-131, 108-131, 109-131, 110-131, 111-131, 112-131, 113-131, 114-131, 115-131, 116-131, 117-131, 118-131, 119-131, 120-131, 121-131, 122-131, 123-131, 124-131, 125-131, 126-131, 127-131, 21-130, 22-130, 23-130, 24-130, 25-130, 26-130, 27-130, 28-130, 29-130, 30-130, 31-130, 32-130, 33-130, 34-130, 35-130, 36-130, 37-130, 38-130, 39-130, 40-130, 41-130, 42-130, 43-130, 44-130, 45-130, 46-130, 47-130, 48-130, 49-130, 50-130, 51-130, 52-130, 53-130, 54-130, 55-130, 56-130, 57-130, 58-130, 59-130, 60-130, 61-130, 62-130, 63-130, 64-130, 65-130, 66-130, 67-130, 68-130, 69-130, 70-130, 71-130, 72-130, 73-130, 74-130, 75-130, 76-130, 77-130, 78-130, 79-130, 80-130, 81-130, 82-130, 83-130, 84-130, 85-130, 86-130, 87-130, 88-130, 89-130, 90-130, 91-130, 92-130, 93-130, 94-130, 95-130, 96-130, 97-130, 98-130, 99-130, 100-130, 101-130, 102-130, 103-130, 104-130, 105-130, 106-130, 107-130, 108-130, 109-130, 110-130, 111-130, 112-130, 113-130, 114-130, 115-130, 116-130, 117-130, 118-130, 119-130, 120-130, 121-130, 122-130, 123-130, 124-130, 125-130, 126-130, 21-129, 22-129, 23-129, 24-129, 25-129, 26-129, 27-129, 28-129, 29-129, 30-129, 31-129, 32-129, 33-129, 34-129, 35-129, 36-129, 37-129, 38-129, 39-129, 40-129, 41-129, 42-129, 43-129, 44-129, 45-129, 46-129, 47-129, 48-129, 49-129, 50-129, 51-129, 52-129, 53-129, 54-129, 55-129, 56-129, 57-129, 58-129, 59-129, 60-129, 61-129, 62-129, 63-129, 64-129, 65-129, 66-129, 67-129, 68-129, 69-129, 70-129, 71-129, 72-129, 73-129, 74-129, 75-129, 76-129, 77-129, 78-129, 79-129, 80-129, 81-129, 82-129, 83-129, 84-129, 85-129, 86-129, 87-129, 88-129, 89-129, 90-129, 91-129, 92-129, 93-129, 94-129, 95-129, 96-129, 97-129, 98-129, 99-129, 100-129, 101-129, 102-129, 103-129, 104-129, 105-129, 106-129, 107-129, 108-129, 109-129, 110-129, 111-129, 112-129, 113-129, 114-129, 115-129, 116-129, 117-129, 118-129, 119-129, 120-129, 121-129, 122-129, 123-129, 124-129, 125-129, 21-128, 22-128, 23-128, 24-128, 25-128, 26-128, 27-128, 28-128, 29-128, 30-128, 31-128, 32-128, 33-128, 34-128, 35-128, 36-128, 37-128, 38-128, 39-128, 40-128, 41-128, 42-128, 43-128, 44-128, 45-128, 46-128, 47-128, 48-128, 49-128, 50-128, 51-128, 52-128, 53-128, 54-128, 55-128, 56-128, 57-128, 58-128, 59-128, 60-128, 61-128, 62-128, 63-128, 64-128, 65-128, 66-128, 67-128, 68-128, 69-128, 70-128, 71-128, 72-128, 73-128, 74-128, 75-128, 76-128, 77-128, 78-128, 79-128, 80-128, 81-128, 82-128, 83-128, 84-128, 85-128, 86-128, 87-128, 88-128, 89-128, 90-128, 91-128, 92-128, 93-128, 94-128, 95-128, 96-128, 97-128, 98-128, 99-128, 100-128, 101-128, 102-128, 103-128, 104-128, 105-128, 106-128, 107-128, 108-128, 109-128, 110-128, 111-128, 112-128, 113-128, 114-128, 115-128, 116-128, 117-128, 118-128, 119-128, 120-128, 121-128, 122-128, 123-128, 124-128, 21-127, 22-127, 23-127, 24-127, 25-127, 26-127, 27-127, 28-127, 29-127, 30-127, 31-127, 32-127, 33-127, 34-127, 35-127, 36-127, 37-127, 38-127, 39-127, 40-127, 41-127, 42-127, 43-127, 44-127, 45-127, 46-127, 47-127, 48-127, 49-127, 50-127, 51-127, 52-127, 53-127, 54-127, 55-127, 56-127, 57-127, 58-127, 59-127, 60-127, 61-127, 62-127, 63-127, 64-127, 65-127, 66-127, 67-127, 68-127, 69-127, 70-127, 71-127, 72-127, 73-127, 74-127, 75-127, 76-127, 77-127, 78-127, 79-127, 80-127, 81-127, 82-127, 83-127, 84-127, 85-127, 86-127, 87-127, 88-127, 89-127, 90-127, 91-127, 92-127, 93-127, 94-127, 95-127, 96-127, 97-127, 98-127, 99-127, 100-127, 101-127, 102-127, 103-127, 104-127, 105-127, 106-127, 107-127, 108-127, 109-127, 110-127, 111-127, 112-127, 113-127, 114-127, 115-127, 116-127, 117-127, 118-127, 119-127, 120-127, 121-127, 122-127, 123-127, 21-126, 22-126, 23-126, 24-126, 25-126, 26-126, 27-126, 28-126, 29-126, 30-126, 31-126, 32-126, 33-126, 34-126, 35-126, 36-126, 37-126, 38-126, 39-126, 40-126, 41-126, 42-126, 43-126, 44-126, 45-126, 46-126, 47-126, 48-126, 49-126, 50-126, 51-126, 52-126, 53-126, 54-126, 55-126, 56-126, 57-126, 58-126, 59-126, 60-126, 61-126, 62-126, 63-126, 64-126, 65-126, 66-126, 67-126, 68-126, 69-126, 70-126, 71-126, 72-126, 73-126, 74-126, 75-126, 76-126, 77-126, 78-126, 79-126, 80-126, 81-126, 82-126, 83-126, 84-126, 85-126, 86-126, 87-126, 88-126, 89-126, 90-126, 91-126, 92-126, 93-126, 94-126, 95-126, 96-126, 97-126, 98-126, 99-126, 100-126, 101-126, 102-126, 103-126, 104-126, 105-126, 106-126, 107-126, 108-126, 109-126, 110-126, 111-126, 112-126, 113-126, 114-126, 115-126, 116-126, 117-126, 118-126, 119-126, 120-126, 121-126, 122-126, 21-125, 22-125, 23-125, 24-125, 25-125, 26-125, 27-125, 28-125, 29-125, 30-125, 31-125, 32-125, 33-125, 34-125, 35-125, 36-125, 37-125, 38-125, 39-125, 40-125, 41-125, 42-125, 43-125, 44-125, 45-125, 46-125, 47-125, 48-125, 49-125, 50-125, 51-125, 52-125, 53-125, 54-125, 55-125, 56-125, 57-125, 58-125, 59-125, 60-125, 61-125, 62-125, 63-125, 64-125, 65-125, 66-125, 67-125, 68-125, 69-125, 70-125, 71-125, 72-125, 73-125, 74-125, 75-125, 76-125, 77-125, 78-125, 79-125, 80-125, 81-125, 82-125, 83-125, 84-125, 85-125, 86-125, 87-125, 88-125, 89-125, 90-125, 91-125, 92-125, 93-125, 94-125, 95-125, 96-125, 97-125, 98-125, 99-125, 100-125, 101-125, 102-125, 103-125, 104-125, 105-125, 106-125, 107-125, 108-125, 109-125, 110-125, 111-125, 112-125, 113-125, 114-125, 115-125, 116-125, 117-125, 118-125, 119-125, 120-125, 121-125, 21-124, 22-124, 23-124, 24-124, 25-124, 26-124, 27-124, 28-124, 29-124, 30-124, 31-124, 32-124, 33-124, 34-124, 35-124, 36-124, 37-124, 38-124, 39-124, 40-124, 41-124, 42-124, 43-124, 44-124, 45-124, 46-124, 47-124, 48-124, 49-124, 50-124, 51-124, 52-124, 53-124, 54-124, 55-124, 56-124, 57-124, 58-124, 59-124, 60-124, 61-124, 62-124, 63-124, 64-124, 65-124, 66-124, 67-124, 68-124, 69-124, 70-124, 71-124, 72-124, 73-124, 74-124, 75-124, 76-124, 77-124, 78-124, 79-124, 80-124, 81-124, 82-124, 83-124, 84-124, 85-124, 86-124, 87-124, 88-124, 89-124, 90-124, 91-124, 92-124, 93-124, 94-124, 95-124, 96-124, 97-124, 98-124, 99-124, 100-124, 101-124, 102-124, 103-124, 104-124, 105-124, 106-124, 107-124, 108-124, 109-124, 110-124, 111-124, 112-124, 113-124, 114-124, 115-124, 116-124, 117-124, 118-124, 119-124, 120-124, 21-123, 22-123, 23-123, 24-123, 25-123, 26-123, 27-123, 28-123, 29-123, 30-123, 31-123, 32-123, 33-123, 34-123, 35-123, 36-123, 37-123, 38-123, 39-123, 40-123, 41-123, 42-123, 43-123, 44-123, 45-123, 46-123, 47-123, 48-123, 49-123, 50-123, 51-123, 52-123, 53-123, 54-123, 55-123, 56-123, 57-123, 58-123, 59-123, 60-123, 61-123, 62-123, 63-123, 64-123, 65-123, 66-123, 67-123, 68-123, 69-123, 70-123, 71-123, 72-123, 73-123, 74-123, 75-123, 76-123, 77-123, 78-123, 79-123, 80-123, 81-123, 82-123, 83-123, 84-123, 85-123, 86-123, 87-123, 88-123, 89-123, 90-123, 91-123, 92-123, 93-123, 94-123, 95-123, 96-123, 97-123, 98-123, 99-123, 100-123, 101-123, 102-123, 103-123, 104-123, 105-123, 106-123, 107-123, 108-123, 109-123, 110-123, 111-123, 112-123, 113-123, 114-123, 115-123, 116-123, 117-123, 118-123, 119-123, 21-122, 22-122, 23-122, 24-122, 25-122, 26-122, 27-122, 28-122, 29-122, 30-122, 31-122, 32-122, 33-122, 34-122, 35-122, 36-122, 37-122, 38-122, 39-122, 40-122, 41-122, 42-122, 43-122, 44-122, 45-122, 46-122, 47-122, 48-122, 49-122, 50-122, 51-122, 52-122, 53-122, 54-122, 55-122, 56-122, 57-122, 58-122, 59-122, 60-122, 61-122, 62-122, 63-122, 64-122, 65-122, 66-122, 67-122, 68-122, 69-122, 70-122, 71-122, 72-122, 73-122, 74-122, 75-122, 76-122, 77-122, 78-122, 79-122, 80-122, 81-122, 82-122, 83-122, 84-122, 85-122, 86-122, 87-122, 88-122, 89-122, 90-122, 91-122, 92-122, 93-122, 94-122, 95-122, 96-122, 97-122, 98-122, 99-122, 100-122, 101-122, 102-122, 103-122, 104-122, 105-122, 106-122, 107-122, 108-122, 109-122, 110-122, 111-122, 112-122, 113-122, 114-122, 115-122, 116-122, 117-122, 118-122, 21-121, 22-121, 23-121, 24-121, 25-121, 26-121, 27-121, 28-121, 29-121, 30-121, 31-121, 32-121, 33-121, 34-121, 35-121, 36-121, 37-121, 38-121, 39-121, 40-121, 41-121, 42-121, 43-121, 44-121, 45-121, 46-121, 47-121, 48-121, 49-121, 50-121, 51-121, 52-121, 53-121, 54-121, 55-121, 56-121, 57-121, 58-121, 59-121, 60-121, 61-121, 62-121, 63-121, 64-121, 65-121, 66-121, 67-121, 68-121, 69-121, 70-121, 71-121, 72-121, 73-121, 74-121, 75-121, 76-121, 77-121, 78-121, 79-121, 80-121, 81-121, 82-121, 83-121, 84-121, 85-121, 86-121, 87-121, 88-121, 89-121, 90-121, 91-121, 92-121, 93-121, 94-121, 95-121, 96-121, 97-121, 98-121, 99-121, 100-121, 101-121, 102-121, 103-121, 104-121, 105-121, 106-121, 107-121, 108-121, 109-121, 110-121, 111-121, 112-121, 113-121, 114-121, 115-121, 116-121, 117-121, 21-120, 22-120, 23-120, 24-120, 25-120, 26-120, 27-120, 28-120, 29-120, 30-120, 31-120, 32-120, 33-120, 34-120, 35-120, 36-120, 37-120, 38-120, 39-120, 40-120, 41-120, 42-120, 43-120, 44-120, 45-120, 46-120, 47-120, 48-120, 49-120, 50-120, 51-120, 52-120, 53-120, 54-120, 55-120, 56-120, 57-120, 58-120, 59-120, 60-120, 61-120, 62-120, 63-120, 64-120, 65-120, 66-120, 67-120, 68-120, 69-120, 70-120, 71-120, 72-120, 73-120, 74-120, 75-120, 76-120, 77-120, 78-120, 79-120, 80-120, 81-120, 82-120, 83-120, 84-120, 85-120, 86-120, 87-120, 88-120, 89-120, 90-120, 91-120, 92-120, 93-120, 94-120, 95-120, 96-120, 97-120, 98-120, 99-120, 100-120, 101-120, 102-120, 103-120, 104-120, 105-120, 106-120, 107-120, 108-120, 109-120, 110-120, 111-120, 112-120, 113-120, 114-120, 115-120, 116-120, 21-119, 22-119, 23-119, 24-119, 25-119, 26-119, 27-119, 28-119, 29-119, 30-119, 31-119, 32-119, 33-119, 34-119, 35-119, 36-119, 37-119, 38-119, 39-119, 40-119, 41-119, 42-119, 43-119, 44-119, 45-119, 46-119, 47-119, 48-119, 49-119, 50-119, 51-119, 52-119, 53-119, 54-119, 55-119, 56-119, 57-119, 58-119, 59-119, 60-119, 61-119, 62-119, 63-119, 64-119, 65-119, 66-119, 67-119, 68-119, 69-119, 70-119, 71-119, 72-119, 73-119, 74-119, 75-119, 76-119, 77-119, 78-119, 79-119, 80-119, 81-119, 82-119, 83-119, 84-119, 85-119, 86-119, 87-119, 88-119, 89-119, 90-119, 91-119, 92-119, 93-119, 94-119, 95-119, 96-119, 97-119, 98-119, 99-119, 100-119, 101-119, 102-119, 103-119, 104-119, 105-119, 106-119, 107-119, 108-119, 109-119, 110-119, 111-119, 112-119, 113-119, 114-119, 115-119, 21-118, 22-118, 23-118, 24-118, 25-118, 26-118, 27-118, 28-118, 29-118, 30-118, 31-118, 32-118, 33-118, 34-118, 35-118, 36-118, 37-118, 38-118, 39-118, 40-118, 41-118, 42-118, 43-118, 44-118, 45-118, 46-118, 47-118, 48-118, 49-118, 50-118, 51-118, 52-118, 53-118, 54-118, 55-118, 56-118, 57-118, 58-118, 59-118, 60-118, 61-118, 62-118, 63-118, 64-118, 65-118, 66-118, 67-118, 68-118, 69-118, 70-118, 71-118, 72-118, 73-118, 74-118, 75-118, 76-118, 77-118, 78-118, 79-118, 80-118, 81-118, 82-118, 83-118, 84-118, 85-118, 86-118, 87-118, 88-118, 89-118, 90-118, 91-118, 92-118, 93-118, 94-118, 95-118, 96-118, 97-118, 98-118, 99-118, 100-118, 101-118, 102-118, 103-118, 104-118, 105-118, 106-118, 107-118, 108-118, 109-118, 110-118, 111-118, 112-118, 113-118, 114-118, 21-117, 22-117, 23-117, 24-117, 25-117, 26-117, 27-117, 28-117, 29-117, 30-117, 31-117, 32-117, 33-117, 34-117, 35-117, 36-117, 37-117, 38-117, 39-117, 40-117, 41-117, 42-117, 43-117, 44-117, 45-117, 46-117, 47-117, 48-117, 49-117, 50-117, 51-117, 52-117, 53-117, 54-117, 55-117, 56-117, 57-117, 58-117, 59-117, 60-117, 61-117, 62-117, 63-117, 64-117, 65-117, 66-117, 67-117, 68-117, 69-117, 70-117, 71-117, 72-117, 73-117, 74-117, 75-117, 76-117, 77-117, 78-117, 79-117, 80-117, 81-117, 82-117, 83-117, 84-117, 85-117, 86-117, 87-117, 88-117, 89-117, 90-117, 91-117, 92-117, 93-117, 94-117, 95-117, 96-117, 97-117, 98-117, 99-117, 100-117, 101-117, 102-117, 103-117, 104-117, 105-117, 106-117, 107-117, 108-117, 109-117, 110-117, 111-117, 112-117, 113-117, 21-116, 22-116, 23-116, 24-116, 25-116, 26-116, 27-116, 28-116, 29-116, 30-116, 31-116, 32-116, 33-116, 34-116, 35-116, 36-116, 37-116, 38-116, 39-116, 40-116, 41-116, 42-116, 43-116, 44-116, 45-116, 46-116, 47-116, 48-116, 49-116, 50-116, 51-116, 52-116, 53-116, 54-116, 55-116, 56-116, 57-116, 58-116, 59-116, 60-116, 61-116, 62-116, 63-116, 64-116, 65-116, 66-116, 67-116, 68-116, 69-116, 70-116, 71-116, 72-116, 73-116, 74-116, 75-116, 76-116, 77-116, 78-116, 79-116, 80-116, 81-116, 82-116, 83-116, 84-116, 85-116, 86-116, 87-116, 88-116, 89-116, 90-116, 91-116, 92-116, 93-116, 94-116, 95-116, 96-116, 97-116, 98-116, 99-116, 100-116, 101-116, 102-116, 103-116, 104-116, 105-116, 106-116, 107-116, 108-116, 109-116, 110-116, 111-116, 112-116, 21-115, 22-115, 23-115, 24-115, 25-115, 26-115, 27-115, 28-115, 29-115, 30-115, 31-115, 32-115, 33-115, 34-115, 35-115, 36-115, 37-115, 38-115, 39-115, 40-115, 41-115, 42-115, 43-115, 44-115, 45-115, 46-115, 47-115, 48-115, 49-115, 50-115, 51-115, 52-115, 53-115, 54-115, 55-115, 56-115, 57-115, 58-115, 59-115, 60-115, 61-115, 62-115, 63-115, 64-115, 65-115, 66-115, 67-115, 68-115, 69-115, 70-115, 71-115, 72-115, 73-115, 74-115, 75-115, 76-115, 77-115, 78-115, 79-115, 80-115, 81-115, 82-115, 83-115, 84-115, 85-115, 86-115, 87-115, 88-115, 89-115, 90-115, 91-115, 92-115, 93-115, 94-115, 95-115, 96-115, 97-115, 98-115, 99-115, 100-115, 101-115, 102-115, 103-115, 104-115, 105-115, 106-115, 107-115, 108-115, 109-115, 110-115, 111-115, 21-114, 22-114, 23-114, 24-114, 25-114, 26-114, 27-114, 28-114, 29-114, 30-114, 31-114, 32-114, 33-114, 34-114, 35-114, 36-114, 37-114, 38-114, 39-114, 40-114, 41-114, 42-114, 43-114, 44-114, 45-114, 46-114, 47-114, 48-114, 49-114, 50-114, 51-114, 52-114, 53-114, 54-114, 55-114, 56-114, 57-114, 58-114, 59-114, 60-114, 61-114, 62-114, 63-114, 64-114, 65-114, 66-114, 67-114, 68-114, 69-114, 70-114, 71-114, 72-114, 73-114, 74-114, 75-114, 76-114, 77-114, 78-114, 79-114, 80-114, 81-114, 82-114, 83-114, 84-114, 85-114, 86-114, 87-114, 88-114, 89-114, 90-114, 91-114, 92-114, 93-114, 94-114, 95-114, 96-114, 97-114, 98-114, 99-114, 100-114, 101-114, 102-114, 103-114, 104-114, 105-114, 106-114, 107-114, 108-114, 109-114, 110-114, 21-113, 22-113, 23-113, 24-113, 25-113, 26-113, 27-113, 28-113, 29-113, 30-113, 31-113, 32-113, 33-113, 34-113, 35-113, 36-113, 37-113, 38-113, 39-113, 40-113, 41-113, 42-113, 43-113, 44-113, 45-113, 46-113, 47-113, 48-113, 49-113, 50-113, 51-113, 52-113, 53-113, 54-113, 55-113, 56-113, 57-113, 58-113, 59-113, 60-113, 61-113, 62-113, 63-113, 64-113, 65-113, 66-113, 67-113, 68-113, 69-113, 70-113, 71-113, 72-113, 73-113, 74-113, 75-113, 76-113, 77-113, 78-113, 79-113, 80-113, 81-113, 82-113, 83-113, 84-113, 85-113, 86-113, 87-113, 88-113, 89-113, 90-113, 91-113, 92-113, 93-113, 94-113, 95-113, 96-113, 97-113, 98-113, 99-113, 100-113, 101-113, 102-113, 103-113, 104-113, 105-113, 106-113, 107-113, 108-113, 109-113, 21-112, 22-112, 23-112, 24-112, 25-112, 26-112, 27-112, 28-112, 29-112, 30-112, 31-112, 32-112, 33-112, 34-112, 35-112, 36-112, 37-112, 38-112, 39-112, 40-112, 41-112, 42-112, 43-112, 44-112, 45-112, 46-112, 47-112, 48-112, 49-112, 50-112, 51-112, 52-112, 53-112, 54-112, 55-112, 56-112, 57-112, 58-112, 59-112, 60-112, 61-112, 62-112, 63-112, 64-112, 65-112, 66-112, 67-112, 68-112, 69-112, 70-112, 71-112, 72-112, 73-112, 74-112, 75-112, 76-112, 77-112, 78-112, 79-112, 80-112, 81-112, 82-112, 83-112, 84-112, 85-112, 86-112, 87-112, 88-112, 89-112, 90-112, 91-112, 92-112, 93-112, 94-112, 95-112, 96-112, 97-112, 98-112, 99-112, 100-112, 101-112, 102-112, 103-112, 104-112, 105-112, 106-112, 107-112, 108-112, 21-111, 22-111, 23-111, 24-111, 25-111, 26-111, 27-111, 28-111, 29-111, 30-111, 31-111, 32-111, 33-111, 34-111, 35-111, 36-111, 37-111, 38-111, 39-111, 40-111, 41-111, 42-111, 43-111, 44-111, 45-111, 46-111, 47-111, 48-111, 49-111, 50-111, 51-111, 52-111, 53-111, 54-111, 55-111, 56-111, 57-111, 58-111, 59-111, 60-111, 61-111, 62-111, 63-111, 64-111, 65-111, 66-111, 67-111, 68-111, 69-111, 70-111, 71-111, 72-111, 73-111, 74-111, 75-111, 76-111, 77-111, 78-111, 79-111, 80-111, 81-111, 82-111, 83-111, 84-111, 85-111, 86-111, 87-111, 88-111, 89-111, 90-111, 91-111, 92-111, 93-111, 94-111, 95-111, 96-111, 97-111, 98-111, 99-111, 100-111, 101-111, 102-111, 103-111, 104-111, 105-111, 106-111, 107-111, 21-110, 22-110, 23-110, 24-110, 25-110, 26-110, 27-110, 28-110, 29-110, 30-110, 31-110, 32-110, 33-110, 34-110, 35-110, 36-110, 37-110, 38-110, 39-110, 40-110, 41-110, 42-110, 43-110, 44-110, 45-110, 46-110, 47-110, 48-110, 49-110, 50-110, 51-110, 52-110, 53-110, 54-110, 55-110, 56-110, 57-110, 58-110, 59-110, 60-110, 61-110, 62-110, 63-110, 64-110, 65-110, 66-110, 67-110, 68-110, 69-110, 70-110, 71-110, 72-110, 73-110, 74-110, 75-110, 76-110, 77-110, 78-110, 79-110, 80-110, 81-110, 82-110, 83-110, 84-110, 85-110, 86-110, 87-110, 88-110, 89-110, 90-110, 91-110, 92-110, 93-110, 94-110, 95-110, 96-110, 97-110, 98-110, 99-110, 100-110, 101-110, 102-110, 103-110, 104-110, 105-110, 106-110, 21-109, 22-109, 23-109, 24-109, 25-109, 26-109, 27-109, 28-109, 29-109, 30-109, 31-109, 32-109, 33-109, 34-109, 35-109, 36-109, 37-109, 38-109, 39-109, 40-109, 41-109, 42-109, 43-109, 44-109, 45-109, 46-109, 47-109, 48-109, 49-109, 50-109, 51-109, 52-109, 53-109, 54-109, 55-109, 56-109, 57-109, 58-109, 59-109, 60-109, 61-109, 62-109, 63-109, 64-109, 65-109, 66-109, 67-109, 68-109, 69-109, 70-109, 71-109, 72-109, 73-109, 74-109, 75-109, 76-109, 77-109, 78-109, 79-109, 80-109, 81-109, 82-109, 83-109, 84-109, 85-109, 86-109, 87-109, 88-109, 89-109, 90-109, 91-109, 92-109, 93-109, 94-109, 95-109, 96-109, 97-109, 98-109, 99-109, 100-109, 101-109, 102-109, 103-109, 104-109, 105-109, 21-108, 22-108, 23-108, 24-108, 25-108, 26-108, 27-108, 28-108, 29-108, 30-108, 31-108, 32-108, 33-108, 34-108, 35-108, 36-108, 37-108, 38-108, 39-108, 40-108, 41-108, 42-108, 43-108, 44-108, 45-108, 46-108, 47-108, 48-108, 49-108, 50-108, 51-108, 52-108, 53-108, 54-108, 55-108, 56-108, 57-108, 58-108, 59-108, 60-108, 61-108, 62-108, 63-108, 64-108, 65-108, 66-108, 67-108, 68-108, 69-108, 70-108, 71-108, 72-108, 73-108, 74-108, 75-108, 76-108, 77-108, 78-108, 79-108, 80-108, 81-108, 82-108, 83-108, 84-108, 85-108, 86-108, 87-108, 88-108, 89-108, 90-108, 91-108, 92-108, 93-108, 94-108, 95-108, 96-108, 97-108, 98-108, 99-108, 100-108, 101-108, 102-108, 103-108, 104-108, 21-107, 22-107, 23-107, 24-107, 25-107, 26-107, 27-107, 28-107, 29-107, 30-107, 31-107, 32-107, 33-107, 34-107, 35-107, 36-107, 37-107, 38-107, 39-107, 40-107, 41-107, 42-107, 43-107, 44-107, 45-107, 46-107, 47-107, 48-107, 49-107, 50-107, 51-107, 52-107, 53-107, 54-107, 55-107, 56-107, 57-107, 58-107, 59-107, 60-107, 61-107, 62-107, 63-107, 64-107, 65-107, 66-107, 67-107, 68-107, 69-107, 70-107, 71-107, 72-107, 73-107, 74-107, 75-107, 76-107, 77-107, 78-107, 79-107, 80-107, 81-107, 82-107, 83-107, 84-107, 85-107, 86-107, 87-107, 88-107, 89-107, 90-107, 91-107, 92-107, 93-107, 94-107, 95-107, 96-107, 97-107, 98-107, 99-107, 100-107, 101-107, 102-107, 103-107, 21-106, 22-106, 23-106, 24-106, 25-106, 26-106, 27-106, 28-106, 29-106, 30-106, 31-106, 32-106, 33-106, 34-106, 35-106, 36-106, 37-106, 38-106, 39-106, 40-106, 41-106, 42-106, 43-106, 44-106, 45-106, 46-106, 47-106, 48-106, 49-106, 50-106, 51-106, 52-106, 53-106, 54-106, 55-106, 56-106, 57-106, 58-106, 59-106, 60-106, 61-106, 62-106, 63-106, 64-106, 65-106, 66-106, 67-106, 68-106, 69-106, 70-106, 71-106, 72-106, 73-106, 74-106, 75-106, 76-106, 77-106, 78-106, 79-106, 80-106, 81-106, 82-106, 83-106, 84-106, 85-106, 86-106, 87-106, 88-106, 89-106, 90-106, 91-106, 92-106, 93-106, 94-106, 95-106, 96-106, 97-106, 98-106, 99-106, 100-106, 101-106, 102-106, 21-105, 22-105, 23-105, 24-105, 25-105, 26-105, 27-105, 28-105, 29-105, 30-105, 31-105, 32-105, 33-105, 34-105, 35-105, 36-105, 37-105, 38-105, 39-105, 40-105, 41-105, 42-105, 43-105, 44-105, 45-105, 46-105, 47-105, 48-105, 49-105, 50-105, 51-105, 52-105, 53-105, 54-105, 55-105, 56-105, 57-105, 58-105, 59-105, 60-105, 61-105, 62-105, 63-105, 64-105, 65-105, 66-105, 67-105, 68-105, 69-105, 70-105, 71-105, 72-105, 73-105, 74-105, 75-105, 76-105, 77-105, 78-105, 79-105, 80-105, 81-105, 82-105, 83-105, 84-105, 85-105, 86-105, 87-105, 88-105, 89-105, 90-105, 91-105, 92-105, 93-105, 94-105, 95-105, 96-105, 97-105, 98-105, 99-105, 100-105, 101-105, 21-104, 22-104, 23-104, 24-104, 25-104, 26-104, 27-104, 28-104, 29-104, 30-104, 31-104, 32-104, 33-104, 34-104, 35-104, 36-104, 37-104, 38-104, 39-104, 40-104, 41-104, 42-104, 43-104, 44-104, 45-104, 46-104, 47-104, 48-104, 49-104, 50-104, 51-104, 52-104, 53-104, 54-104, 55-104, 56-104, 57-104, 58-104, 59-104, 60-104, 61-104, 62-104, 63-104, 64-104, 65-104, 66-104, 67-104, 68-104, 69-104, 70-104, 71-104, 72-104, 73-104, 74-104, 75-104, 76-104, 77-104, 78-104, 79-104, 80-104, 81-104, 82-104, 83-104, 84-104, 85-104, 86-104, 87-104, 88-104, 89-104, 90-104, 91-104, 92-104, 93-104, 94-104, 95-104, 96-104, 97-104, 98-104, 99-104, 100-104, 21-103, 22-103, 23-103, 24-103, 25-103, 26-103, 27-103, 28-103, 29-103, 30-103, 31-103, 32-103, 33-103, 34-103, 35-103, 36-103, 37-103, 38-103, 39-103, 40-103, 41-103, 42-103, 43-103, 44-103, 45-103, 46-103, 47-103, 48-103, 49-103, 50-103, 51-103, 52-103, 53-103, 54-103, 55-103, 56-103, 57-103, 58-103, 59-103, 60-103, 61-103, 62-103, 63-103, 64-103, 65-103, 66-103, 67-103, 68-103, 69-103, 70-103, 71-103, 72-103, 73-103, 74-103, 75-103, 76-103, 77-103, 78-103, 79-103, 80-103, 81-103, 82-103, 83-103, 84-103, 85-103, 86-103, 87-103, 88-103, 89-103, 90-103, 91-103, 92-103, 93-103, 94-103, 95-103, 96-103, 97-103, 98-103, 99-103, 21-102, 22-102, 23-102, 24-102, 25-102, 26-102, 27-102, 28-102, 29-102, 30-102, 31-102, 32-102, 33-102, 34-102, 35-102, 36-102, 37-102, 38-102, 39-102, 40-102, 41-102, 42-102, 43-102, 44-102, 45-102, 46-102, 47-102, 48-102, 49-102, 50-102, 51-102, 52-102, 53-102, 54-102, 55-102, 56-102, 57-102, 58-102, 59-102, 60-102, 61-102, 62-102, 63-102, 64-102, 65-102, 66-102, 67-102, 68-102, 69-102, 70-102, 71-102, 72-102, 73-102, 74-102, 75-102, 76-102, 77-102, 78-102, 79-102, 80-102, 81-102, 82-102, 83-102, 84-102, 85-102, 86-102, 87-102, 88-102, 89-102, 90-102, 91-102, 92-102, 93-102, 94-102, 95-102, 96-102, 97-102, 98-102, 21-101, 22-101, 23-101, 24-101, 25-101, 26-101, 27-101, 28-101, 29-101, 30-101, 31-101, 32-101, 33-101, 34-101, 35-101, 36-101, 37-101, 38-101, 39-101, 40-101, 41-101, 42-101, 43-101, 44-101, 45-101, 46-101, 47-101, 48-101, 49-101, 50-101, 51-101, 52-101, 53-101, 54-101, 55-101, 56-101, 57-101, 58-101, 59-101, 60-101, 61-101, 62-101, 63-101, 64-101, 65-101, 66-101, 67-101, 68-101, 69-101, 70-101, 71-101, 72-101, 73-101, 74-101, 75-101, 76-101, 77-101, 78-101, 79-101, 80-101, 81-101, 82-101, 83-101, 84-101, 85-101, 86-101, 87-101, 88-101, 89-101, 90-101, 91-101, 92-101, 93-101, 94-101, 95-101, 96-101, 97-101, 21-100, 22-100, 23-100, 24-100, 25-100, 26-100, 27-100, 28-100, 29-100, 30-100, 31-100, 32-100, 33-100, 34-100, 35-100, 36-100, 37-100, 38-100, 39-100, 40-100, 41-100, 42-100, 43-100, 44-100, 45-100, 46-100, 47-100, 48-100, 49-100, 50-100, 51-100, 52-100, 53-100, 54-100, 55-100, 56-100, 57-100, 58-100, 59-100, 60-100, 61-100, 62-100, 63-100, 64-100, 65-100, 66-100, 67-100, 68-100, 69-100, 70-100, 71-100, 72-100, 73-100, 74-100, 75-100, 76-100, 77-100, 78-100, 79-100, 80-100, 81-100, 82-100, 83-100, 84-100, 85-100, 86-100, 87-100, 88-100, 89-100, 90-100, 91-100, 92-100, 93-100, 94-100, 95-100, 96-100, 21-99, 22-99, 23-99, 24-99, 25-99, 26-99, 27-99, 28-99, 29-99, 30-99, 31-99, 32-99, 33-99, 34-99, 35-99, 36-99, 37-99, 38-99, 39-99, 40-99, 41-99, 42-99, 43-99, 44-99, 45-99, 46-99, 47-99, 48-99, 49-99, 50-99, 51-99, 52-99, 53-99, 54-99, 55-99, 56-99, 57-99, 58-99, 59-99, 60-99, 61-99, 62-99, 63-99, 64-99, 65-99, 66-99, 67-99, 68-99, 69-99, 70-99, 71-99, 72-99, 73-99, 74-99, 75-99, 76-99, 77-99, 78-99, 79-99, 80-99, 81-99, 82-99, 83-99, 84-99, 85-99, 86-99, 87-99, 88-99, 89-99, 90-99, 91-99, 92-99, 93-99, 94-99, 95-99, 21-98, 22-98, 23-98, 24-98, 25-98, 26-98, 27-98, 28-98, 29-98, 30-98, 31-98, 32-98, 33-98, 34-98, 35-98, 36-98, 37-98, 38-98, 39-98, 40-98, 41-98, 42-98, 43-98, 44-98, 45-98, 46-98, 47-98, 48-98, 49-98, 50-98, 51-98, 52-98, 53-98, 54-98, 55-98, 56-98, 57-98, 58-98, 59-98, 60-98, 61-98, 62-98, 63-98, 64-98, 65-98, 66-98, 67-98, 68-98, 69-98, 70-98, 71-98, 72-98, 73-98, 74-98, 75-98, 76-98, 77-98, 78-98, 79-98, 80-98, 81-98, 82-98, 83-98, 84-98, 85-98, 86-98, 87-98, 88-98, 89-98, 90-98, 91-98, 92-98, 93-98, 94-98, 21-97, 22-97, 23-97, 24-97, 25-97, 26-97, 27-97, 28-97, 29-97, 30-97, 31-97, 32-97, 33-97, 34-97, 35-97, 36-97, 37-97, 38-97, 39-97, 40-97, 41-97, 42-97, 43-97, 44-97, 45-97, 46-97, 47-97, 48-97, 49-97, 50-97, 51-97, 52-97, 53-97, 54-97, 55-97, 56-97, 57-97, 58-97, 59-97, 60-97, 61-97, 62-97, 63-97, 64-97, 65-97, 66-97, 67-97, 68-97, 69-97, 70-97, 71-97, 72-97, 73-97, 74-97, 75-97, 76-97, 77-97, 78-97, 79-97, 80-97, 81-97, 82-97, 83-97, 84-97, 85-97, 86-97, 87-97, 88-97, 89-97, 90-97, 91-97, 92-97, 93-97, 21-96, 22-96, 23-96, 24-96, 25-96, 26-96, 27-96, 28-96, 29-96, 30-96, 31-96, 32-96, 33-96, 34-96, 35-96, 36-96, 37-96, 38-96, 39-96, 40-96, 41-96, 42-96, 43-96, 44-96, 45-96, 46-96, 47-96, 48-96, 49-96, 50-96, 51-96, 52-96, 53-96, 54-96, 55-96, 56-96, 57-96, 58-96, 59-96, 60-96, 61-96, 62-96, 63-96, 64-96, 65-96, 66-96, 67-96, 68-96, 69-96, 70-96, 71-96, 72-96, 73-96, 74-96, 75-96, 76-96, 77-96, 78-96, 79-96, 80-96, 81-96, 82-96, 83-96, 84-96, 85-96, 86-96, 87-96, 88-96, 89-96, 90-96, 91-96, 92-96, 21-95, 22-95, 23-95, 24-95, 25-95, 26-95, 27-95, 28-95, 29-95, 30-95, 31-95, 32-95, 33-95, 34-95, 35-95, 36-95, 37-95, 38-95, 39-95, 40-95, 41-95, 42-95, 43-95, 44-95, 45-95, 46-95, 47-95, 48-95, 49-95, 50-95, 51-95, 52-95, 53-95, 54-95, 55-95, 56-95, 57-95, 58-95, 59-95, 60-95, 61-95, 62-95, 63-95, 64-95, 65-95, 66-95, 67-95, 68-95, 69-95, 70-95, 71-95, 72-95, 73-95, 74-95, 75-95, 76-95, 77-95, 78-95, 79-95, 80-95, 81-95, 82-95, 83-95, 84-95, 85-95, 86-95, 87-95, 88-95, 89-95, 90-95, 91-95, 21-94, 22-94, 23-94, 24-94, 25-94, 26-94, 27-94, 28-94, 29-94, 30-94, 31-94, 32-94, 33-94, 34-94, 35-94, 36-94, 37-94, 38-94, 39-94, 40-94, 41-94, 42-94, 43-94, 44-94, 45-94, 46-94, 47-94, 48-94, 49-94, 50-94, 51-94, 52-94, 53-94, 54-94, 55-94, 56-94, 57-94, 58-94, 59-94, 60-94, 61-94, 62-94, 63-94, 64-94, 65-94, 66-94, 67-94, 68-94, 69-94, 70-94, 71-94, 72-94, 73-94, 74-94, 75-94, 76-94, 77-94, 78-94, 79-94, 80-94, 81-94, 82-94, 83-94, 84-94, 85-94, 86-94, 87-94, 88-94, 89-94, 90-94, 21-93, 22-93, 23-93, 24-93, 25-93, 26-93, 27-93, 28-93, 29-93, 30-93, 31-93, 32-93, 33-93, 34-93, 35-93, 36-93, 37-93, 38-93, 39-93, 40-93, 41-93, 42-93, 43-93, 44-93, 45-93, 46-93, 47-93, 48-93, 49-93, 50-93, 51-93, 52-93, 53-93, 54-93, 55-93, 56-93, 57-93, 58-93, 59-93, 60-93, 61-93, 62-93, 63-93, 64-93, 65-93, 66-93, 67-93, 68-93, 69-93, 70-93, 71-93, 72-93, 73-93, 74-93, 75-93, 76-93, 77-93, 78-93, 79-93, 80-93, 81-93, 82-93, 83-93, 84-93, 85-93, 86-93, 87-93, 88-93, 89-93, 21-92, 22-92, 23-92, 24-92, 25-92, 26-92, 27-92, 28-92, 29-92, 30-92, 31-92, 32-92, 33-92, 34-92, 35-92, 36-92, 37-92, 38-92, 39-92, 40-92, 41-92, 42-92, 43-92, 44-92, 45-92, 46-92, 47-92, 48-92, 49-92, 50-92, 51-92, 52-92, 53-92, 54-92, 55-92, 56-92, 57-92, 58-92, 59-92, 60-92, 61-92, 62-92, 63-92, 64-92, 65-92, 66-92, 67-92, 68-92, 69-92, 70-92, 71-92, 72-92, 73-92, 74-92, 75-92, 76-92, 77-92, 78-92, 79-92, 80-92, 81-92, 82-92, 83-92, 84-92, 85-92, 86-92, 87-92, 88-92, 89-92, 90-92, 91-92, 92-92, 93-92, 94-92, 95-92, 96-92, 97-92, 98-92, 21-91, 22-91, 23-91, 24-91, 25-91, 26-91, 27-91, 28-91, 29-91, 30-91, 31-91, 32-91, 33-91, 34-91, 35-91, 36-91, 37-91, 38-91, 39-91, 40-91, 41-91, 42-91, 43-91, 44-91, 45-91, 46-91, 47-91, 48-91, 49-91, 50-91, 51-91, 52-91, 53-91, 54-91, 55-91, 56-91, 57-91, 58-91, 59-91, 60-91, 61-91, 62-91, 63-91, 64-91, 65-91, 66-91, 67-91, 68-91, 69-91, 70-91, 71-91, 72-91, 73-91, 74-91, 75-91, 76-91, 77-91, 78-91, 79-91, 80-91, 81-91, 82-91, 83-91, 84-91, 85-91, 86-91, 87-91, 21-90, 22-90, 23-90, 24-90, 25-90, 26-90, 27-90, 28-90, 29-90, 30-90, 31-90, 32-90, 33-90, 34-90, 35-90, 36-90, 37-90, 38-90, 39-90, 40-90, 41-90, 42-90, 43-90, 44-90, 45-90, 46-90, 47-90, 48-90, 49-90, 50-90, 51-90, 52-90, 53-90, 54-90, 55-90, 56-90, 57-90, 58-90, 59-90, 60-90, 61-90, 62-90, 63-90, 64-90, 65-90, 66-90, 67-90, 68-90, 69-90, 70-90, 71-90, 72-90, 73-90, 74-90, 75-90, 76-90, 77-90, 78-90, 79-90, 80-90, 81-90, 82-90, 83-90, 84-90, 85-90, 86-90, 21-89, 22-89, 23-89, 24-89, 25-89, 26-89, 27-89, 28-89, 29-89, 30-89, 31-89, 32-89, 33-89, 34-89, 35-89, 36-89, 37-89, 38-89, 39-89, 40-89, 41-89, 42-89, 43-89, 44-89, 45-89, 46-89, 47-89, 48-89, 49-89, 50-89, 51-89, 52-89, 53-89, 54-89, 55-89, 56-89, 57-89, 58-89, 59-89, 60-89, 61-89, 62-89, 63-89, 64-89, 65-89, 66-89, 67-89, 68-89, 69-89, 70-89, 71-89, 72-89, 73-89, 74-89, 75-89, 76-89, 77-89, 78-89, 79-89, 80-89, 81-89, 82-89, 83-89, 84-89, 85-89, 21-88, 22-88, 23-88, 24-88, 25-88, 26-88, 27-88, 28-88, 29-88, 30-88, 31-88, 32-88, 33-88, 34-88, 35-88, 36-88, 37-88, 38-88, 39-88, 40-88, 41-88, 42-88, 43-88, 44-88, 45-88, 46-88, 47-88, 48-88, 49-88, 50-88, 51-88, 52-88, 53-88, 54-88, 55-88, 56-88, 57-88, 58-88, 59-88, 60-88, 61-88, 62-88, 63-88, 64-88, 65-88, 66-88, 67-88, 68-88, 69-88, 70-88, 71-88, 72-88, 73-88, 74-88, 75-88, 76-88, 77-88, 78-88, 79-88, 80-88, 81-88, 82-88, 83-88, 84-88, 21-87, 22-87, 23-87, 24-87, 25-87, 26-87, 27-87, 28-87, 29-87, 30-87, 31-87, 32-87, 33-87, 34-87, 35-87, 36-87, 37-87, 38-87, 39-87, 40-87, 41-87, 42-87, 43-87, 44-87, 45-87, 46-87, 47-87, 48-87, 49-87, 50-87, 51-87, 52-87, 53-87, 54-87, 55-87, 56-87, 57-87, 58-87, 59-87, 60-87, 61-87, 62-87, 63-87, 64-87, 65-87, 66-87, 67-87, 68-87, 69-87, 70-87, 71-87, 72-87, 73-87, 74-87, 75-87, 76-87, 77-87, 78-87, 79-87, 80-87, 81-87, 82-87, 83-87, 21-86, 22-86, 23-86, 24-86, 25-86, 26-86, 27-86, 28-86, 29-86, 30-86, 31-86, 32-86, 33-86, 34-86, 35-86, 36-86, 37-86, 38-86, 39-86, 40-86, 41-86, 42-86, 43-86, 44-86, 45-86, 46-86, 47-86, 48-86, 49-86, 50-86, 51-86, 52-86, 53-86, 54-86, 55-86, 56-86, 57-86, 58-86, 59-86, 60-86, 61-86, 62-86, 63-86, 64-86, 65-86, 66-86, 67-86, 68-86, 69-86, 70-86, 71-86, 72-86, 73-86, 74-86, 75-86, 76-86, 77-86, 78-86, 79-86, 80-86, 81-86, 82-86, 21-85, 22-85, 23-85, 24-85, 25-85, 26-85, 27-85, 28-85, 29-85, 30-85, 31-85, 32-85, 33-85, 34-85, 35-85, 36-85, 37-85, 38-85, 39-85, 40-85, 41-85, 42-85, 43-85, 44-85, 45-85, 46-85, 47-85, 48-85, 49-85, 50-85, 51-85, 52-85, 53-85, 54-85, 55-85, 56-85, 57-85, 58-85, 59-85, 60-85, 61-85, 62-85, 63-85, 64-85, 65-85, 66-85, 67-85, 68-85, 69-85, 70-85, 71-85, 72-85, 73-85, 74-85, 75-85, 76-85, 77-85, 78-85, 79-85, 80-85, 81-85, 21-84, 22-84, 23-84, 24-84, 25-84, 26-84, 27-84, 28-84, 29-84, 30-84, 31-84, 32-84, 33-84, 34-84, 35-84, 36-84, 37-84, 38-84, 39-84, 40-84, 41-84, 42-84, 43-84, 44-84, 45-84, 46-84, 47-84, 48-84, 49-84, 50-84, 51-84, 52-84, 53-84, 54-84, 55-84, 56-84, 57-84, 58-84, 59-84, 60-84, 61-84, 62-84, 63-84, 64-84, 65-84, 66-84, 67-84, 68-84, 69-84, 70-84, 71-84, 72-84, 73-84, 74-84, 75-84, 76-84, 77-84, 78-84, 79-84, 80-84, 21-83, 22-83, 23-83, 24-83, 25-83, 26-83, 27-83, 28-83, 29-83, 30-83, 31-83, 32-83, 33-83, 34-83, 35-83, 36-83, 37-83, 38-83, 39-83, 40-83, 41-83, 42-83, 43-83, 44-83, 45-83, 46-83, 47-83, 48-83, 49-83, 50-83, 51-83, 52-83, 53-83, 54-83, 55-83, 56-83, 57-83, 58-83, 59-83, 60-83, 61-83, 62-83, 63-83, 64-83, 65-83, 66-83, 67-83, 68-83, 69-83, 70-83, 71-83, 72-83, 73-83, 74-83, 75-83, 76-83, 77-83, 78-83, 79-83, 21-82, 22-82, 23-82, 24-82, 25-82, 26-82, 27-82, 28-82, 29-82, 30-82, 31-82, 32-82, 33-82, 34-82, 35-82, 36-82, 37-82, 38-82, 39-82, 40-82, 41-82, 42-82, 43-82, 44-82, 45-82, 46-82, 47-82, 48-82, 49-82, 50-82, 51-82, 52-82, 53-82, 54-82, 55-82, 56-82, 57-82, 58-82, 59-82, 60-82, 61-82, 62-82, 63-82, 64-82, 65-82, 66-82, 67-82, 68-82, 69-82, 70-82, 71-82, 72-82, 73-82, 74-82, 75-82, 76-82, 77-82, 78-82, 21-81, 22-81, 23-81, 24-81, 25-81, 26-81, 27-81, 28-81, 29-81, 30-81, 31-81, 32-81, 33-81, 34-81, 35-81, 36-81, 37-81, 38-81, 39-81, 40-81, 41-81, 42-81, 43-81, 44-81, 45-81, 46-81, 47-81, 48-81, 49-81, 50-81, 51-81, 52-81, 53-81, 54-81, 55-81, 56-81, 57-81, 58-81, 59-81, 60-81, 61-81, 62-81, 63-81, 64-81, 65-81, 66-81, 67-81, 68-81, 69-81, 70-81, 71-81, 72-81, 73-81, 74-81, 75-81, 76-81, 77-81, 21-80, 22-80, 23-80, 24-80, 25-80, 26-80, 27-80, 28-80, 29-80, 30-80, 31-80, 32-80, 33-80, 34-80, 35-80, 36-80, 37-80, 38-80, 39-80, 40-80, 41-80, 42-80, 43-80, 44-80, 45-80, 46-80, 47-80, 48-80, 49-80, 50-80, 51-80, 52-80, 53-80, 54-80, 55-80, 56-80, 57-80, 58-80, 59-80, 60-80, 61-80, 62-80, 63-80, 64-80, 65-80, 66-80, 67-80, 68-80, 69-80, 70-80, 71-80, 72-80, 73-80, 74-80, 75-80, 76-80, 21-79, 22-79, 23-79, 24-79, 25-79, 26-79, 27-79, 28-79, 29-79, 30-79, 31-79, 32-79, 33-79, 34-79, 35-79, 36-79, 37-79, 38-79, 39-79, 40-79, 41-79, 42-79, 43-79, 44-79, 45-79, 46-79, 47-79, 48-79, 49-79, 50-79, 51-79, 52-79, 53-79, 54-79, 55-79, 56-79, 57-79, 58-79, 59-79, 60-79, 61-79, 62-79, 63-79, 64-79, 65-79, 66-79, 67-79, 68-79, 69-79, 70-79, 71-79, 72-79, 73-79, 74-79, 75-79, 21-78, 22-78, 23-78, 24-78, 25-78, 26-78, 27-78, 28-78, 29-78, 30-78, 31-78, 32-78, 33-78, 34-78, 35-78, 36-78, 37-78, 38-78, 39-78, 40-78, 41-78, 42-78, 43-78, 44-78, 45-78, 46-78, 47-78, 48-78, 49-78, 50-78, 51-78, 52-78, 53-78, 54-78, 55-78, 56-78, 57-78, 58-78, 59-78, 60-78, 61-78, 62-78, 63-78, 64-78, 65-78, 66-78, 67-78, 68-78, 69-78, 70-78, 71-78, 72-78, 73-78, 74-78, 21-77, 22-77, 23-77, 24-77, 25-77, 26-77, 27-77, 28-77, 29-77, 30-77, 31-77, 32-77, 33-77, 34-77, 35-77, 36-77, 37-77, 38-77, 39-77, 40-77, 41-77, 42-77, 43-77, 44-77, 45-77, 46-77, 47-77, 48-77, 49-77, 50-77, 51-77, 52-77, 53-77, 54-77, 55-77, 56-77, 57-77, 58-77, 59-77, 60-77, 61-77, 62-77, 63-77, 64-77, 65-77, 66-77, 67-77, 68-77, 69-77, 70-77, 71-77, 72-77, 73-77, 21-76, 22-76, 23-76, 24-76, 25-76, 26-76, 27-76, 28-76, 29-76, 30-76, 31-76, 32-76, 33-76, 34-76, 35-76, 36-76, 37-76, 38-76, 39-76, 40-76, 41-76, 42-76, 43-76, 44-76, 45-76, 46-76, 47-76, 48-76, 49-76, 50-76, 51-76, 52-76, 53-76, 54-76, 55-76, 56-76, 57-76, 58-76, 59-76, 60-76, 61-76, 62-76, 63-76, 64-76, 65-76, 66-76, 67-76, 68-76, 69-76, 70-76, 71-76, 72-76, 21-75, 22-75, 23-75, 24-75, 25-75, 26-75, 27-75, 28-75, 29-75, 30-75, 31-75, 32-75, 33-75, 34-75, 35-75, 36-75, 37-75, 38-75, 39-75, 40-75, 41-75, 42-75, 43-75, 44-75, 45-75, 46-75, 47-75, 48-75, 49-75, 50-75, 51-75, 52-75, 53-75, 54-75, 55-75, 56-75, 57-75, 58-75, 59-75, 60-75, 61-75, 62-75, 63-75, 64-75, 65-75, 66-75, 67-75, 68-75, 69-75, 70-75, 71-75, 21-74, 22-74, 23-74, 24-74, 25-74, 26-74, 27-74, 28-74, 29-74, 30-74, 31-74, 32-74, 33-74, 34-74, 35-74, 36-74, 37-74, 38-74, 39-74, 40-74, 41-74, 42-74, 43-74, 44-74, 45-74, 46-74, 47-74, 48-74, 49-74, 50-74, 51-74, 52-74, 53-74, 54-74, 55-74, 56-74, 57-74, 58-74, 59-74, 60-74, 61-74, 62-74, 63-74, 64-74, 65-74, 66-74, 67-74, 68-74, 69-74, 70-74, 21-73, 22-73, 23-73, 24-73, 25-73, 26-73, 27-73, 28-73, 29-73, 30-73, 31-73, 32-73, 33-73, 34-73, 35-73, 36-73, 37-73, 38-73, 39-73, 40-73, 41-73, 42-73, 43-73, 44-73, 45-73, 46-73, 47-73, 48-73, 49-73, 50-73, 51-73, 52-73, 53-73, 54-73, 55-73, 56-73, 57-73, 58-73, 59-73, 60-73, 61-73, 62-73, 63-73, 64-73, 65-73, 66-73, 67-73, 68-73, 69-73, 21-72, 22-72, 23-72, 24-72, 25-72, 26-72, 27-72, 28-72, 29-72, 30-72, 31-72, 32-72, 33-72, 34-72, 35-72, 36-72, 37-72, 38-72, 39-72, 40-72, 41-72, 42-72, 43-72, 44-72, 45-72, 46-72, 47-72, 48-72, 49-72, 50-72, 51-72, 52-72, 53-72, 54-72, 55-72, 56-72, 57-72, 58-72, 59-72, 60-72, 61-72, 62-72, 63-72, 64-72, 65-72, 66-72, 67-72, 68-72, 21-71, 22-71, 23-71, 24-71, 25-71, 26-71, 27-71, 28-71, 29-71, 30-71, 31-71, 32-71, 33-71, 34-71, 35-71, 36-71, 37-71, 38-71, 39-71, 40-71, 41-71, 42-71, 43-71, 44-71, 45-71, 46-71, 47-71, 48-71, 49-71, 50-71, 51-71, 52-71, 53-71, 54-71, 55-71, 56-71, 57-71, 58-71, 59-71, 60-71, 61-71, 62-71, 63-71, 64-71, 65-71, 66-71, 67-71, 21-70, 22-70, 23-70, 24-70, 25-70, 26-70, 27-70, 28-70, 29-70, 30-70, 31-70, 32-70, 33-70, 34-70, 35-70, 36-70, 37-70, 38-70, 39-70, 40-70, 41-70, 42-70, 43-70, 44-70, 45-70, 46-70, 47-70, 48-70, 49-70, 50-70, 51-70, 52-70, 53-70, 54-70, 55-70, 56-70, 57-70, 58-70, 59-70, 60-70, 61-70, 62-70, 63-70, 64-70, 65-70, 66-70, 21-69, 22-69, 23-69, 24-69, 25-69, 26-69, 27-69, 28-69, 29-69, 30-69, 31-69, 32-69, 33-69, 34-69, 35-69, 36-69, 37-69, 38-69, 39-69, 40-69, 41-69, 42-69, 43-69, 44-69, 45-69, 46-69, 47-69, 48-69, 49-69, 50-69, 51-69, 52-69, 53-69, 54-69, 55-69, 56-69, 57-69, 58-69, 59-69, 60-69, 61-69, 62-69, 63-69, 64-69, 65-69, 21-68, 22-68, 23-68, 24-68, 25-68, 26-68, 27-68, 28-68, 29-68, 30-68, 31-68, 32-68, 33-68, 34-68, 35-68, 36-68, 37-68, 38-68, 39-68, 40-68, 41-68, 42-68, 43-68, 44-68, 45-68, 46-68, 47-68, 48-68, 49-68, 50-68, 51-68, 52-68, 53-68, 54-68, 55-68, 56-68, 57-68, 58-68, 59-68, 60-68, 61-68, 62-68, 63-68, 64-68, 21-67, 22-67, 23-67, 24-67, 25-67, 26-67, 27-67, 28-67, 29-67, 30-67, 31-67, 32-67, 33-67, 34-67, 35-67, 36-67, 37-67, 38-67, 39-67, 40-67, 41-67, 42-67, 43-67, 44-67, 45-67, 46-67, 47-67, 48-67, 49-67, 50-67, 51-67, 52-67, 53-67, 54-67, 55-67, 56-67, 57-67, 58-67, 59-67, 60-67, 61-67, 62-67, 63-67, 21-66, 22-66, 23-66, 24-66, 25-66, 26-66, 27-66, 28-66, 29-66, 30-66, 31-66, 32-66, 33-66, 34-66, 35-66, 36-66, 37-66, 38-66, 39-66, 40-66, 41-66, 42-66, 43-66, 44-66, 45-66, 46-66, 47-66, 48-66, 49-66, 50-66, 51-66, 52-66, 53-66, 54-66, 55-66, 56-66, 57-66, 58-66, 59-66, 60-66, 61-66, 62-66, 21-65, 22-65, 23-65, 24-65, 25-65, 26-65, 27-65, 28-65, 29-65, 30-65, 31-65, 32-65, 33-65, 34-65, 35-65, 36-65, 37-65, 38-65, 39-65, 40-65, 41-65, 42-65, 43-65, 44-65, 45-65, 46-65, 47-65, 48-65, 49-65, 50-65, 51-65, 52-65, 53-65, 54-65, 55-65, 56-65, 57-65, 58-65, 59-65, 60-65, 61-65, 21-64, 22-64, 23-64, 24-64, 25-64, 26-64, 27-64, 28-64, 29-64, 30-64, 31-64, 32-64, 33-64, 34-64, 35-64, 36-64, 37-64, 38-64, 39-64, 40-64, 41-64, 42-64, 43-64, 44-64, 45-64, 46-64, 47-64, 48-64, 49-64, 50-64, 51-64, 52-64, 53-64, 54-64, 55-64, 56-64, 57-64, 58-64, 59-64, 60-64, 21-63, 22-63, 23-63, 24-63, 25-63, 26-63, 27-63, 28-63, 29-63, 30-63, 31-63, 32-63, 33-63, 34-63, 35-63, 36-63, 37-63, 38-63, 39-63, 40-63, 41-63, 42-63, 43-63, 44-63, 45-63, 46-63, 47-63, 48-63, 49-63, 50-63, 51-63, 52-63, 53-63, 54-63, 55-63, 56-63, 57-63, 58-63, 59-63, 21-62, 22-62, 23-62, 24-62, 25-62, 26-62, 27-62, 28-62, 29-62, 30-62, 31-62, 32-62, 33-62, 34-62, 35-62, 36-62, 37-62, 38-62, 39-62, 40-62, 41-62, 42-62, 43-62, 44-62, 45-62, 46-62, 47-62, 48-62, 49-62, 50-62, 51-62, 52-62, 53-62, 54-62, 55-62, 56-62, 57-62, 58-62, 21-61, 22-61, 23-61, 24-61, 25-61, 26-61, 27-61, 28-61, 29-61, 30-61, 31-61, 32-61, 33-61, 34-61, 35-61, 36-61, 37-61, 38-61, 39-61, 40-61, 41-61, 42-61, 43-61, 44-61, 45-61, 46-61, 47-61, 48-61, 49-61, 50-61, 51-61, 52-61, 53-61, 54-61, 55-61, 56-61, 57-61, 21-60, 22-60, 23-60, 24-60, 25-60, 26-60, 27-60, 28-60, 29-60, 30-60, 31-60, 32-60, 33-60, 34-60, 35-60, 36-60, 37-60, 38-60, 39-60, 40-60, 41-60, 42-60, 43-60, 44-60, 45-60, 46-60, 47-60, 48-60, 49-60, 50-60, 51-60, 52-60, 53-60, 54-60, 55-60, 56-60, 21-59, 22-59, 23-59, 24-59, 25-59, 26-59, 27-59, 28-59, 29-59, 30-59, 31-59, 32-59, 33-59, 34-59, 35-59, 36-59, 37-59, 38-59, 39-59, 40-59, 41-59, 42-59, 43-59, 44-59, 45-59, 46-59, 47-59, 48-59, 49-59, 50-59, 51-59, 52-59, 53-59, 54-59, 55-59, 21-58, 22-58, 23-58, 24-58, 25-58, 26-58, 27-58, 28-58, 29-58, 30-58, 31-58, 32-58, 33-58, 34-58, 35-58, 36-58, 37-58, 38-58, 39-58, 40-58, 41-58, 42-58, 43-58, 44-58, 45-58, 46-58, 47-58, 48-58, 49-58, 50-58, 51-58, 52-58, 53-58, 54-58, 21-57, 22-57, 23-57, 24-57, 25-57, 26-57, 27-57, 28-57, 29-57, 30-57, 31-57, 32-57, 33-57, 34-57, 35-57, 36-57, 37-57, 38-57, 39-57, 40-57, 41-57, 42-57, 43-57, 44-57, 45-57, 46-57, 47-57, 48-57, 49-57, 50-57, 51-57, 52-57, 53-57, 21-56, 22-56, 23-56, 24-56, 25-56, 26-56, 27-56, 28-56, 29-56, 30-56, 31-56, 32-56, 33-56, 34-56, 35-56, 36-56, 37-56, 38-56, 39-56, 40-56, 41-56, 42-56, 43-56, 44-56, 45-56, 46-56, 47-56, 48-56, 49-56, 50-56, 51-56, 52-56, 21-55, 22-55, 23-55, 24-55, 25-55, 26-55, 27-55, 28-55, 29-55, 30-55, 31-55, 32-55, 33-55, 34-55, 35-55, 36-55, 37-55, 38-55, 39-55, 40-55, 41-55, 42-55, 43-55, 44-55, 45-55, 46-55, 47-55, 48-55, 49-55, 50-55, 51-55, 21-54, 22-54, 23-54, 24-54, 25-54, 26-54, 27-54, 28-54, 29-54, 30-54, 31-54, 32-54, 33-54, 34-54, 35-54, 36-54, 37-54, 38-54, 39-54, 40-54, 41-54, 42-54, 43-54, 44-54, 45-54, 46-54, 47-54, 48-54, 49-54, 50-54, 21-53, 22-53, 23-53, 24-53, 25-53, 26-53, 27-53, 28-53, 29-53, 30-53, 31-53, 32-53, 33-53, 34-53, 35-53, 36-53, 37-53, 38-53, 39-53, 40-53, 41-53, 42-53, 43-53, 44-53, 45-53, 46-53, 47-53, 48-53, 49-53, 21-52, 22-52, 23-52, 24-52, 25-52, 26-52, 27-52, 28-52, 29-52, 30-52, 31-52, 32-52, 33-52, 34-52, 35-52, 36-52, 37-52, 38-52, 39-52, 40-52, 41-52, 42-52, 43-52, 44-52, 45-52, 46-52, 47-52, 48-52, 21-51, 22-51, 23-51, 24-51, 25-51, 26-51, 27-51, 28-51, 29-51, 30-51, 31-51, 32-51, 33-51, 34-51, 35-51, 36-51, 37-51, 38-51, 39-51, 40-51, 41-51, 42-51, 43-51, 44-51, 45-51, 46-51, 47-51, 21-50, 22-50, 23-50, 24-50, 25-50, 26-50, 27-50, 28-50, 29-50, 30-50, 31-50, 32-50, 33-50, 34-50, 35-50, 36-50, 37-50, 38-50, 39-50, 40-50, 41-50, 42-50, 43-50, 44-50, 45-50, 46-50, 21-49, 22-49, 23-49, 24-49, 25-49, 26-49, 27-49, 28-49, 29-49, 30-49, 31-49, 32-49, 33-49, 34-49, 35-49, 36-49, 37-49, 38-49, 39-49, 40-49, 41-49, 42-49, 43-49, 44-49, 45-49, 21-48, 22-48, 23-48, 24-48, 25-48, 26-48, 27-48, 28-48, 29-48, 30-48, 31-48, 32-48, 33-48, 34-48, 35-48, 36-48, 37-48, 38-48, 39-48, 40-48, 41-48, 42-48, 43-48, 44-48, 21-47, 22-47, 23-47, 24-47, 25-47, 26-47, 27-47, 28-47, 29-47, 30-47, 31-47, 32-47, 33-47, 34-47, 35-47, 36-47, 37-47, 38-47, 39-47, 40-47, 41-47, 42-47, 43-47, 21-46, 22-46, 23-46, 24-46, 25-46, 26-46, 27-46, 28-46, 29-46, 30-46, 31-46, 32-46, 33-46, 34-46, 35-46, 36-46, 37-46, 38-46, 39-46, 40-46, 41-46, 42-46, 21-45, 22-45, 23-45, 24-45, 25-45, 26-45, 27-45, 28-45, 29-45, 30-45, 31-45, 32-45, 33-45, 34-45, 35-45, 36-45, 37-45, 38-45, 39-45, 40-45, 41-45, 21-44, 22-44, 23-44, 24-44, 25-44, 26-44, 27-44, 28-44, 29-44, 30-44, 31-44, 32-44, 33-44, 34-44, 35-44, 36-44, 37-44, 38-44, 39-44, 40-44, 21-43, 22-43, 23-43, 24-43, 25-43, 26-43, 27-43, 28-43, 29-43, 30-43, 31-43, 32-43, 33-43, 34-43, 35-43, 36-43, 37-43, 38-43, 39-43, 21-42, 22-42, 23-42, 24-42, 25-42, 26-42, 27-42, 28-42, 29-42, 30-42, 31-42, 32-42, 33-42, 34-42, 35-42, 36-42, 37-42, 38-42, 21-41, 22-41, 23-41, 24-41, 25-41, 26-41, 27-41, 28-41, 29-41, 30-41, 31-41, 32-41, 33-41, 34-41, 35-41, 36-41, 37-41, 21-40, 22-40, 23-40, 24-40, 25-40, 26-40, 27-40, 28-40, 29-40, 30-40, 31-40, 32-40, 33-40, 34-40, 35-40, 36-40, 21-39, 22-39, 23-39, 24-39, 25-39, 26-39, 27-39, 28-39, 29-39, 30-39, 31-39, 32-39, 33-39, 34-39, 35-39, 21-38, 22-38, 23-38, 24-38, 25-38, 26-38, 27-38, 28-38, 29-38, 30-38, 31-38, 32-38, 33-38, 34-38, 21-37, 22-37, 23-37, 24-37, 25-37, 26-37, 27-37, 28-37, 29-37, 30-37, 31-37, 32-37, 33-37, 21-36, 22-36, 23-36, 24-36, 25-36, 26-36, 27-36, 28-36, 29-36, 30-36, 31-36, 32-36, 21-35, 22-35, 23-35, 24-35, 25-35, 26-35, 27-35, 28-35, 29-35, 30-35, 31-35, 21-34, 22-34, 23-34, 24-34, 25-34, 26-34, 27-34, 28-34, 29-34, 30-34, 21-33, 22-33, 23-33, 24-33, 25-33, 26-33, 27-33, 28-33, 29-33, 21-32, 22-32, 23-32, 24-32, 25-32, 26-32, 27-32, 28-32, 21-31, 22-31, 23-31, 24-31, 25-31, 26-31, 27-31, 21-30, 22-30, 23-30, 24-30, 25-30, 26-30, 21-29, 22-29, 23-29, 24-29, 25-29, 21-28, 22-28, 23-28, 24-28, 21-27, 22-27, 23-27, 21-26, 22-26, and 21-25.

(b) “NGAL analog” refers to a biologically active analog of NGAL, such as a biologically active analog of human NGAL. The analog can comprise truncations, deletions, insertions, substitutions, replacements, side-chain extensions, and fusions, such as a fusion with another protein, as well as combinations of any of the foregoing, which do not eliminate the biological activity of NGAL. Exemplary NGAL analogs include but are not limited to any mutated NGAL sequences set forth in any one or more of U.S. Provisional Application Nos. 60/981,470, 60/981,471 and 60/981,473, all filed on Oct. 19, 2007, and U.S. patent application Ser. Nos. 12/104,408, 12/104,410, and 12/104,413, all filed on Apr. 16, 2008, and each of which are incorporated by reference in their entireties for their teachings regarding same.

(c) “NGAL conjugate” refers to NGAL or a fragment thereof that includes at least one modifying moiety or at least one reactive entity attached thereto. Examples of modifying moieties include, but are not limited to, moieties that affect stability, solubility, and/or biological activity (e.g., hydrophilic polymers or oligomers, amphiphilic polymers or oligomers, and lipophilic polymers or oligomers), hydrophilic moieties, polyethylene glycol moieties, biocompatible water-soluble moieties, polycationic moieties, amphiphilic moieties, polyethylene glycol/alkyl-modifying moieties, etc. Such modifying moieties can be covalently bonded to NGAL or a fragment thereof. The modifying moiety can form a covalent bond with a component of blood, such as a blood protein, e.g., by way of an amino group, a hydroxyl group, or a thiol group present on the component. The amino group can form a covalent bond with reactive entities like carboxy, phosphoryl or acyl; the hydroxyl group can form a covalent bond with reactive entities like activated esters; and the thiol group can form a covalent bond with reactive entities like esters or mixed anhydrides. The preferred blood components are mobile blood components like serum albumin, immunoglobulins, or combinations thereof, and the preferred reactive entity comprises an anhydride like maleimide or a maleimide-containing group.

(d) “NGAL derivative” refers to an NGAL analog, an NGAL conjugate, or a recombinant form of NGAL.

(e) “Control” refers to a composition known to not contain an antibody to NGAL (“negative control”) or to contain an antibody to NGAL (“positive control”). A positive control can comprise a known concentration of an antibody to NGAL. “Control” and “positive control” may be used interchangeably herein to refer to a composition comprising a known concentration of an antibody to NGAL. A “positive control” can be used to establish assay performance characteristics and is a useful indicator of the integrity of reagents (e.g., analytes).

(f) “Series of calibrating compositions” refers to a plurality of compositions comprising a known concentration of an antibody to NGAL, wherein each of the compositions differs from the other compositions in the series by the concentration of the antibody to NGAL.

(g) In the context of immunoassays and kits described herein, “quality control reagents” include, but are not limited to, calibrators, controls, and sensitivity panels. A “calibrator” or “standard” typically is used (e.g., one or more, such as a plurality) in order to establish calibration (standard) curves for interpolation of the concentration of an analyte, such as an antibody or an analyte. Alternatively, a single calibrator, which is near a predetermined positive/negative cutoff, can be used. Multiple calibrators (i.e., more than one calibrator or a varying amount of calibrator(s)) can be used in conjunction so as to comprise a “sensitivity panel.”

(h) “Predetermined cutoff” and “predetermined level” refer generally to an assay cutoff value that is used to assess diagnostic/prognostic/therapeutic efficacy results by comparing the assay results against the predetermined cutoff/level, where the predetermined cutoff/level already has been linked or associated with various clinical parameters (e.g., severity of disease, progression/nonprogression/improvement, etc.). The present disclosure provides exemplary predetermined levels. However, it is well-known that cutoff values may vary depending on the nature of the immunoassay (e.g., antibodies employed, etc.). It further is well within the ordinary skill of one in the art to adapt the disclosure herein for other immunoassays to obtain immunoassay-specific cutoff values for those other immunoassays based on this disclosure. Whereas the precise value of the predetermined cutoff/level may vary between assays, the correlations as described herein should be generally applicable.

(i) “Sample,” “test sample,” and “patient sample” may be used interchangeably herein. The sample, such as a sample of urine, serum, or plasma, can be obtained from a subject or patient using routine techniques known in the art. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

(j) “Patient” and “subject” may be used interchangeably herein to refer to an animal, such as a bird (e.g., a duck or a goose), a shark, a whale, and a mammal, including a non-primate (for example, a cow, pig, camel, llama, horse, goat, rabbit, sheep, hamster, guinea pig, cat, dog, rat, and mouse) and a primate (for example, a monkey, a chimpanzee, and a human). Preferably, the patient or subject is a human, such as a human at risk for cardiovascular disease, a human having cardiovascular disease, a human at risk for renal disease, or a human having renal disease.

(k) “At least one component,” “component,” and “components” refer generally to a capture antibody, a detection or conjugate antibody, a calibrator, a control, a sensitivity panel, a container, a buffer, a diluent, a salt, an enzyme, a co-factor for an enzyme, a detection reagent, a pretreatment reagent/solution, a substrate (e.g., as a solution), a stop solution, and the like that can be included in a kit for assay of a test sample, such as a patient urine, serum or plasma sample, in accordance with the methods described herein and other methods known in the art. Some components can be in solution or lyophilized for reconstitution for use in an assay.

(l) “Antibody” (Ab) and “antibodies” (Abs) refer to monoclonal antibodies, multispecific antibodies, human antibodies, humanized antibodies (fully or partially humanized), animal antibodies (such as, but not limited to, a bird (for example, a duck or a goose), a shark, a whale, and a mammal, including a non-primate (for example, a cow, pig, camel, llama, horse, goat, rabbit, sheep, hamster, guinea pig, cat, dog, rat, mouse, etc.) or a non-human primate (for example, a monkey, a chimpanzee, etc.), recombinant antibodies, chimeric antibodies, single-chain Fvs (“scFv”), single chain antibodies, single domain antibodies, Fab fragments, F(ab′) fragments, F(ab′)₂ fragments, disulfide-linked Fvs (“sdFv”), and anti-idiotypic (“anti-Id”) antibodies, dual-domain antibodies (e.g., dual variable domain antibodies, or DVD-IgGs), and functionally active epitope-binding fragments of any of the above. In particular, antibodies include immunoglobulin molecules and immunologically active fragments of immunoglobulin molecules, namely, molecules that contain an analyte-binding site. Immunoglobulin molecules can be of any type (for example, IgG, IgE, IgM, IgD, IgA and IgY), class (for example, IgG₁, IgG₂, IgG₃, IgG₄, IgA₁ and IgA₂), or subclass. An antibody, whose affinity (namely, KD, kd or ka) has been increased or improved via the screening of a combinatory antibody library that has been prepared using bio-display, is referred to as an “affinity maturated antibody.” For simplicity sake, an antibody against an analyte is frequently referred to herein as being either an “anti-analyte antibody” or an “analyte antibody” (e.g., an anti-NGAL antibody or an NGAL antibody).

(m) “Autoantibody” and “autoantibodies” refer to endogenous antibodies that bind to (or “react with”) an analyte that occurs naturally in the animal in which the antibody is produced. In the context of the present disclosure, the analyte is NGAL or a fragment thereof. Use of “at least one” and “one or more” with respect to autoantibodies means at least one or one or more types or populations of autoantibodies, i.e., such as autoantibodies that react with different epitopes on NGAL or fragments thereof. For simplicity sake, an autoantibody against an analyte is frequently referred to herein as being either an “anti-analyte autoantibody” or an “analyte autoantibody” (e.g., an anti-NGAL autoantibody or an NGAL autoantibody).

(n) “Label” and “detectable label” mean a moiety attached to an antibody or an analyte to render the reaction between the antibody and the analyte detectable, and the antibody or analyte so labeled is referred to as “detectably labeled.” A label can produce a signal that is detectable by visual or instrumental means. Various labels include signal-producing substances, such as chromogens, fluorescent compounds, chemiluminescent compounds, radioactive compounds, and the like. Representative examples of labels include moieties that produce light, e.g., acridinium compounds, and moieties that produce fluorescence, e.g., fluorescein. Other labels are described herein.

(o) “CPSP acridinium-9-carboxamide” means 9-[[(3-Carboxypropyl)[(4-methylphenyl)sulfonyl]amino]-carbonyl]-10-(3-sulfopropyl)acridinium inner salt.

(p) “SPSP acridinium-9-carboxamide” means 9-[[[[4-(3-carboxypropyl)phenyl]sulfonyl] (3-sulfopropyl)amino]carbonyl]-10-(3-sulfopropyl)-acridinium inner salt.

(q) “Tracer” means an analyte or analyte fragment conjugated to a label, such as NGAL (or a fragment thereof) conjugated to a label, e.g., a fluorescein moiety, wherein the analyte conjugated to the label can effectively compete with the analyte for sites on an antibody specific for the analyte.

(r) A “solid phase” refers to any material that is insoluble, or can be made insoluble by a subsequent reaction. The solid phase can be chosen for its intrinsic ability to attract and immobilize a capture agent. Alternatively, the solid phase can have affixed thereto a linking agent that has the ability to attract and immobilize the capture agent. The linking agent can, for example, include a charged substance that is oppositely charged with respect to the capture agent itself or to a charged substance conjugated to the capture agent. In general, the linking agent can be any binding partner (preferably specific) that is immobilized on (attached to) the solid phase and that has the ability to immobilize the capture agent through a binding reaction. The linking agent enables the indirect binding of the capture agent to a solid phase material before the performance of the assay or during the performance of the assay. The solid phase can, for example, be plastic, derivatized plastic, magnetic or non-magnetic metal, glass or silicon, including, for example, a test tube, microtiter well, sheet, bead, microparticle, chip, and other configurations known to those of ordinary skill in the art.

(s) “Specific binding partner” is a member of a specific binding pair. A specific binding pair comprises two different molecules, which specifically bind to each other through chemical or physical means. Therefore, in addition to antigen and antibody specific binding pairs of common immunoassays, other specific binding pairs can include biotin and avidin (or streptavidin), carbohydrates and lectins, complementary nucleotide sequences, effector and receptor molecules, cofactors and enzymes, enzyme inhibitors, and enzymes and the like. Furthermore, specific binding pairs can include members that are analogs of the original specific binding members, for example, an analyte-analog. Immunoreactive specific binding members include antigens, antigen fragments, and antibodies, including monoclonal and polyclonal antibodies as well as complexes and fragments thereof, whether isolated or recombinantly produced.

(t) “Hydrogen peroxide-generating enzyme” refers to an enzyme that can generate hydrogen peroxide. Examples of hydrogen peroxide-generating enzymes are listed below in Table 1.

TABLE 1

| | IUBMB Enzyme | |
|---|---|---|
| Common Name | Nomenclature | Preferred Substrate |
| (R)-6-hydroxynicotine oxidase | EC 1.5.3.6 | (R)-6-hydroxynicotine |
| (S)-2-hydroxy acid oxidase | EC 1.1.3.15 | S)-2-hydroxy acid |
| (S)-6-hydroxynicotine oxidase | EC 1.5.3.5 | (S)-6-hydroxynicotine |
| 3-aci-nitropropanoate oxidase | EC 1.7.3.5 | 3-aci-nitropropanoate |
| 3-hydroxyanthranilate oxidase | EC 1.10.3.5 | 3-hydroxyanthranilate |
| 4-hydroxymandelate oxidase | EC 1.1.3.19 | (S)-2-hydroxy-2-(4- |
| | | hydroxyphenyl)acetate |
| 6-hydroxynicotinate dehydrogenase | EC 1.17.3.3 | 6-hydroxynicotinate |
| Abscisic-aldehyde oxidase | EC 1.2.3.14 | abscisic aldehyde |
| acyl-CoA oxidase | EC 1.3.3.6 | acyl-CoA |
| Alcohol oxidase | EC 1.1.3.13 | a primary alcohol |
| Aldehyde oxidase | EC 1.2.3.1 | an aldehyde |
| amine oxidase | | |
| amine oxidase (copper-containing) | EC 1.4.3.6 | primary monoamines, |
| | | diamines and histamine |
| amine oxidase (flavin-containing) | EC 1.4.3.4 | a primary amine |
| aryl-alcohol oxidase | EC 1.1.3.7 | an aromatic primary |
| | | alcohol (2-naphthyl)methanol |
| | | 3-methoxybenzyl alcohol |
| aryl-aldehyde oxidase | EC 1.2.3.9 | an aromatic aldehyde |
| Catechol oxidase | EC 1.1.3.14 | Catechol |
| Cholesterol oxidase | EC 1.1.3.6 | Cholesterol |
| Choline oxidase | EC 1.1.3.17 | Choline |
| columbamine oxidase | EC 1.21.3.2 | Columbamine |
| cyclohexylamine oxidase | EC 1.4.3.12 | Cyclohexylamine |
| cytochrome c oxidase | EC 1.9.3.1 | |
| D-amino-acid oxidase | EC 1.4.3.3 | a D-amino acid |
| D-arabinono-1,4-lactone oxidase | EC 1.1.3.37 | D-arabinono-1,4-lactone |
| D-arabinono-1,4-lactone oxidase | EC 1.1.3.37 | D-arabinono-1,4-lactone |
| D-aspartate oxidase | EC 1.4.3.1 | D-aspartate |
| D-glutamate oxidase | EC 1.4.3.7 | D-glutamate |
| D-glutamate(D-aspartate) oxidase | EC 1.4.3.15 | D-glutamate |
| dihydrobenzophenanthridine | EC 1.5.3.12 | dihydrosanguinarine |
| oxidase | | |
| dihydroorotate oxidase | EC 1.3.3.1 | (S)-dihydroorotate |
| dihydrouracil oxidase | EC 1.3.3.7 | 5,6-dihydrouracil |
| dimethylglycine oxidase | EC 1.5.3.10 | N,N-dimethylglycine |
| D-mannitol oxidase | EC 1.1.3.40 | Mannitol |
| Ecdysone oxidase | EC 1.1.3.16 | Ecdysone |
| ethanolamine oxidase | EC 1.4.3.8 | Ethanolamine |
| Galactose oxidase | EC 1.1.3.9 | D-galactose |
| Glucose oxidase | EC 1.1.3.4 | β-D-glucose |
| Glutathione oxidase | EC 1.8.3.3 | Glutathione |
| glycerol-3-phosphate oxidase | EC 1.1.3.21 | sn-glycerol 3-phosphate |
| Glycine oxidase | EC 1.4.3.19 | Glycine |
| glyoxylate oxidase | EC 1.2.3.5 | Glyoxylate |
| hexose oxidase | EC 1.1.3.5 | D-glucose, |
| | | D-galactose |
| | | D-mannose |
| | | maltose |
| | | lactose |
| | | cellobiose |
| hydroxyphytanate oxidase | EC 1.1.3.27 | L-2-hydroxyphytanate |
| indole-3-acetaldehyde oxidase | EC 1.2.3.7 | (indol-3-yl)acetaldehyde |
| lactic acid oxidase | | Lactic acid |
| L-amino-acid oxidase | EC 1.4.3.2 | an L-amino acid |
| L-aspartate oxidase | EC 1.4.3.16 | L-aspartate |
| L-galactonolactone oxidase | EC 1.3.3.12 | L-galactono-1,4-lactone |
| L-glutamate oxidase | EC 1.4.3.11 | L-glutamate |
| L-gulonolactone oxidase | EC 1.1.3.8 | L-gulono-1,4-lactone |
| L-lysine 6-oxidase | EC 1.4.3.20 | L-lysine |
| L-lysine oxidase | EC 1.4.3.14 | L-lysine |
| long-chain-alcohol oxidase | EC 1.1.3.20 | A long-chain-alcohol |
| L-pipecolate oxidase | EC 1.5.3.7 | L-pipecolate |
| L-sorbose oxidase | EC 1.1.3.11 | L-sorbose |
| malate oxidase | EC 1.1.3.3 | (S)-malate |
| methanethiol oxidase | EC 1.8.3.4 | Methanethiol |
| monoamino acid oxidase | | |
| N⁶-methyl-lysine oxidase | EC 1.5.3.4 | 6-N-methyl-L-lysine |
| N-acylhexosamine oxidase | EC 1.1.3.29 | N-acetyl-D-glucosamine |
| | | N-glycolylglucosamine |
| | | N-acetylgalactosamine |
| | | N-acetylmannosamine. |
| NAD(P)H oxidase | EC 1.6.3.1 | NAD(P)H |
| Nitroalkane oxidase | EC 1.7.3.1 | a nitroalkane |
| N-methyl-L-amino-acid oxidase | EC 1.5.3.2 | an N-methyl-L-amino |
| | | acid |
| nucleoside oxidase | EC 1.1.3.39 | Adenosine |
| Oxalate oxidase | EC 1.2.3.4 | Oxalate |
| polyamine oxidase | EC 1.5.3.11 | 1-N-acetylspermine |
| Polyphenol oxidase | EC 1.14.18.1 | |
| Polyvinyl-alcohol oxidase | EC 1.1.3.30 | polyvinyl alcohol |
| prenylcysteine oxidase | EC 1.8.3.5 | an S-prenyl-L-cysteine |
| Protein-lysine 6-oxidase | EC 1.4.3.13 | peptidyl-L-lysyl-peptide |
| putrescine oxidase | EC 1.4.3.10 | butane-1,4-diamine |
| Pyranose oxidase | EC 1.1.3.10 | D-glucose |
| | | D-xylose |
| | | L-sorbose |
| | | D-glucono-1,5-lactone |
| Pyridoxal 5′-phosphate synthase | EC 1.4.3.5 | pyridoxamine 5′- |
| | | phosphate |
| pyridoxine 4-oxidase | EC 1.1.3.12 | Pyridoxine |
| pyrroloquinoline-quinone synthase | EC 1.3.3.11 | 6-(2-amino-2- |
| | | carboxyethyl)-7,8-dioxo- |
| | | 1,2,3,4,5,6,7,8- |
| | | octahydroquinoline-2,4- |
| | | dicarboxylate |
| Pyruvate oxidase | EC 1.2.3.3 | Pyruvate |
| Pyruvate oxidase (CoA-acetylating) | EC 1.2.3.6 | Pyruvate |
| Reticuline oxidase | EC 1.21.3.3 | Reticuline |
| retinal oxidase | EC 1.2.3.11 | Retinal |
| Rifamycin-B oxidase | EC 1.10.3.6 | rifamycin-B |
| Sarcosine oxidase | EC 1.5.3.1 | Sarcosine |
| secondary-alcohol oxidase | EC 1.1.3.18 | a secondary alcohol |
| sulfite oxidase | EC 1.8.3.1 | Sulfite |
| superoxide dismutase | EC 1.15.1.1 | Superoxide |
| superoxide reductase | EC 1.15.1.2 | Superoxide |
| tetrahydroberberine oxidase | EC 1.3.3.8 | (S)-tetrahydroberberine |
| Thiamine oxidase | EC 1.1.3.23 | Thiamine |
| tryptophan α,β-oxidase | EC 1.3.3.10 | L-tryptophan |
| urate oxidase (uricase, uric acid | EC 1.7.3.3 | uric acid |
| oxidase) | | |
| Vanillyl-alcohol oxidase | EC 1.1.3.38 | vanillyl alcohol |
| Xanthine oxidase | EC 1.17.3.2 | Xanthine |
| xylitol oxidase | EC 1.1.3.41 | Xylitol |

(u) “Cardiovascular disease” refers to various clinical diseases, disorders or conditions involving the heart, blood vessels or circulation. The diseases, disorders or conditions can be due to atherosclerotic impairment of coronary, cerebral or peripheral arteries. Cardiovascular disease includes, but is not limited to, coronary artery disease, peripheral vascular disease, atherosclerosis, hypertension, myocardial infarction (i.e., heart attack, e.g., primary or secondary, which occurs when an area of heart muscle dies or is damaged because of an inadequate supply of oxygen to that area), myocarditis, acute coronary syndrome, angina pectoris (i.e., chest discomfort caused by inadequate blood flow through the blood vessels (coronary vessels) of the myocardium), sudden cardiac death, cerebral infarction, restenosis, syncope, ischemia, transient ischemic attack, reperfusion injury, vascular occlusion, carotid obstructive disease, cardiovascular autoimmune disease, etc. By “cardiovascular autoimmune disease” is meant any deviation from a healthy or normal condition of the heart that is due to an underlying autoimmune disease, including any structural or functional abnormality of the heart, or of the blood vessels supplying the heart, that impairs typical functioning. Examples of cardiovascular autoimmune diseases include myocarditis, cardiomyopathy, and ischemic heart disease, each due to an underlying autoimmune disease. “Myocarditis” refers to inflammation of the myocardium. Myocarditis can be caused by a variety of conditions, such as viral infection, sarcoidosis, rheumatic fever, autoimmune diseases (such as systemic lupus erythematosus, etc.), and pregnancy. “Cardiomyopathy” refers to a weakening of the heart muscle or a change in heart muscle structure. It is often associated with inadequate heart pumping or other heart function abnormalities. Cardiomyopathy can be caused by viral infections, heart attacks, alcoholism, long-term, severe high blood pressure, nutritional deficiencies (particularly selenium, thiamine, and L-camitine), systemic lupus erythematosus, celiac disease, and end-stage kidney disease. Types of cardiomyopathy include dilated cardiomyopathy, hypertrophic cardiomyopathy, and restrictive cardiomyopathy. “Dilated cardiomyopathy” refers to a global, usually idiopathic, myocardial disorder characterized by a marked enlargement and inadequate function of the left ventricle. Dilated cardiomyopathy includes ischemic cardiomyopathy, idiopathic cardiomyopathy, hypertensive cardiomyopathy, infectious cardiomyopathy, alcoholic cardiomyopathy, toxic cardiomyopathy, and peripartum cardiomyopathy. “Hypertrophic cardiomyopathy” refers to a condition resulting from the right and left heart muscles growing to be different sizes. “Restrictive cardiomyopathy” refers to a condition characterized by the heart muscle's inability to relax between contractions, which prevents it from filling sufficiently. “Ischemic heart disease” refers to any condition in which heart muscle is damaged or works inefficiently because of an absence or relative deficiency of its blood supply; most often caused by atherosclerosis, it includes angina pectoris, acute myocardial infarction, and chronic ischemic heart disease.

(v) “Heart failure” refers to a condition in which the heart cannot pump blood efficiently to the rest of the body. Heart failure can be due to damage to the heart or narrowing of the arteries due to infarction, cardiomyopathy (primary or secondary), hypertension, coronary artery disease, valve disease, birth defects or infection. Heart failure can further be described as chronic, congestive, acute, decompensated, systolic or diastolic. The New York Heart Association (NYHA) classification describes the severity of the disease based on functional capacity of the patient; NYHA class can progress and/or regress based on treatment or lack of response to treatment. In heart failure, “increased severity” of cardiovascular disease refers to the worsening of disease as indicated by increased NYHA classification, to, for example, Class III or Class IV, and “reduced severity” of cardiovascular disease refers to an improvement of the disease as indicated by reduced NYHA classification, from, for example, class III or IV to class II or I.

(w) “Autoimmune disease” refers to the loss of immunological tolerance to self antigens. Some criteria for a diagnosis of autoimmune disease include: (1) the presence of circulating autoantibodies; (2) autoantibodies observed in the affected organ; (3) target antigen identified; (4) inducible in an animal model either by immunization with antigen, serum, or autoantibody transfer; and (5) responsive to immunosuppressive therapy or immunoabsorption. Other characteristics of autoimmune disease include its: (a) increased prevalence in women; (b) familial clustering (although this varies with disease); (c) asymptomatic risk (i.e., the presence of autoantibodies may precede the disease by years); (d) periodic nature; and (e) chronic nature.

(x) “Autoimmunity” refers to one or more immune responses directed against host antigens, characterized, for example, by the presence of autoantibodies or T lymphocytes reactive with host antigens.

(y) “Renal disease” refers to any disease, disorder, or damage to or injury of the kidney, including, for example, acute renal failure, acute nephritic syndrome, analgesic nephropathy, atheroembolic renal disease, chronic renal failure, chronic nephritis, congenital nephritic syndrome, end-stage renal disease, Goodpasture syndrome, interstitial nephritis, renal cancer, renal damange, renal infection, renal injury, kidney stones, lupus nephritis, membranoproliferative GN I, membranoproliferative GN II, membranous nephropathy, minimal change disease, necrotizing glomerulonephritis, nephroblastoma, nephrocalcinosis, nephrogenic diabetes insipidus, nephropathy—IgA, nephrosis (nephrotic syndrome), polycystic kidney disease, post-streptococcal GN, reflux nephropathy, renal artery embolism, renal artery stenosis, renal papillary necrosis, renal tubular acidosis type I, renal tubular acidosis type II, renal underperfusion, renal vein thrombosis, and the like.

(z) “Risk” refers to the possibility or probability of a particular event occurring either presently, or, at some point in the future. “Risk stratification” refers to an array of known clinical risk factors that allows physicians to classify patients into a low, moderate, high or highest risk of developing a particular disease, disorder or condition.

(aa) “About” refers to approximately a +/−10% variation from the stated value. It is to be understood that such a variation is always included in any given value provided herein, whether or not specific reference is made to it.

(bb) As used herein, the term “reliability” means that with respect to a given result or value (such as that obtained from an assay, such as an immunoassay or a chemiluminescent assay) that there is at least about a 90% certainty (e.g., from about a 90% certainty to about a 100% certainty) that said result or value is accurate or correct, preferably at least about a 95% certainty (e.g., from about a 95% certainty to about a 100% certainty) that said result or value is accurate or correct.

The above terminology is provided for the purpose of describing particular embodiments. The terminology is not intended to be limiting.

Method for Determining the Presence, amount or concentration of at Least One Autoantibody that Reacts with NGAL (or a Fragment Thereof) in a Test Sample

The present disclosure provides a method for determining the presence, amount or concentration of at least one autoantibody that reacts with NGAL (or a fragment thereof) in a test sample. As indicated previously herein, the presence of autoantibodies to NGAL (or fragments thereof) in a test sample can contribute to the generation of false negative results obtained in an assay. The method of the present disclosure enables one to determine the presence, amount or concentration of one or more autoantibodies in a test sample before, at the same time as, or after performing an assay for NGAL. The accuracy or reliability of results previously obtained with an assay for NGAL can be assessed with a method of determining the presence, amount or concentration of at least one autoantibody that reacts with NGAL (or a fragment thereof) in a test sample in accordance with the present disclosure.

The method can be performed in a homogeneous or heterogeneous format. It will be recognized by those skilled in the art that an essential difference between the two formats exists. For example, homogeneous formats lack one or more steps to separate a complex between an analyte of interest in a test sample and a specific binding partner for the analyte of interest from uncomplexed binding partners and other components of a test sample. Further, homogeneous assays employ detectable labels. One or more characteristics of the signal generated from the detectable label is/are modulated by the formation of a complex between the analyte of interest in the test sample (i.e., an autoantibody to NGAL (or a fragment thereof)) and a specific binding partner for the analyte of interest (e.g., NGAL (or a fragment thereof)). Examples of such homogeneous assays that can be used include, but are not limited to, fluorescence polarization immunoassay (FPIA), enzyme multiplied immunoassay technique (EMIT), bioluminescence resonance energy transfer (BRET), homogeneous chemiluminescent assay, etc. In a homogeneous format, after the test sample is obtained from a subject, a first mixture is prepared. The mixture contains the test sample being assessed for autoantibodies to NGAL (or a fragment thereof) and a first specific binding partner that is labeled with a detectable label. The first specific binding partner can be NGAL (or a fragment thereof).

Any suitable detectable label as is known in the art can be used. For example, a fluorescent label can be used in FPIA (see, e.g., U.S. Pat. Nos. 5,593,896, 5,573,904, 5,496,925, 5,359,093, and 5352803, which are hereby incorporated by reference in their entireties). An acridinium compound can be used as a detectable label in a homogeneous chemiluminescent assay (see, e.g., Adamczyk et al., Bioorg. Med. Chem. Lett. 16: 1324-1328 (2006); Adamczyk et al., Bioorg. Med. Chem. Lett. 4: 2313-2317 (2004); Adamczyk et al., Biorg. Med. Chem. Lett. 14: 3917-3921 (2004); and Adamczyk et al., Org. Lett. 5: 3779-3782 (2003)). Preferably, the acridinium compound is an acridinium-9-carboxamide. Specifically, the acridinium-9-carboxamide has a structure according to formula I:

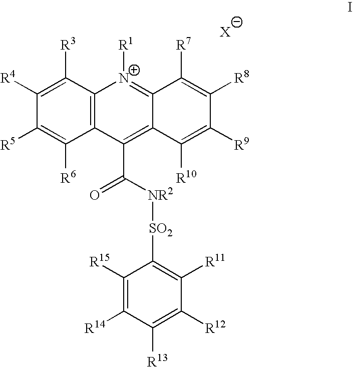

wherein R¹ and R² are each independently selected from the group consisting of: alkyl, alkenyl, alkynyl, aryl, aralkyl, sulfoalkyl, carboxyalkyl, oxoalkyl, and wherein R³ through R¹⁵ are each independently selected from the group consisting of: hydrogen, alkyl, alkenyl, alkynyl, aryl, aralkyl, amino, amido, acyl, alkoxyl, hydroxyl, carboxyl, halogen, halide, nitro, cyano, sulfo, sulfoalkyl, carboxyalkyl, and oxoalkyl; and further wherein any of the alkyl, alkenyl, alkynyl, aryl or aralkyl may contain one or more heteroatoms; and optionally, if present, x^{⊖} is an anion. Methods for preparing acridinium 9-carboxamides are described in Mattingly, J. Biolumin. Chemilumin. 6: 107-114 (1991); Adamczyk et al., J. Org. Chem. 63: 5636-5639 (1998); Adamczyk et al., Tetrahedron 55: 10899-10914 (1999); Adamczyk et al., Org. Lett. 1: 779-781 (1999); Adamczyk et al., Bioconjugate Chem. 11: 714-724 (2000); Mattingly et al., In Luminescence Biotechnology Instruments and Applications; Dyke, K. V. Ed.; CRC Press: Boca Raton, pp. 77-105 (2002); Adamczyk et al., Org. Lett. 5: 3779-3782 (2003); and U.S. Pat. Nos. 5,468,646, 5,543,524 and 5,783,699 (each of which is incorporated herein by reference in its entirety for its teachings regarding same).

Alternatively, the acridinium compound preferably is an acridinium-9-carboxylate aryl ester. The acridinium-9-carboxylate aryl ester has a structure according to formula II:

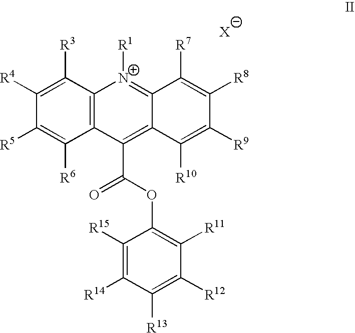

wherein R¹ is an alkyl, alkenyl, alkynyl, aryl, aralkyl, sulfoalkyl, carboxyalkyl, or oxoalkyl; wherein R³ through R¹⁵ are each independently selected from the group consisting of: hydrogen, alkyl, alkenyl, alkynyl, aryl, aralkyl, amino, amido, acyl, alkoxyl, hydroxyl, carboxyl, halogen, halide, nitro, cyano, sulfo, sulfoalkyl, carboxyalkyl, and oxoalkyl; and optionally, if present, x^{⊖} is an anion.

An example of an acridinium-9-carboxylate aryl ester of formula II is 10-methyl-9-(phenoxycarbonyl)acridinium fluorosulfonate (available from Cayman Chemical, Ann Arbor, Mich.). Methods for preparing acridinium 9-carboxylate aryl esters are described in McCapra et al., Photochem. Photobiol. 4: 1111-21 (1965); Razavi et al., Luminescence 15: 245-249 (2000); Razavi et al., Luminescence 15: 239-244 (2000); and U.S. Pat. No. 5,241,070 (each incorporated herein by reference in their entireties for their teachings regarding same). Such acridinium-9-carboxylate aryl esters are efficient chemiluminescent indicators for hydrogen peroxide produced in the oxidation of an analyte by at least one oxidase in terms of the intensity of the signal and/or the rapidity of the signal. The course of the chemiluminescent emission for the acridinium-9-carboxylate aryl ester is completed rapidly, i.e., in under 1 second, while the acridinium-9-carboxamide chemiluminescent emission extends over 2 seconds. Acridinium-9-carboxylate aryl ester, however, loses its chemiluminescent properties in the presence of protein. Therefore, its use requires the absence of protein during signal generation and detection. Methods for separating or removing proteins in the sample are well-known to those skilled in the art and include, but are not limited to, ultrafiltration, extraction, precipitation, dialysis, chromatography, and/or digestion (see, e.g., Wells, High Throughput Bioanalytical Sample Preparation. Methods and Automation Strategies, Elsevier (2003)). The amount of protein removed or separated from the test sample can be about 40%, about 45%, about 50%, about 55%, about 60%, about 65%, about 70%, about 75%, about 80%, about 85%, about 90%, or about 95%. Further details regarding acridinium-9-carboxylate aryl ester and its use are set forth in U.S. patent application Ser. No. 11/697,835, filed Apr. 9, 2007. Acridinium-9-carboxylate aryl esters can be dissolved in any suitable solvent, such as degassed anhydrous N,N-dimethylformamide (DMF) or aqueous sodium cholate.

With respect to the above formulae, “alkyl” means a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

“Alkenyl” means a straight or branched chain hydrocarbon containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

“Alkynyl” means a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

“Aryalkyl” means an aryl group, as defined herein, appended to the parent molecular moiety through an alkyl group, as defined herein. Representative examples of arylalkyl include, but are not limited to, benzyl, 2-phenylethyl, 3-phenylpropyl, and 2-naphth-2-ylethyl.

“Aryl” means a phenyl group, or a bicyclic or tricyclic fused ring system in which one or more of the fused rings is a phenyl group. Bicyclic fused ring systems are exemplified by a phenyl group fused to a cycloalkenyl group, as defined herein, a cycloalkyl group, as defined herein, or another phenyl group. Tricyclic fused ring systems are exemplified by a bicyclic fused ring system fused to a cycloalkenyl group, as defined herein, a cycloalkyl group, as defined herein, or another phenyl group. Representative examples of aryl include, but are not limited to, anthracenyl, azulenyl, fluorenyl, indanyl, indenyl, naphthyl, phenyl, and tetrahydronaphthyl. The aryl groups of the present invention can be optionally substituted with one, two, three, four, or five substituents independently selected from the group consisting of alkoxy, alkyl, carboxyl, halo, and hydroxyl.

“Cycloalkenyl” refers to a non-aromatic cyclic or bicyclic ring system having from three to ten carbon atoms and one to three rings, wherein each five-membered ring has one double bond, each six-membered ring has one or two double bonds, each seven- and eight-membered ring has one to three double bonds, and each nine- to ten-membered ring has one to four double bonds. Representative examples of cycloalkenyl groups include cyclohexenyl, octahydronaphthalenyl, norbornylenyl, and the like. The cycloalkenyl groups can be optionally substituted with one, two, three, four, or five substituents independently selected from the group consisting of alkoxy, alkyl, carboxyl, halo, and hydroxyl.

“Cycloalkyl” refers to a saturated monocyclic, bicyclic, or tricyclic hydrocarbon ring system having three to twelve carbon atoms. Representative examples of cycloalkyl groups include cyclopropyl, cyclopentyl, bicyclo[3.1.1]heptyl, adamantyl, and the like. The cycloalkyl groups of the present invention can be optionally substituted with one, two, three, four, or five substituents independently selected from the group consisting of alkoxy, alkyl, carboxyl, halo, and hydroxyl.

“Sulfoalkyl” refers to an alkyl group to which a sulfonate group is bonded, wherein the alkyl is bonded to the molecule of interest, whereas “carboxyalkyl” refers to an alkyl group that is substituted with one or more carboxy groups, “oxoalkyl” refers to an alkyl group that is substituted with one or more oxy groups, “amino” means—NRₐR_{b}, wherein Rₐ and R_{b} are independently selected from the group consisting of hydrogen, alkyl and alkylcarbonyl, “amido” means —C(O)NRₐR_{b}, wherein Rₐ and R_{b} are independently selected from the group consisting of hydrogen and alkyl, “acyl” means RC(O)—, “alkylcarbonyl,” means an alkyl group attached to the parent molecular moiety through a carbonyl group, “alkoxy” or “alkoxyl” means an alkyl group, as defined herein, appended to the parent molecular moiety through an oxygen atom, representative examples of which include, but are not limited to, methoxy, ethoxy, propoxy, 2-propoxy, butoxy, tert-butoxy, pentyloxy, and hexyloxy, “hydroxyl” means an —OH group, “carboxy” or “carboxyl” refers to —CO₂H, “halogen” means —Cl, —Br, —I or —F, “halide” means a binary compound, of which one part is a halogen atom and the other part is an element or radical that is less electronegative than the halogen, e.g., an alkyl radical, “nitro” means a —NO₂ group, “sulfo” means SO₃H, and “cyano” means a —CN group.

“Anion” refers to an anion of an inorganic or organic acid. Examples include, but are not limited to, hydrochloric acid, hydrobromic acid, sulfuric acid, methane sulfonic acid, formic acid, acetic acid, oxalic acid, succinic acid, tartaric acid, mandelic acid, fumaric acid, lactic acid, citric acid, glutamic acid, aspartic acid, phosphate, trifluoromethansulfonic acid, trifluoroacetic acid, fluorosulfonic acid, and any combinations thereof.

Chemiluminescent assays can be performed in accordance with the methods described in Adamczyk et al., Anal. Chim. Acta 579(1): 61-67 (2006).

While any suitable assay format can be used, a microplate chemiluminometer (Mithras LB-940, Berthold Technologies U.S.A., LLC, Oak Ridge, Tenn.) enables the assay of multiple samples of small volumes rapidly. The chemiluminometer can be equipped with multiple reagent injectors using 96-well black polystyrene microplates (Costar #3792). Each sample can be added into a separate well, followed by the simultaneous/sequential addition of other reagents as determined by the type of assay employed. Desirably, the formation of pseudobases in neutral or basic solutions employing an acridinium aryl ester is avoided, such as by acidification. The chemiluminescent response is then recorded well-by-well. In this regard, the time for recording the chemiluminescent response will depend, in part, on the delay between the addition of the reagents and the particular acridinium employed. For example, the emission of light from an acridinium carboxamide can be a pseudo-flash when the reagents are added in rapid succession, such as within 5 seconds, whereas the emission of light from an acridinium carboxamide can be a long-lived glow when there is a delay, such as 20 seconds, between the addition of a hydrogen peroxide-generating enzyme and the acridinium carboxamide.

The order in which the test sample and first specific binding partner labeled with the detectable label are added to form the mixture is not critical. After the first specific binding partner labeled with a detectable label and the test sample are added to form the first mixture, first specific binding partner-autoantibody complexes form.

Hydrogen peroxide can be generated in situ in the mixture or provided or supplied to the mixture before, simultaneously with, or after the addition of an above-described acridinium compound (specifically, the first specific binding partner labeled with the acridinium compound). Hydrogen peroxide can be generated in situ in a number of ways. For example, one or more hydrogen peroxide-generating enzymes can be added to the first mixture. The amount of one or more hydrogen peroxide-generating enzymes to be added to the mixture can be readily determined by one skilled in the art.

Hydrogen peroxide also can be generated electrochemically in situ (see, e.g., Agladze et al., J. Applied Electrochem. 37: 375-383 (2007); and Qiang et al., Water Research 36: 85-94 (2002)). Photochemical generation of hydrogen peroxide in situ is also possible (see, e.g., Draper et al., Archives of Environmental Contamination and Toxicology 12: 121-126 (1983)).

Alternatively, a source of hydrogen peroxide can be simply added to the mixture. For example, the source of the hydrogen peroxide can be one or more buffers or other solutions that are known to contain hydrogen peroxide. In this regard, a solution of hydrogen peroxide can simply be added.

Upon the addition of the acridinium, e.g., acridinium-9-carboxamide or acridinium-9-carboxylate aryl ester, and the simultaneous or subsequent addition of at least one basic solution to the sample, a detectable signal, namely, a chemiluminescent signal, indicative of the presence of autoantibody is generated. The basic solution contains at least one base and has a pH greater than or equal to 10, preferably, greater than or equal to 12. Examples of basic solutions include, but are not limited to, sodium hydroxide, potassium hydroxide, calcium hydroxide, ammonium hydroxide, magnesium hydroxide, sodium carbonate, sodium bicarbonate, calcium hydroxide, calcium carbonate, and calcium bicarbonate. The amount of basic solution added to the sample depends on the concentration of the basic solution. Based on the concentration of the basic solution used, one skilled in the art can easily determine the amount of basic solution to add to the sample.

The chemiluminescent signal that is generated can be detected using routine techniques known to those skilled in the art. Based on the intensity of the signal generated, the amount of autoantibody in the sample can be quantified. Specifically, the amount of autoantibody in the sample is proportional to the intensity of the signal generated. The amount of autoantibody present can be quantified by comparing the amount of light generated to a standard curve for an anti-NGAL antibody, such as an NGAL autoantibody, or by comparison to a reference standard. The standard curve can be generated using serial dilutions or solutions of known concentrations of an anti-NGAL antibody, such as an NGAL autoantibody, by mass spectroscopy, gravimetric methods, and other techniques known in the art.

In a heterogeneous format, after the test sample is obtained from a subject, a first mixture is prepared. The mixture contains the test sample being assessed for autoantibodies to NGAL (or a fragment thereof) and a first specific binding partner, wherein the first specific binding partner and any autoantibodies contained in the test sample form a first specific binding partner-autoantibody complex. Preferably, the first specific binding partner is NGAL or a fragment thereof. The order in which the test sample and the first specific binding partner are added to form the mixture is not critical. Preferably, the first specific binding partner is immobilized on a solid phase. The solid phase used in the immunoassay (for the first specific binding partner and, optionally, the second specific binding partner) can be any solid phase known in the art, such as, but not limited to, a magnetic particle, a bead, a test tube, a microtiter plate, a cuvette, a membrane, a scaffolding molecule, a film, a filter paper, a disc and a chip.

After the mixture containing the first specific binding partner-autoantibody complex is formed, any unbound autoantibodies are removed from the complex using any technique known in the art. For example, the unbound autoantibodies can be removed by washing.

After any unbound autoantibodies are removed, a second specific binding partner is added to the mixture to form a first specific binding partner-autoantibody-second specific binding partner complex. The second specific binding partner is preferably an anti-human antibody. Moreover, also preferably, the second specific binding partner is labeled with or contains a detectable label. In terms of the detectable label, any detectable label known in the art can be used. For example, the detectable label can be a radioactive label (such as ³H, ¹²⁵I, ³⁵S, ¹⁴C, ³²P, and ³³P), an enzymatic label (such as horseradish peroxidase, alkaline peroxidase, glucose 6-phosphate dehydrogenase, and the like), a chemiluminescent label (such as acridinium esters, thioesters, or sulfonamides; luminol, isoluminol, phenanthridinium esters, and the like), a fluorescent label (such as fluorescein (e.g., 5-fluorescein, 6-carboxyfluorescein, 3′6-carboxyfluorescein, 5(6)-carboxyfluorescein, 6-hexachloro-fluorescein, 6-tetrachlorofluorescein, fluorescein isothiocyanate, and the like)), rhodamine, phycobiliproteins, R-phycoerythrin, quantum dots (e.g., zinc sulfide-capped cadmium selenide), a thermometric label, or an immuno-polymerase chain reaction label. An introduction to labels, labeling procedures and detection of labels is found in Polak and Van Noorden, Introduction to Immunocytochemistry, 2nd ed., Springer Verlag, N.Y. (1997) and in Haugland, Handbook of Fluorescent Probes and Research Chemicals (1996), which is a combined handbook and catalogue published by Molecular Probes, Inc., Eugene, Oreg. Preferably, however, the detectable label is an acridinium compound that can be used in a chemiluminescent assay.

After the formation of the first specific binding partner-autoantibody-second specific binding complex, any unbound second specific binding partner (whether labeled or unlabeled) is removed from the complex using any technique known in the art. For example, the unbound second specific binding partner can be removed by washing.

Hydrogen peroxide can be generated in situ in the mixture or provided or supplied to the mixture before, simultaneously with, or after the addition of the above-described acridinium compound (specifically, the second specific binding partner labeled with the acridinium compound). Methods of generating hydrogen peroxide in situ are described above.

The timing and order in which the acridinium compound (specifically, the second specific binding partner labeled with the acridinium compound) and the hydrogen peroxide provided in or supplied to or generated in situ in the mixture is not critical. After the second specific binding partner labeled with a detectable label and the test sample are added to form the second mixture, first specific binding partner-autoantibody-second specific binding partner complexes form.

Upon the addition of the acridinium, e.g., acridinium-9-carboxamide or acridinium-9-carboxylate aryl ester, and the simultaneous or subsequent addition of at least one basic solution to the sample (as described above), a detectable signal, namely, a chemiluminescent signal, indicative of the presence of autoantibody is generated. Chemiluminescent signals generated can be detected using routine techniques known to those skilled in the art.

After any unbound second specific binding partner labeled with a detectable label is removed, a detectable signal from the detectable label is generated or emitted and then measured. Methods for generating signals from detectable labels and measuring the resulting signal generated are well-known to those skilled in the art. For example, a chemiluminescent signal can be generated after the addition of a basic solution. The amount of the autoantibodies in the test sample can be quantified based on the intensity of the signal generated. Specifically, the amount of autoantibodies contained in a test sample is proportional to the intensity of the signal generated. Specifically, the amount of autoantibodies present can be quantified based on comparing the amount of light generated to a standard curve for autoantibodies to a NGAL (or a fragment thereof) or by comparison to a reference standard. The standard curve can be generated using serial dilutions or solutions of an antibody to NGAL (or a fragment thereof) of known concentration, by mass spectroscopy, gravimetrically and by other techniques known in the art.

Accordingly, a method of determining the presence, amount or concentration of at least one autoantibody that reacts with NGAL or a fragment thereof in a test sample is provided. The method comprises assaying the test sample for at least one autoantibody that reacts with NGAL (or a fragment thereof). The assay employs NGAL (or a fragment thereof) and at least one detectable label. The assay comprises comparing a signal generated by the detectable label as a direct or indirect indication of the presence, amount or concentration of at least one autoantibody that reacts with NGAL (or a fragment thereof) in the test sample to a signal generated as a direct or indirect indication of the presence, amount or concentration of an antibody that reacts with NGAL (or a fragment thereof) in a control or calibrator. The calibrator is optionally part of a series of calibrators in which each of the calibrators differs from the other calibrators in the series by the concentration of an antibody that reacts with NGAL (or a fragment thereof). The method can be adapted for use in an automated system or a semi-automated system.

The method can comprise (i) contacting the test sample with NGAL (or a fragment thereof), which comprises a detectable label and binds to at least one autoantibody to form an NGAL (or a fragment thereof)/autoantibody complex, and

(ii) determining the presence, amount or concentration of at least one autoantibody, which reacts with NGAL (or a fragment thereof), in the test sample by detecting or measuring the signal generated by the detectable label in the NGAL (or fragment thereof)/autoantibody complex formed in (i). Preferably, the detectable label is an acridinium compound, such as an acridinium-9-carboxamide or an acridinium-9-carboxylate aryl ester.

The method can comprise (i) contacting the test sample with NGAL (or a fragment thereof), which binds to at least one autoantibody and which is optionally immobilized on a solid phase, so as to form an NGAL (or a fragment thereof)/autoantibody complex, (ii) contacting the NGAL (or a fragment thereof)/autoantibody complex with at least one detection antibody, which comprises a detectable label and binds to the autoantibody to form an NGAL (or a fragment thereof)/autoantibody/detection antibody complex, and (iii) determining the presence, amount or concentration of an autoantibody, which reacts with NGAL (or fragment thereof), in the test sample by detecting or measuring the signal generated by the detectable label in the NGAL (or a fragment thereof)/autoantibody/detection antibody complex formed in (ii). Optionally, the method further comprises removing any unbound at least one autoantibody after step (i) and removing any unbound at least one detection antibody after step (ii). Preferably, the detectable label is an acridinium compound, such as an acridinium-9-carboxamide or an acridinium-9-carboxylate aryl ester.

The method can further comprise determining the concentration of NGAL (or a fragment thereof) in the test sample. The method comprises assaying the test sample for NGAL (or a fragment thereof) by an assay employing at least one specific binding partner for NGAL and at least one detectable label and comprising comparing a signal generated by the detectable label as a direct or indirect indication of the concentration of NGAL (or a fragment thereof) in the test sample to a signal generated as a direct or indirect indication of the concentration of NGAL in a control or calibrator, which is optionally part of a series of calibrators in which each of the calibrators differs from the other calibrators in the series by the concentration of NGAL.

For example, the concentration of NGAL (or a fragment thereof) in a test sample can be determined by an assay as described above with respect to NGAL autoantibody or by immunoassay using any format known in the art, such as, but not limited to, sandwich immunoassay (e.g., monoclonal-polyclonal sandwich immunoassays, including radioisotope detection (radioimmunoassay (RIA)) and enzyme detection (enzyme immunoassay (EIA) or ELISA (e.g., Quantikine ELISA assays, R&D Systems, Minneapolis, Minn.)), competitive inhibition immunoassay (e.g., forward and reverse), and fluorescence polarization immunoassay (FPIA). A chemiluminescent microparticle immunoassay, in particular one employing the ARCHITECT® automated analyzer (Abbott Laboratories, Abbott Park, Ill.), is an example of a preferred immunoassay.

Monoclonal and polyclonal antibodies (mAbs and pAbs, respectively) can be produced for use in immunoassays in accordance with methods known in the art. NGAL, such as recombinantly produced NGAL, in particular, recombinantly produced human NGAL, such as in a composition comprising an adjuvant, can be injected into a host animal, such as a rabbit, goat, mouse, guinea pig, or horse, at one or more sites. Further injections are made at the same or other sites at regular or irregular intervals thereafter with bleedings being taken to assess antibody titer until it is determined that optimal titer has been reached. The antibodies are obtained by either bleeding the host animal to yield a volume of antiserum, or by somatic cell hybridization techniques or other techniques known in the art. For example, the antibody-producing cells can be fused by standard somatic cell fusion procedures with immortalizing cells, such as myeloma cells, to yield hybridoma cells. Such techniques are well-known in the art, and include, for example, the hybridoma technique as originally developed by Kohler and Milstein, Nature 256: 495-497 (1975)), the human B cell hybridoma technique (Kozbar et al., Immunology Today 4: 72 (1983)), and the EBV-hybridoma technique to produce human mAbs (Cole et al., Monoclonal Antibodies and Cancer Therapy, Alan R. Liss, Inc. pp. 77-96 (1985)). The technology for producing mAb hybridomas is well-known to those skilled in the art (see, e.g., Kenneth, in Monoclonal Antibodies: A New Dimension in Biological Analyses, Plenum Pub. Corp., New York (1980)). Alternatively, anti-NGAL antibodies can be commercially obtained from any one of a number of sources, such as R&D Systems (Minneapolis, Minn.) among others.

In a sandwich immunoassay format, typically at least two antibodies are used to separate and quantify an analyte of interest, in this case NGAL (or a fragment thereof). More specifically, the two antibodies bind to different epitopes on the analyte of interest, thereby forming what is referred to as a “sandwich,” i.e., antibody-analyte-antibody. One or more antibodies, which bind(s) to the analyte of interest and is/are typically bound to a substrate before or after contact with the analyte of interest, is/are referred to as the “capture antibody” or “capture antibodies,” whereas one or more other antibodies, which is/are labeled and bind(s) to the analyte bound by the capture antibody, is/are referred to as the “detection antibody,” “detection antibodies,” “conjugate,” or “conjugates.” Preferably, the binding of one antibody to the analyte does not interfere with the binding of any other antibody to the analyte. Also, preferably, at least the capture antibody is present in a molar excess amount of the maximum amount of the analyte, i.e., NGAL (or fragment thereof), expected to be present in a sample. While the detection antibody is typically labeled prior to contact with the analyte-capture antibody complex, the detection antibody can be labeled simultaneously with or subsequently to the formation of the analyte-capture antibody complex.

Generally speaking, a test sample being assayed for (for example, suspected of containing) NGAL or a fragment thereof can be contacted with at least one capture antibody (or antibodies) and at least one detection antibody (which is either a second detection antibody or a third detection antibody) either simultaneously or sequentially and in any order. For example, the test sample can be first contacted with at least one capture antibody and then (sequentially) with at least one detection antibody. Alternatively, the test sample can be first contacted with at least one detection antibody and then (sequentially) with at least one capture antibody. In yet another alternative, the test sample can be contacted simultaneously with a capture antibody and a detection antibody.

In the sandwich assay format, a test sample suspected of containing NGAL or a fragment thereof is first brought into contact with an at least one first capture antibody under conditions, which allow the formation of a first antibody/NGAL (or a fragment thereof) complex. If more than one capture antibody is used, a first multiple capture antibody/NGAL (or a fragment thereof) complex is formed. In a sandwich assay, the antibodies, preferably, the at least one capture antibody, are used in molar excess amounts of the maximum amount of NGAL (or a fragment thereof) expected in the test sample. For example, from about 5 μg/mL to about 1 mg/mL of antibody per mL of buffer (e.g., microparticle coating buffer) can be used.

Competitive inhibition immunoassays, which are often used to measure small analytes because binding by only one antibody is required, comprise sequential and classic formats. In a sequential competitive inhibition immunoassay a capture mAb to an analyte of interest is coated onto a well of a microtiter plate. When the sample containing the analyte of interest is added to the well, the analyte of interest binds to the capture mAb. After washing, a known amount of labeled (e.g., biotin or horseradish peroxidase (HRP)) analyte is added to the well. A substrate for an enzymatic label is necessary to generate a signal. An example of a suitable substrate for HRP is 3,3′,5,5′-tetramethylbenzidine (TMB). After washing, the signal generated by the labeled analyte is measured and is inversely proportional to the amount of analyte in the sample. In a classic competitive inhibition immunoassay an mAb to an analyte of interest is coated onto a well of a microtiter plate. However, unlike the sequential competitive inhibition immunoassay, the sample and the labeled analyte are added to the well at the same. Any analyte in the sample competes with labeled analyte for binding to the capture mAb. After washing, the signal generated by the labeled analyte is measured and is inversely proportional to the amount of analyte in the sample.

Optionally, prior to contacting the test sample with the at least one capture antibody (for example, the first capture antibody), the at least one capture antibody can be bound to a solid support, which facilitates the separation of the first antibody/NGAL (or a fragment thereof) complex from the test sample. The substrate to which the capture antibody is bound can be any suitable solid support or solid phase that facilitates separation of the capture antibody-analyte complex from the sample. Examples include a well of a plate, such as a microtiter plate, a test tube, a porous gel (e.g., silica gel, agarose, dextran, or gelatin), a polymeric film (e.g., polyacrylamide), beads (e.g., polystyrene beads or magnetic beads), a strip of a filter/membrane (e.g., nitrocellulose or nylon), microparticles (e.g., latex particles, magnetizable microparticles (e.g., microparticles having ferric oxide or chromium oxide cores and homo- or hetero-polymeric coats and radii of about 1-10 microns), sheep red blood cells, or DURACYTES® (Abbott Laboratories), which are red blood cells that have been “fixed” by pyruvic aldehyde and formaldehyde)). The substrate can comprise a suitable porous material with a suitable surface affinity to bind antigens and sufficient porosity to allow access by detection antibodies. A microporous material is generally preferred, although a gelatinous material in a hydrated state can be used. Such porous substrates are preferably in the form of sheets having a thickness of about 0.01 to about 0.5 mm, preferably about 0.1 mm. While the pore size may vary quite a bit, preferably the pore size is from about 0.025 to about 15 microns, more preferably from about 0.15 to about 15 microns. The surface of such substrates can be activated by chemical processes that cause covalent linkage of an antibody to the substrate. Irreversible binding, generally by adsorption through hydrophobic forces, of the antigen or the antibody to the substrate results; alternatively, a chemical coupling agent or other means can be used to bind covalently the antibody to the substrate, provided that such binding does not interfere with the ability of the antibody to bind IL-18. Alternatively, the antibody can be bound with microparticles, which have been previously coated with streptavidin or biotin (e.g., using Power-Bind™-SA-MP streptavidin-coated microparticles (Seradyn, Indianapolis, Ind.)) or anti-species-specific mAbs. If necessary, the substrate can be derivatized to allow reactivity with various functional groups on the antibody. Such derivatization requires the use of certain coupling agents, examples of which include, but are not limited to, maleic anhydride, N-hydroxysuccinimide, and 1-ethyl-3-(3-dimethylaminopropyl)carbodiimide.

After the test sample being assayed for NGAL (or a fragment thereof) is brought into contact with the at least one capture antibody (for example, the first capture antibody), the mixture is incubated in order to allow for the formation of a first antibody (or multiple antibody)-NGAL (or a fragment thereof) complex. The incubation can be carried out at a pH of from about 4.5 to about 10.0, at a temperature of from about 2° C. to about 45° C., and for a period from at least about one (1) minute to about eighteen (18) hours, preferably from about 1 to about 24 minutes, most preferably for about 4 to about 18 minutes. The immunoassay described herein can be conducted in one step (meaning the test sample, at least one capture antibody and at least one detection antibody are all added sequentially or simultaneously to a reaction vessel) or in more than one step, such as two steps, three steps, etc.

After formation of the (first or multiple) capture antibody/NGAL (or a fragment thereof) complex, the complex is then contacted with at least one detection antibody (under conditions which allow for the formation of a (first or multiple) capture antibody/NGAL (or a fragment thereof)/second antibody detection complex). The at least one detection antibody can be the second, third, fourth, etc. antibodies used in the immunoassay. If the capture antibody/NGAL (or a fragment thereof) complex is contacted with more than one detection antibody, then a (first or multiple) capture antibody/NGAL (or a fragment thereof)/(multiple) detection antibody complex is formed. As with the capture antibody (e.g., the first capture antibody), when the at least second (and subsequent) detection antibody is brought into contact with the capture antibody/NGAL (or a fragment thereof) complex, a period of incubation under conditions similar to those described above is required for the formation of the (first or multiple) capture antibody/NGAL (or a fragment thereof)/(second or multiple) detection antibody complex. Preferably, at least one detection antibody contains a detectable label. The detectable label can be bound to the at least one detection antibody (e.g., the second detection antibody) prior to, simultaneously with, or after the formation of the (first or multiple) capture antibody/NGAL (or a fragment thereof)/(second or multiple) detection antibody complex. Any detectable label known in the art can be used (see discussion above, including Polak and Van Noorden (1997) and Haugland (1996)).

The detectable label can be bound to the antibodies either directly or through a coupling agent. An example of a coupling agent that can be used is EDAC (1-ethyl-3-(3-dimethylaminopropyl) carbodiimide, hydrochloride), which is commercially available from Sigma-Aldrich, St. Louis, Mo. Other coupling agents that can be used are known in the art. Methods for binding a detectable label to an antibody are known in the art. Additionally, many detectable labels can be purchased or synthesized that already contain end groups that facilitate the coupling of the detectable label to the antibody, such as CPSP-Acridinium Ester or SPSP-Acridinium Ester.

The (first or multiple) capture antibody/NGAL (or a fragment thereof)/(second or multiple) detection antibody complex can be, but does not have to be, separated from the remainder of the test sample prior to quantification of the label. For example, if the at least one capture antibody (e.g., the first capture antibody) is bound to a solid support, such as a well or a bead, separation can be accomplished by removing the fluid (of the test sample) from contact with the solid support. Alternatively, if the at least first capture antibody is bound to a solid support, it can be simultaneously contacted with the NGAL (or a fragment thereof)-containing sample and the at least one second detection antibody to form a first (multiple) antibody/NGAL (or a fragment thereof)/second (multiple) antibody complex, followed by removal of the fluid (test sample) from contact with the solid support. If the at least one first capture antibody is not bound to a solid support, then the (first or multiple) capture antibody/NGAL (or a fragment thereof)/(second or multiple) detection antibody complex does not have to be removed from the test sample for quantification of the amount of the label.

After formation of the labeled capture antibody/NGAL (or a fragment thereof)/detection antibody complex (e.g., the first capture antibody/NGAL (or a fragment thereof)/second detection antibody complex), the amount of label in the complex is quantified using techniques known in the art. For example, if an enzymatic label is used, the labeled complex is reacted with a substrate for the label that gives a quantifiable reaction, such as the development of color. If the label is a radioactive label, the label is quantified using a scintillation counter. If the label is a fluorescent label, the label is quantified by stimulating the label with a light of one color (which is known as the “excitation wavelength”) and detecting another color (which is known as the “emission wavelength”) that is emitted by the label in response to the stimulation. If the label is a chemiluminescent label, the label is quantified detecting the light emitted either visually or by using luminometers, x-ray film, high-speed photographic film, a CCD camera, etc. Once the amount of the label in the complex has been quantified, the concentration of NGAL (or a fragment thereof) in the test sample is determined by use of a standard curve that has been generated using serial dilutions of NGAL (or a fragment thereof) of known concentration. Other than using serial dilutions of NGAL (or a fragment thereof), the standard curve can be generated gravimetrically, by mass spectroscopy and by other techniques known in the art.

The NGAL assay can employ a monoclonal antibody (mAb) sandwich that utilizes a capture antibody that binds only free NGAL and excludes bound NGAL, such as NGAL bound to metalloproteinase-9 (MMP-9) or gelatinase B. The amount of captured free NGAL can be detected with an acridinylated anti-NGAL mAb.

FPIAs are based on competitive binding immunoassay principles. A fluorescently labeled compound, when excited by a linearly polarized light, will emit fluorescence having a degree of polarization inversely proportional to its rate of rotation. When a fluorescently labeled tracer-antibody complex is excited by a linearly polarized light, the emitted light remains highly polarized because the fluorophore is constrained from rotating between the time light is absorbed and the time light is emitted. When a “free” tracer compound (I.e., a compound that is not bound to an antibody) is excited by linearly polarized light, its rotation is much faster than the corresponding tracer-antibody conjugate produced in a competitive binding immunoassay. FPIAs are advantageous over RIAs inasmuch as there are no radioactive substances requiring special handling and disposal. In addition, FPIAs are homogeneous assays that can be easily and rapidly performed.

The method can further comprise diagnosing, prognosticating, or assessing the efficacy of a therapeutic/prophylactic treatment of a patient from whom the test sample was obtained. If the method further comprises assessing the efficacy of a therapeutic/prophylactic treatment of the patient from whom the test sample was obtained, the method optionally further comprises modifying the therapeutic/prophylactic treatment of the patient as needed to improve efficacy.

The methods described herein can be used for determining the reliability of a result obtained from an assay (e.g., namely, an assay that was previously, simultaneously or subsequently performed) for detecting or quantifying the amount of NGAL in a test sample obtained from a subject. Specifically, such a method involves obtaining a test sample (a single test sample can be obtained from a subject and split between the autoantibody assay and the NGAL assay) from the same subject and determining the presence or the concentration of at least one autoantibody that reacts with NGAL using any of the assays described herein or any alternate assay, which may or may not be an immunoassay, for an autoantibody as known in the art. If the concentration of the at least one autoantibody is elevated compared to a predetermined level, then the concentration of NGAL as determined by separate assay is considered not to be reliable. However, if the concentration of the at least one autoantibody is lower or the same as a predetermined level, then the concentration of NGAL as determined by separate assay is considered to be reliable.

Generally, a predetermined level can be employed as a benchmark against which to assess results obtained upon assaying a test sample for at least one autoantibody that reacts with NGAL or a fragment thereof. Generally, in making such a comparison, the predetermined level is obtained by running a particular assay a sufficient number of times and under appropriate conditions such that a linkage or association of analyte (e.g., autoantibody) presence, amount or concentration with a particular stage or endpoint of a disease, disorder or condition (e.g., cardiovascular disease or renal disease) or with particular clinical indicia can be made. Typically, the predetermined level is obtained with assays of reference subjects (or populations of subjects).

With respect to at least one autoantibody reactive with NGAL (or a fragment thereof), it is envisioned that such autoantibodies can be directed against a variety of in vivo targets associated with the cardiovascular system or other major organ systems, such as the excretory system (e.g., the kidneys), in which NGAL has any function or impact. Accordingly, a particular autoantibody may either or increase or decrease with respect to a predetermined level.

In particular, with respect to a predetermined level as employed for monitoring disease progression and/or treatment, the concentration or amount of an autoantibody reactive with NGAL or fragment thereof may be either “unchanged,” “favorable” (or “favorably altered”), or “unfavorable” (or “unfavorably altered”). Generally, because autoantibodies as described herein appear to be associated with an NGAL-specific cardiophysiopathology and are elevated in a low proportion (e.g., less than about five %, especially from about 0.5% to about 5%) of the so-called normal population and are elevated in a higher proportion (less than about 15%, especially from about 10 to about 15%) of the population testing positive for the presence of NGAL, it is likely in most cases that “unfavorable” (“unfavorably altered”) corresponds to an increase or elevation in autoantibody amount or concentration, and “favorable” (“favorably altered”) corresponds to an decrease or reduction in autoantibody amount or concentration, in each case relative to a predetermined level or to a prior measured value.

As used herein, the term “elevated” or “increased” refers to a concentration or amount in a test sample that is higher than a typical or normal level or range (e.g., predetermined level), or is higher that another reference level or range (e.g., earlier or baseline sample). The term “lowered” or “reduced” refers to a concentration or amount in a test sample that is higher than a typical or normal level or range (e.g., predetermined level), or is higher that another reference level or range (e.g., earlier or baseline sample). The term “altered” refers to a concentration or amount in a sample that is altered (increased or decreased) over a typical or normal level or range (e.g., predetermined level), or over another reference level or range (e.g., earlier or baseline sample).

The typical or normal level or range for NGAL and autoantibodies reactive therewith is defined in accordance with standard practice. Because the levels of autoantibodies in some instances will be very low, a so-called altered level or alteration can be considered to have occurred when there is any net change as compared to the typical or normal level or range, or reference level or range, that cannot be explained by experimental error or sample variation. Thus, the level measured in a particular sample will be compared with the level or range of levels determined in similar samples from a so-called normal subject. In this context, a “normal subject” is an individual with no detectable cardiovascular or renal pathology, for example, and a “normal” (sometimes termed “control”) patient or population is/are one(s) that exhibits no detectable cardiovascular or renal pathology, for example. Furthermore, given that one or more autoantibodies against NGAL are not routinely found at high levels in the majority of the human population, a “normal subject” can be considered an individual with no substantial detectable increased or elevated concentration or amount of NGAL autoantibodies, and a “normal” (sometimes termed “control”) patient or population is/are one(s) that exhibits no substantial detectable increased or elevated concentration or amount of NGAL autoantibodies. An “apparently normal subject” is one in which autoantibodies have not been or are being assessed. The level of an analyte is said to be “elevated” when the analyte is normally undetectable (e.g., the normal level is zero, or within a range of from about 25 to about 75 percentiles of normal populations), but is detected in a test sample, as well as when the analyte is present in the test sample at a higher than normal level.

As previously mentioned, it is believed that one or more autoantibodies reactive with NGAL or a fragment thereof as described herein can be directed against a variety of in vivo targets associated with the cardiovascular system or other major organ systems (e.g., excretory system, in particular the kidneys) in which NGAL has any function or impact. Thus, inter alia, the disclosure provides a method of screening for a subject having, or at risk of having, cardiovascular disease or renal disease, for example, as defined herein.

Any of the test methods as described herein can be performed in conjunction with one or more other tests including, but not limited to, physical examination, and/or the taking of a medical history to allow a differential diagnosis of cardiovascular or renal disease. The various tests and parameters employed in diagnosing these disorders are well-known to those of skill in the art. Furthermore, any of the methods can be carried out on samples from asymptomatic subjects or subjects having one or more risk factors associated with, or symptoms of, cardiovascular disease or renal disease.

In particular embodiments, when a subject is determined to have an unfavorable level of one or more autoantibodies to NGAL, the subject optionally is assessed for one or more additional indicators of cardiovascular disease, such as myoglobin, CK-MB (creatine kinase muscle-brain), BNP (brain natriuretic peptide), CRP (C reactive protein), cardiac troponin I (cTnI), cardiac troponin T (cTnT), blood oxygen level, cardiac imaging, electrocardiography, and any others that are known in the art. Alternatively or additionally, the subject optionally is assessed for one or more additional indicators of renal disease, such as proteinuria, heamaturia, serum creatine, cystatin C, S-adenosylhomocysteine, homocysteine, an abnormally high body mass index (BMI), obesity, and others as known in the art.

However, such testing optionally can be carried out even when there has been no prior detection of an unfavorable level of one or more autoantibodies to NGAL (or a fragment thereof). For example, even in the absence of detection of at least one autoantibody that reacts with NGAL (or fragment thereof), any of the methods described herein can be used in conjunction with the measurement of one or more markers associated with cardiovascular disease or renal disease. Markers for cardiovascular disease include a natriuretic peptide or fragment thereof (e.g., combination antigen/antibody assay, or independent tests), as well as (and not limited to) pregnancy-associated plasma protein A (PAPP-A), interleukin 8 (IL-8), IL-10, interleukin-18 (IL-18/IL-18b), ischemic modified albumin (IMA), ICAM-1 (intercellular cell adhesion molecule-1), VCAM-1 (vascular cell adhesion molecule-1), fatty acid binding protein (FABP), E-selectin, P-selectin, fibrinogen, serum amyloid A (SAA), MPO (myeloperoxidase), LpPLA2 (lipoprotein-associated phospholipase A2), GP-BB (glycogen phosphorylase isoenzyme BB), ILIRA, TAFI (thrombin activable fibrinolysis inhibitor), soluble fibrin, anti-oxLDL (antibodies against oxidized low density lipoprotein), MCP-1 (monocyte chemoattractant protein-1), procoagulant tissue factor (TF), MMP-9 (matrix metalloproteinase 9), Ang-2 (angiopoietin-2), bFGF (basic fibroblast growth factor), VLDL (very low density lipoprotein), and PAI-1 (plasminogen activator inhibitor-1), among others.

Thus, in one embodiment provided herein is a method for determining the reliability of a result obtained from a separate assay that was previously, simultaneously or subsequently performed for detecting or quantifying the amount or concentration of NGAL in a test sample obtained from a subject, wherein the method comprises:

(a) determining the amount or the concentration in the test sample of at least one autoantibody that reacts with NGAL; and

(b) comparing the amount or the concentration in (a) to a predetermined level, wherein if the amount or the concentration is elevated as compared to a predetermined level, then the amount or concentration of NGAL as determined by separate assay is considered not to be reliable, and wherein if the amount or the concentration in (a) is lower or the same as a predetermined level, then the amount or concentration of NGAL as determined by separate assay is considered to be reliable.

Accordingly, the methods described herein also can be used to determine whether or not a subject has or is at risk of developing a cardiovascular disease or a renal disease. Specifically, such a method can comprise the steps of:

(a) determining the concentration or amount in a test sample from a subject of at least one autoantibody that reacts with NGAL (or a fragment thereof) (e.g., using the methods described herein, or methods known in the art); and

(b) comparing the concentration or amount of at least one autoantibody that reacts with NGAL (or fragment thereof) determined in step (a) with a predetermined level, wherein, if the concentration or amount of the one or more autoantibodies reactive with human NGAL determined in step (a) is favorable with respect to a predetermined level, then the subject is determined not to have or be at risk for a cardiovascular disease or renal disease. However, if the concentration or amount of the one or more autoantibodies reactive with NGAL determined in step (a) is unfavorable with respect to the predetermined level, then the subject is determined to have or be at risk for a cardiovascular disease or renal disease.

Additionally, provided herein is method of monitoring the progression of disease in a subject. Optimally the method comprising the steps of:

(a) determining the concentration or amount in a test sample from a subject of at least one autoantibody reactive with NGAL;

(b) determining the concentration or amount in a later test sample from the subject of at least one autoantibody reactive with NGAL; and

(c) comparing the concentration or amount of at least one autoantibody reactive with NGAL as determined in step (b) with the concentration or amount of at least one autoantibody reactive with NGAL determined in step (a), wherein if the concentration or amount determined in step (b) is unchanged or is unfavorable when compared to the concentration or amount of at least one autoantibody reactive with NGAL determined in step (a), then the disease in the subject is determined to have continued, progressed or worsened. By comparison, if the concentration or amount of at least one autoantibody reactive with NGAL determined in step (b) is favorable when compared to the concentration or amount of at least one autoantibody reactive with NGAL as determined in step (a), then the disease in the subject is determined to have discontinued, regressed or improved.

Optionally, the method further comprises comparing the concentration or amount of at least one autoantibody reactive with NGAL determined in step (b), for example, with a predetermined level. Further, optionally the method comprises treating the subject with one or more pharmaceutical compositions for a period of time if the comparison shows that the concentration or amount of at least one autoantibody reactive with NGAL as determined in step (b), for example, is unfavorably altered with respect to the predetermined level.

Still further, the methods can be used to monitor treatment in a subject receiving treatment with one or more pharmaceutical compositions. Specifically, such methods involve providing a first test sample from a subject before the subject has been administered one or more pharmaceutical compositions. Next, the concentration or amount in a first test sample from a subject of at least one autoantibody reactive with NGAL is determined (e.g., using the methods described herein or as known in the art). After the concentration or amount of at least one autoantibody reactive with NGAL is determined, optionally the concentration or amount of at least one autoantibody reactive with NGAL is then compared with a predetermined level. If the concentration or amount of the at least one autoantibody reactive with NGAL as determined in the first test sample is lower than the predetermined level, then the subject is not treated with one or more pharmaceutical compositions. However, if the concentration or amount of the at least one autoantibody reactive with NGAL as determined in the first test sample is higher than the predetermined level, then the subject is treated with one or more pharmaceutical compositions for a period of time. The period of time that the subject is treated with the one or more pharmaceutical compositions can be determined by one skilled in the art (for example, the period of time can be from about seven (7) days to about two years, preferably from about fourteen (14) days to about one (1) year).

During the course of treatment with the one or more pharmaceutical compositions, second and subsequent test samples are then obtained from the subject. The number of test samples and the time in which said test samples are obtained from the subject are not critical. For example, a second test sample could be obtained seven (7) days after the subject is first administered the one or more pharmaceutical compositions, a third test sample could be obtained two (2) weeks after the subject is first administered the one or more pharmaceutical compositions, a fourth test sample could be obtained three (3) weeks after the subject is first administered the one or more pharmaceutical compositions, a fifth test sample could be obtained four (4) weeks after the subject is first administered the one or more pharmaceutical compositions, etc.

After each second or subsequent test sample is obtained from the subject, the concentration or amount of at least one autoantibody reactive with NGAL is determined in the second or subsequent test sample is determined (e.g., using the methods described herein or as known in the art). The concentration or amount of at least one autoantibody reactive with NGAL as determined in each of the second and subsequent test samples is then compared with the concentration or amount of at least one autoantibody reactive with NGAL as determined in the first test sample (e.g., the test sample that was originally optionally compared to the predetermined level). If the concentration or amount of at least one autoantibody reactive with NGAL as determined in step (c) is favorable when compared to the concentration or amount of at least one autoantibody reactive with NGAL as determined in step (a), then the disease in the subject is determined to have discontinued, regressed or improved, and the subject should continue to be administered the one or pharmaceutical compositions of step (b). However, if the concentration or amount determined in step (c) is unchanged or is unfavorable when compared to the concentration or amount of at least one autoantibody reactive with NGAL determined in step (a), then the disease in the subject is determined to have continued, progressed or worsened, and the subject should be treated with a higher concentration of the one or more pharmaceutical compositions administered to the subject in step (b) or the subject should be treated with one or more pharmaceutical compositions that are different from the one or more pharmaceutical compositions administered to the subject in step (b). Specifically, the subject can be treated with one or more pharmaceutical compositions that are different from the one or more pharmaceutical compositions that the subject had previously received to decrease or lower said subject's NGAL autoantibodies levels.

Generally, for assays in which repeat testing may be done (e.g., monitoring disease progression and/or response to treatment), a second or subsequent test sample is obtained at a period in time after the first test sample has been obtained from the subject. Specifically, a second test sample from the subject can be obtained minutes, hours, days, weeks or years after the first test sample has been obtained from the subject. For example, the second test sample can be obtained from the subject at a time period of about 1 minute, about 5 minutes, about 10 minutes, about 15 minutes, about 30 minutes, about 45 minutes, about 60 minutes, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days, about 4 days, about 5 days, about 6 days, about 7 days, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, about 27 weeks, about 28 weeks, about 29 weeks, about 30 weeks, about 31 weeks, about 32 weeks, about 33 weeks, about 34 weeks, about 35 weeks, about 36 weeks, about 37 weeks, about 38 weeks, about 39 weeks, about 40 weeks, about 41 weeks, about 42 weeks, about 43 weeks, about 44 weeks, about 45 weeks, about 46 weeks, about 47 weeks, about 48 weeks, about 49 weeks, about 50 weeks, about 51 weeks, about 52 weeks, about 1.5 years, about 2 years, about 2.5 years, about 3.0 years, about 3.5 years, about 4.0 years, about 4.5 years, about 5.0 years, about 5.5 years, about 6.0 years, about 6.5 years, about 7.0 years, about 7.5 years, about 8.0 years, about 8.5 years, about 9.0 years, about 9.5 years or about 10.0 years after the first test sample from the subject is obtained. When used to monitor disease progression, the above assay can be used to monitor the progression of disease in subjects suffering from acute conditions. Acute conditions, also known as critical care conditions, refer to acute, life-threatening diseases or other critical medical conditions involving, for example, the cardiovascular system or excretory system. Typically, critical care conditions refer to those conditions requiring acute medical intervention in a hospital-based setting (including, but not limited to, the emergency room, intensive care unit, trauma center, or other emergent care setting) or administration by a paramedic or other field-based medical personnel. For critical care conditions, repeat monitoring is generally done within a shorter time frame, namely, minutes, hours or days (e.g., about 1 minute, about 5 minutes, about 10 minutes, about 15 minutes, about 30 minutes, about 45 minutes, about 60 minutes, about 2 hours, about 3 hours, about 4 hours, 4 about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days, about 4 days, about 5 days, about 6 days or about 7 days), and the initial assay likewise is generally done within a shorter timeframe, e.g., about minutes, hours or days of the onset of the disease or condition.

The assays also can be used to monitor the progression of disease in subjects suffering from chronic or non-acute conditions. Non-critical care or, non-acute conditions, refers to conditions other than acute, life-threatening disease or other critical medical conditions involving, for example, the cardiovascular system and/or excretory system. Typically, non-acute conditions include those of longer-term or chronic duration. For non-acute conditions, repeat monitoring generally is done with a longer timeframe, e.g., hours, days, weeks, months or years (e.g., about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days, about 4 days, about 5 days, about 6 days, about 7 days, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, about 27 weeks, about 28 weeks, about 29 weeks, about 30 weeks, about 31 weeks, about 32 weeks, about 33 weeks, about 34 weeks, about 35 weeks, about 36 weeks, about 37 weeks, about 38 weeks, about 39 weeks, about 40 weeks, about 41 weeks, about 42 weeks, about 43 weeks, about 44 weeks, about 45 weeks, about 46 weeks, about 47 weeks, about 48 weeks, about 49 weeks, about 50 weeks, about 51 weeks, about 52 weeks, about 1.5 years, about 2 years, about 2.5 years, about 3.0 years, about 3.5 years, about 4.0 years, about 4.5 years, about 5.0 years, about 5.5 years, about 6.0 years, about 6.5 years, about 7.0 years, about 7.5 years, about 8.0 years, about 8.5 years, about 9.0 years, about 9.5 years or about 10.0 years), and the initial assay likewise generally is done within a longer time frame, e.g., about hours, days, months or years of the onset of the disease or condition.

Furthermore, the above assays can be performed using a first test sample obtained from a subject where the first test sample is whole blood, serum, or plasma. The above assays can then be repeated using a second test sample obtained from the subject where the second test sample is something other than whole blood, serum or plasma (e.g., urine). The results obtained from the assays using the first test sample and the second test sample can be compared. The comparison can be used to assess the status of a disease or condition in the subject.

Moreover, the present disclosure also relates to methods of determining whether a subject predisposed to or suffering from a disease (e.g., cardiovascular disease or renal disease) will benefit from treatment. In particular, the disclosure relates to NGAL companion diagnostic methods and products. Thus, the method of “monitoring the treatment of disease in a subject” as described herein further optimally also can encompass selecting or identifying candidates for therapy.

Thus, in particular embodiments, the disclosure also provides a method of determining whether a subject having, or at risk for, cardiovascular or renal disease is a candidate for therapy. Generally, the subject is one who has experienced some symptom of cardiovascular disease or who has actually been diagnosed as having, or being at risk for, cardiovascular or renal disease, and/or who demonstrates an unfavorable concentration or amount of at least one autoantibody reactive with NGAL or a fragment thereof, as described herein.

The method optionally comprises an assay as described herein, where analyte is assessed before and following treatment of a subject with one or more pharmaceutical compositions (e.g., particularly with a pharmaceutical related to a mechanism of action involving NGAL), with immunosuppressive therapy, or by immunoabsorption therapy, or where analyte is assessed following such treatment and the concentration or the amount of analyte is compared against a predetermined level. An unfavorable concentration of amount of analyte observed following treatment confirms that the subject will not benefit from receiving further or continued treatment, whereas a favorable concentration or amount of analyte observed following treatment confirms that the subject will benefit from receiving further or continued treatment. This confirmation assists with management of clinical studies, and provision of improved patient care.

It goes without saying that while certain embodiments herein are advantageous when employed to assess cardiovascular or renal disease, the assays and kits also optionally can be employed to assess NGAL autoantibodies in other diseases, disorders and conditions, e.g., cancer, sepsis, and any disease, disorder or condition that might involve an assessment of NGAL or autoantibody thereto.

More specifically, in addition to assessment of renal disorders, diseases and injuries (see, e.g., U.S. Pat. App. Pub. Nos. 2008/0090304, 2008/0014644, 2008/0014604, 2007/0254370, and 2007/0037232), the assay and assay components as described herein optionally can also be employed in any other NGAL or NGAL autoantibody assay or in any other circumstance in which an assessment of NGAL levels or concentration might prove helpful: e.g., cancer-related assays (e.g., generally, or more specifically including but not limited to pancreatic cancer, breast cancer, ovarian/uterine cancer, leukemia, colon cancer, and brain cancer; see, e.g., U.S. Pat. App. Pub. No. 2007/0196876; see, also, U.S. Pat. Nos. 5,627,034 and 5,846,739); diagnosis of systemic inflammatory response syndrome (SIRS), sepsis, severe sepsis, septic shock and multiple organ dysfunction syndrome (MODS) (see, e.g., U.S. Pat. App. Pub. Nos. 2008/0050832 and 2007/0092911; see, also, U.S. Pat. No. 6,136,526); hematology applications (e.g., estimation of cell type); assessment of preeclampsia, obesity (metabolic syndrome), insulin resistance, hyperglycemia, tissue remodeling (when complexed with MMP-9; see, e.g., U.S. Pat. App. Pub. No. 2007/0105166 and U.S. Pat. No. 7,153,660), autoimmune diseases (e.g., rheumatoid arthritis, inflammatory bowel disease, multiple sclerosis), irritable bowel syndrome (see, e.g., U.S. Pat. App. Pub. Nos. 2008/0166719 and 2008/0085524), neurodegenerative disease, respiratory tract disease, inflammation, infection, periodontal disease (see, e.g., U.S. Pat. No. 5,866,432), and cardiovascular disease including venous thromboembolic disease (see, e.g., U.S. Pat. App. Pub. Nos. 2007/0269836), among others.

Moreover, any of the teachings of U.S. Provisional Application Nos. 60/981,470, 60/981,471 and 60/981,473, all filed on Oct. 19, 2007, and U.S. patent application Ser. Nos. 12/104,408, 12/104,410, and 12/104,413, all filed on Apr. 16, 2008, with respect to assay rare reagents NGAL antigen, anti-NGAL antibody, and an NGAL assay can be applied in the methods and kits as described herein and are each incorporated by reference in their entireties for their teachings regarding same.

Isolated Autoantibodies Reactive with Human NGAL or a Fragment Thereof

Also provided are isolated human NGAL autoantibodies. The isolated human NGAL autoantibodies can be IgG, IgA, or IgM antibodies. Preferably, the isolated autoantibodies are IgG antibodies. The autoantibodies can be obtained using routine techniques known in the art. For example, the autoantibodies can be obtained by separating such autoantibodies from their environment, such as, for example, from a mixture containing human NGAL autoantibodies. Such a mixture can be obtained from one or more subjects during the course of normal blood donation. Further, such a mixture can be obtained from one or more subjects receiving treatment with a human NGAL (or fragment or derivative thereof). Alternatively, the mixture can be obtained from one or more subjects with clinical signs of cardiovascular or renal disease or an endogenous concentration of NGAL that is higher than the clinically acceptable value for a normal population. Mixtures containing such autoantibodies can be obtained from freshly collected or stored blood, plasma or serum from such a subject and readily identified using the methods described herein. The autoantibodies can be isolated using routine immunoglobulin procedures, such as salt fractionation (e.g., ammonium sulfate precipitation), immunoprecipitation, affinity capture on a solid phase, gel electrophoresis, dialysis, or chromatography (e.g., protein A-sepharose chromatography, hydroxyapatite chromatography, affinity chromatography, anion-exchange chromatography, ion-exchange chromatography, immunoffinity chromatography, size exclusion chromatography, reversed-phase chromatography, etc.).

In particular, the specific binding partners described herein can be employed using standard techniques in the isolation of an autoantibody and/or the confirmation of the identification of the isolated autoantibody. The amino acid sequence of such an autoantibody can be determined using routine techniques known in the art, such as by automated Edman degradation. Alternatively, the amino acid sequence of such an antibody can be determined using mass spectrometry methods and techniques as described in Adamczyk et al., J. Immunol. Methods 260: 235-49 (2002); Adamczyk et al., Rapid Comm. Mass Spectrom. 14: 999-1007 (2000); Adamczyk et al., J. Immunol. Methods 237: 95-104 (2000); Adamczyk et al., Rapid Comm. Mass Spectrom. 14: 49-51 (2000); Adamczyk et al., Rapid Comm. Mass Spectrom. 13: 1813-1817 (1999); and Adamczyk et al., Rapid Comm. Mass Spectrom. 13: 1413-22 (1999).

Once the amino acid sequence of the autoantibody is determined, the nucleic acid sequence of the autoantibody also can be determined using routine techniques known in the art. The nucleic acid sequence thus obtained can be used to express the autoantibody or autoantibody fragments in human or non-human cell lines, or non-human subjects as well-known to those skilled in the art of antibody engineering.

Additionally, once the amino acid and nucleic acid sequences of the isolated NGAL autoantibody are obtained, the amino acid and nucleic acid sequences can be directly synthesized using various solid-phase techniques (see, e.g., Roberge et al., Science 269: 202-204 (1995)), and automated synthesis can be achieved, for example, using the ABI 43 1 A Peptide Synthesizer (Perkin Elmer) in accordance with the instructions provided by the manufacturer. Additionally, the amino acid sequence obtainable from the isolated autoantibody described herein can be altered during direct synthesis and/or combined using chemical methods with a sequence from other subunits, or any part thereof, to produce variant sequences and hence variant human NGAL autoantibodies.

The human NGAL autoantibodies described herein can be used for a variety of different purposes. For example, these isolated autoantibodies can be used in screening methods to identify agents that are useful in inhibiting the binding of human NGAL or fragment thereof to at least one human anti-NGAL autoantibody. Specifically, such screening methods would involve preparing a mixture comprising an isolated human NGAL autoantibody. After such a mixture is prepared, the method would involve adding to the mixture, either simultaneously or sequentially, and in any order, human NGAL or fragment thereof and at least one agent to be tested (such as a pharmaceutical composition, etc.). The final step would involve determining whether the agent being tested inhibits the binding of NGAL or fragment thereof to the human NGAL autoantibody. It is contemplated that such a method could be partially or fully automated to allow for the screening of a large number of agents at one time. Agents determined to inhibit the binding of NGAL or a fragment thereof to the human NGAL autoantibody would be selected for further study for use as a potential therapeutic agent in treating cardiovascular or renal disease in a human. Additionally, the isolated autoantibodies described herein also could be employed in pharmaceutical compositions that can be used to treat cardiovascular or renal disease. Such pharmaceutical compositions would contain the isolated autoantibodies described herein and one or more pharmaceutically acceptable excipients.

In view of the above, a method of isolating an autoantibody that reacts with NGAL or a fragment thereof is provided. The method comprises the steps of (i) contacting NGAL (or a fragment thereof) with a biological sample, which is known to contain an autoantibody that reacts with NGAL, wherein the NGAL is optionally immobilized on a solid phase before or after contact with the biological sample, (ii) isolating NGAL to which is bound the autoantibody, and (iii) isolating the autoantibody from the NGAL. Also provided is an isolated autoantibody that reacts with NGAL or a fragment thereof.

### Assay Kit

A kit for assaying a test sample for the presence, amount or concentration of at least one autoantibody that reacts with NGAL (or a fragment thereof) in a test sample is also provided. The kit comprises at least one component for assaying the test sample for at least one autoantibody that reacts with NGAL (or a fragment thereof) and instructions for assaying the test sample for at least one autoantibody that reacts with NGAL (or a fragment thereof). The at least one component for assaying the test sample for at least one autoantibody that reacts with NGAL (or a fragment thereof) includes a composition comprising NGAL (or a fragment thereof), which is optionally immobilized on a solid phase, and/or a composition comprising an antibody that can bind to the at least one autoantibody that reacts with NGAL (or a fragment thereof), wherein the antibody is optionally detectably labeled. The instructions can also contain instructions for generating a standard curve or a reference standard for purposes of quantifying the autoantibodies. Such instructions optionally can be in printed form or on CD, DVD, or other format of recorded media.

The kit can further comprise at least one component for assaying the test sample for NGAL (or a fragment thereof) and instructions for assaying the test sample for NGAL (or a fragment thereof). The at least one component for assaying the test sample for NGAL (or a fragment thereof) includes a composition comprising an antibody that can bind to NGAL (or a fragment thereof), wherein the antibody is optionally detectably labeled.

Alternatively or additionally, the kit can comprise a calibrator or control, e.g., isolated or purified, NGAL antibody, and/or at least one container (e.g., tube, microtiter plates or strips, which can be already coated with NGAL) for conducting the assay, and/or a buffer, such as an assay buffer or a wash buffer, either one of which can be provided as a concentrated solution, a substrate solution for the detectable label (e.g., an enzymatic label), or a stop solution. Preferably, the kit comprises all components, i.e., reagents, standards, buffers, diluents, etc., which are necessary to perform the assay.

Any antibodies, which are provided in the kit, such as antibodies specific for NGAL, can incorporate a detectable label, such as a fluorophore, radioactive moiety, enzyme, biotin/avidin label, chromophore, chemiluminescent label, or the like, or the kit may include reagents for labeling the antibodies or reagents for detecting the antibodies (e.g., detection antibodies) and/or for labeling the analytes or reagents for detecting the analyte. The antibodies, calibrators and/or controls can be provided in separate containers or pre-dispensed into an appropriate assay format, for example, into microtiter plates.

Optionally, the kit includes quality control components (for example, sensitivity panels, calibrators, and positive controls). Preparation of quality control reagents is well-known in the art and is described on insert sheets for a variety of immunodiagnostic products. Sensitivity panel members optionally are used to establish assay performance characteristics, and further optionally are useful indicators of the integrity of the immunoassay kit reagents, and the standardization of assays.

The kit can also optionally include other reagents required to conduct a diagnostic assay or facilitate quality control evaluations, such as buffers, salts, enzymes, enzyme co-factors, substrates, detection reagents, and the like. Other components, such as buffers and solutions for the isolation and/or treatment of a test sample (e.g., pretreatment reagents), also can be included in the kit. The kit can additionally include one or more other controls. One or more of the components of the kit can be lyophilized, in which case the kit can further comprise reagents suitable for the reconstitution of the lyophilized components.

The various components of the kit optionally are provided in suitable containers as necessary, e.g., a microtiter plate. The kit can further include containers for holding or storing a sample (e.g., a container or cartridge for a urine sample). Where appropriate, the kit optionally also can contain reaction vessels, mixing vessels, and other components that facilitate the preparation of reagents or the test sample. The kit can also include one or more instrument for assisting with obtaining a test sample, such as a syringe, pipette, forceps, measured spoon, or the like.

Preferably, the detectable label is at least one acridinium compound as described herein. The kit can comprise at least one acridinium-9-carboxamide, at least one acridinium-9-carboxylate aryl ester, or any combinations thereof. If the detectable label is at least one acridinium compound, the kit also can comprise a source of hydrogen peroxide, such as a buffer, solution, a composition comprising at least one hydrogen peroxide-generating enzyme, or another means of generating hydrogen peroxide in situ, and/or at least one basic solution. If desired, the kit can contain a solid phase, such as a magnetic particle, bead, test tube, microtiter plate, cuvette, membrane, scaffolding molecule, film, filter paper, disc or chip.

### Adaptation of Method and Assay Kit

The kit (or components thereof), as well as the method of determining the concentration of NGAL autoantibody in a test sample by an assay as described below, can be adapted for use in a variety of automated and semi-automated systems (including those wherein the solid phase comprises a microparticle), as described, e.g., in U.S. Pat. Nos. 5,089,424 and 5,006,309, and as commercially marketed, e.g., by Abbott Laboratories (Abbott Park, Ill.) as ARCHITECT®.

Some of the differences between an automated or semi-automated system as compared to a non-automated system (e.g., ELISA) include the substrate to which the first specific binding partner (e.g., NGAL) is attached (which can impact sandwich formation and analyte reactivity), and the length and timing of the capture, detection and/or any optional wash steps. Whereas a non-automated format such as an ELISA may require a relatively longer incubation time with sample and capture reagent (e.g., about 2 hours) an automated or semi-automated format (e.g., ARCHITECT®, Abbott Laboratories) may have a relatively shorter incubation time (e.g., approximately 18 minutes for ARCHITECT®). Similarly, whereas a non-automated format such as an ELISA may incubate a detection antibody such as the conjugate reagent for a relatively longer incubation time (e.g., about 2 hours), an automated or semi-automated format (e.g., ARCHITECT®) may have a relatively shorter incubation time (e.g., approximately 4 minutes for the ARCHITECT®).

Other platforms available from Abbott Laboratories include, but are not limited to, AxSYM®, IMx® (see, e.g., U.S. Pat. No. 5,294,404, which is hereby incorporated by reference in its entirety), PRISM®, EIA (bead), and Quantum™ II, as well as other platforms. Additionally, the assays, kits and kit components can be employed in other formats, for example, on electrochemical or other hand-held or point-of-care assay systems. The present disclosure is, for example, applicable to the commercial Abbott Point of Care (i-STAT®, Abbott Laboratories) electrochemical immunoassay system that performs sandwich immunoassays. Immunosensors and their methods of manufacture and operation in single-use test devices are described, for example in, U.S. Pat. No. 5,063,081, U.S. Pat. App. Pub. No. 2003/0170881, U.S. Pat. App. Pub. No. 2004/0018577, U.S. Pat. App. Pub. No. 2005/0054078, and U.S. Pat. App. Pub. No. 2006/0160164, which are incorporated in their entireties by reference for their teachings regarding same.

In particular, with regard to the adaptation of an autoantibody assay to the I-STAT® system, the following configuration is preferred. A microfabricated silicon chip is manufactured with a pair of gold amperometric working electrodes and a silver-silver chloride reference electrode. On one of the working electrodes, polystyrene beads (0.2 mm diameter) with immobilized capture antibody are adhered to a polymer coating of patterned polyvinyl alcohol over the electrode. This chip is assembled into an I-STAT® cartridge with a fluidics format suitable for immunoassay. On a portion of the wall of the sample-holding chamber of the cartridge there is a layer comprising the second detection antibody labeled with alkaline phosphatase (or other label). Within the fluid pouch of the cartridge is an aqueous reagent that includes p-aminophenol phosphate.

In operation, a sample suspected of containing NGAL autoantibodies is added to the holding chamber of the test cartridge and the cartridge is inserted into the I-STAT® reader. After the second antibody (detection antibody) has dissolved into the sample, a pump element within the cartridge forces the sample into a conduit containing the chip. Here it is oscillated to promote formation of the sandwich between NGAL, NGAL autoantibody, and the labeled detection antibody. In the penultimate step of the assay, fluid is forced out of the pouch and into the conduit to wash the sample off the chip and into a waste chamber. In the final step of the assay, the alkaline phosphatase label reacts with p-aminophenol phosphate to cleave the phosphate group and permit the liberated p-aminophenol to be electrochemically oxidized at the working electrode. Based on the measured current, the reader is able to calculate the amount of NGAL autoantibody in the sample by means of an embedded algorithm and factory-determined calibration curve.

It further goes without saying that the methods and kits as described herein necessarily encompass other reagents and methods for carrying out the immunoassay. For instance, encompassed are various buffers such as are known in the art and/or which can be readily prepared or optimized to be employed, e.g., for washing, as a conjugate diluent, and/or as a calibrator diluent. An exemplary conjugate diluent is ARCHITECT® conjugate diluent employed in certain kits (Abbott Laboratories, Abbott Park, Ill.) and containing 2-(N-morpholino)ethanesulfonic acid (MES), a salt, a protein blocker, an antimicrobial agent, and a detergent. An exemplary calibrator diluent is ARCHITECT® human calibrator diluent employed in certain kits (Abbott Laboratories, Abbott Park, Ill.), which comprises a buffer containing MES, other salt, a protein blocker, and an antimicrobial agent.

### EXAMPLES

The following examples serve to illustrate the present disclosure. The examples are not intended to limit the scope of the claimed invention in any way.

### Example 1

This example describes the preparation and characterization of microplates coated with neutrophil gelatinase-associated lipocalin (NGAL).

Recombinant NGAL (R&D Systems, Minneapolis, Minn.) was dissolved in phosphate buffer (0.2 M, pH 8.0) to a concentration of 4 μg/mL. Microplates (Costar, Corning Life Sciences, Lowell, Mass.) were coated with the NGAL solution (100 μL/well) for two hours at 38° C. with mixing at 280 rpm. The plates were drained, and the NGAL solution was replaced with a solution of heat-inactivated bovine serum albumin (BSA, 2% w/v in phosphate-buffered saline (PBS), 300 μL/well). The plates were incubated at 38° C. for 1 hour with mixing at 280 rpm, and then drained. The plates were then washed (3×) with a solution of sucrose (2% w/v in PBS, 300 μL/well), drained, and dried under a stream of dry nitrogen.

A murine anti-NGAL antibody (R&D Systems, 100 ng/mL) labeled with an acridinium-9-carboxamide was added to an NGAL-coated microplate (100 μL/well), which was subsequently incubated for 1 hour at 37° C. and then washed with ARCHITECT® wash buffer (6×, 350 μL). The microplate was loaded into a Mithras microplate reader (Berthold Technologies Inc, Oak Ridge, Tenn.) equilibrated at 28° C. The chemiluminescence signal from each well was recorded for 2 seconds after the sequential addition of ARCHITECT® pre-trigger solution (100 μL) and ARCHITECT® trigger solution (100 μL). The NGAL-reactive murine monoclonal antibody conjugate bound specifically to the NGAL-coated wells, with an average signal of 1,763,268 relative light units (RLU) and a 1.2% (CV).

### Example 2

This example describes the analysis of test samples of human plasma for autoantibodies that react with NGAL.

Frozen normal donor plasma samples were obtained from the Abbott Laboratories (Abbott Park, Ill.) specimen bank and thawed at 2-8° C. prior to use. Microplates were prepared as described in Example 1. A murine anti-human IgG (subtype IgG2b, kappa) was labeled with a chemiluminescent acridinium-9-carboxamide. This antibody recognized all human IgG subtypes while having no significant reactivity toward human IgM or IgA, or rabbit, sheep or goat IgG. The sample (5 μL) was diluted with AxSYM® Troponin-I ADV preincubation diluent (95 μL) in the microplate well. After incubating at 37° C. for 2 hours, the plate was washed with ARCHITECT® wash buffer (6×, 350 μL). The murine anti-human IgG-specific monoclonal antibody-acridinium conjugate (100 μL) was then added, and the plate was incubated at 37° C. for 1 hour, before a final wash with ARCHITECT® wash buffer (6×, 350 μL). The microplate was loaded into a Mithras microplate reader (Berthold Technologies Inc, Oak Ridge, Tenn.) equilibrated at 28° C. The chemiluminescence signal from each well was recorded for 2 seconds after the sequential addition of ARCHITECT® pre-trigger solution (100 μL) and ARCHITECT® trigger solution (100 μL). General statistics for the population tested are shown in Table 2 and 3.

TABLE 2

| Variable | RLU_{Max} |
|---|---|
| Sample size | 192 |
| Lowest value | 460.0000 |
| Highest value | 21,1820.0000 |
| Arithmetic mean | 3,418.6979 |
| 95% CI for the mean | 1,163.6268 to 5,673.7691 |
| Median | 1,420.0000 |
| 95% CI for the median | 1,290.1393 to 1,520.0000 |
| Variance | 250,959,961.1229 |
| Standard deviation | 15,841.7159 |
| Relative standard deviation | 4.6338 (463.38%) |
| Standard error of the mean | 1,143.2774 |
| Coefficient of Skewness | 12.2033 (P < 0.0001) |
| Coefficient of Kurtosis | 159.1313 (P < 0.0001) |
| D′Agostino-Pearson test | reject Normality (P < 0.0001) |
| for Normal distribution | |

TABLE 3

| Percentile | RLU_{Max} | 95% Confidence Interval |
|---|---|---|
| 2.5 | 602.0000 | 481.6580 to 682.0077 |
| 25 | 1,015.0000 | 930.0000 to 1,130.0000 |
| 75 | 1,900.0000 | 1,740.5018 to 2,160.2879 |
| 90 | 2,708.0000 | 2,306.8791 to 4,520.2298 |
| 97.5 | 2,0188.0000 | 4,339.8081 to 121,955.6993 |

Generation of a box-and-whisker plot for 192 samples, in which the difference between the 25^{th} percentile and the 75^{th} percentile was 885.0, revealed that the outside value was ≧3,227.5 (17 outside; 8.9%) and the far outside value was ≧4,555.0 (11 outside; 5.7%). These results confirm that, surprisingly and unexpectedly, one or more autoantibodies reactive with NGAL are found in human samples.

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference.

The invention illustratively described herein may be suitably practiced in the absence of any element(s) or limitation(s), which is/are not specifically disclosed herein. Thus, for example, each instance herein of any of the terms “comprising,” “consisting essentially of,” and “consisting of” may be replaced with either of the other two terms. Likewise, the singular forms “a,” “an,” and “the” include plural references unless the context clearly dictates otherwise. Thus, for example, references to “the method” includes one or more methods and/or steps of the type, which are described herein and/or which will become apparent to those ordinarily skilled in the art upon reading the disclosure.

The terms and expressions, which have been employed, are used as terms of description and not of limitation. In this regard, where certain terms are defined under “Definitions” and are otherwise defined, described, or discussed elsewhere in the “Detailed Description,” all such definitions, descriptions, and discussions are intended to be attributed to such terms. There also is no intention in the use of such terms and expressions of excluding any equivalents of the features shown and described or portions thereof. Furthermore, while subheadings, e.g., “Definitions,” are used in the “Detailed Description,” such use is solely for ease of reference and is not intended to limit any disclosure made in one section to that section only; rather, any disclosure made under one subheading is intended to constitute a disclosure under each and every other subheading.

It is recognized that various modifications are possible within the scope of the claimed invention. Thus, it should be understood that, although the present invention has been specifically disclosed in the context of preferred embodiments and optional features, those skilled in the art may resort to modifications and variations of the concepts disclosed herein. Such modifications and variations are considered to be within the scope of the invention as defined by the appended claims.

What is claimed is:

1. A method of determining the presence, amount or concentration of at least one autoantibody that reacts with neutrophil gelatinase-associated lipocalin (NGAL) or a fragment thereof in a test sample, which method comprises assaying the test sample for at least one autoantibody that reacts with NGAL (or a fragment thereof) by an assay employing NGAL (or a fragment thereof) and at least one detectable label and comprising comparing a signal generated by the detectable label as a direct or indirect indication of the presence, amount or concentration of at least one autoantibody that reacts with NGAL (or a fragment thereof) in the test sample to a signal generated as a direct or indirect indication of the presence, amount or concentration of an antibody that reacts with NGAL (or a fragment thereof) in a control or calibrator, which is optionally part of a series of calibrators in which each of the calibrators differs from the other calibrators in the series by the concentration of an antibody that reacts with NGAL (or a fragment thereof), whereupon the presence, amount or concentration of at least one autoantibody that reacts with NGAL (or a fragment thereof) in the test sample is determined.

2. The method of claim 1, wherein the method comprises the following steps:
(i) contacting the test sample with NGAL (or a fragment thereof), which comprises a detectable label and binds to at least one autoantibody to form an NGAL (or a fragment thereof)/autoantibody complex, and
(ii) determining the presence, amount or concentration of at least one autoantibody, which reacts with NGAL (or a fragment thereof), in the test sample by detecting or measuring the signal generated by the detectable label in the NGAL (or fragment thereof)/autoantibody complex formed in (i), whereupon the presence, amount or concentration of at least one autoantibody that reacts with NGAL (or a fragment thereof) in the test sample is determined.

3. The method of claim 1, wherein the method comprises the following steps:
(i) contacting the test sample with NGAL (or a fragment thereof), which binds to at least one autoantibody and which is optionally immobilized on a solid phase, so as to form an NGAL (or a fragment thereof)/autoantibody complex,
(ii) contacting the NGAL (or a fragment thereof)/autoantibody complex with at least one detection antibody, which comprises a detectable label and binds to the autoantibody to form an NGAL (or a fragment thereof)/autoantibody/detection antibody complex, and
(iii) determining the presence, amount or concentration of an autoantibody, which reacts with NGAL (or fragment thereof), in the test sample by detecting or measuring the signal generated by the detectable label in the NGAL (or a fragment thereof)/autoantibody/detection antibody complex formed in (ii), whereupon the presence, amount or concentration of an autoantibody that reacts with NGAL (or fragment thereof) in the test sample is determined,
wherein the method optionally further comprises removing any unbound at least one autoantibody after step (i) and removing any unbound at least one detection antibody after step (ii).

4. The method of claim 1, wherein the detectable label is an acridinium compound.

5. The method of claim 4, wherein the acridinium compound is an acridinium-9-carboxamide or an acridinium-9-carboxylate aryl ester.

6. The method of claim 2, wherein the detectable label is an acridinium compound.

7. The method of claim 6, wherein the acridinium compound is an acridinium-9-carboxamide or an acridinium-9-carboxylate aryl ester.

8. The method of claim 3, wherein the detectable label is an acridinium compound.

9. The method of claim 8, wherein the acridinium compound is an acridinium-9-carboxamide or an acridinium-9-carboxylate aryl ester.

10. The method of claim 1, which further comprises previously, simultaneously or subsequently determining the concentration of NGAL (or a fragment thereof) in the test sample, which method comprises assaying the test sample for NGAL (or a fragment thereof) by an assay employing at least one specific binding partner for NGAL (or a fragment thereof) and at least one detectable label and comprising comparing a signal generated by the detectable label as a direct or indirect indication of the concentration of NGAL (or a fragment thereof) in the test sample to a signal generated as a direct or indirect indication of the concentration of NGAL in a control or calibrator, which is optionally part of a series of calibrators in which each of the calibrators differs from the other calibrators in the series by the concentration of NGAL, whereupon the concentration of NGAL (or a fragment thereof) in the test sample is determined.

11. The method of claim 2, which further comprises previously, simultaneously or subsequently determining the concentration of NGAL (or a fragment thereof) in the test sample, which method comprises assaying the test sample for NGAL (or a fragment thereof) by an assay employing at least one specific binding partner for NGAL (or a fragment thereof) and at least one detectable label and comprising comparing a signal generated by the detectable label as a direct or indirect indication of the concentration of NGAL (or a fragment thereof) in the test sample to a signal generated as a direct or indirect indication of the concentration of NGAL in a control or calibrator, which is optionally part of a series of calibrators in which each of the calibrators differs from the other calibrators in the series by the concentration of NGAL, whereupon the concentration of NGAL (or a fragment thereof) in the test sample is determined.

12. The method of claim 3, which further comprises previously, simultaneously or subsequently determining the concentration of NGAL (or a fragment thereof) in the test sample, which method comprises assaying the test sample for NGAL (or a fragment thereof) by an assay employing at least one specific binding partner for NGAL (or a fragment thereof) and at least one detectable label and comprising comparing a signal generated by the detectable label as a direct or indirect indication of the concentration of NGAL (or a fragment thereof) in the test sample to a signal generated as a direct or indirect indication of the concentration of NGAL in a control or calibrator, which is optionally part of a series of calibrators in which each of the calibrators differs from the other calibrators in the series by the concentration of NGAL, whereupon the concentration of NGAL (or a fragment thereof) in the test sample is determined.

13. The method of claim 1, which further comprises diagnosing, prognosticating, or assessing the efficacy of a therapeutic/prophylactic treatment of a patient from whom the test sample was obtained, wherein, if the method further comprises assessing the efficacy of a therapeutic/prophylactic treatment of the patient from whom the test sample was obtained, the method optionally further comprises modifying the therapeutic/prophylactic treatment of the patient as needed to improve efficacy.

14. The method of claim 2, which further comprises diagnosing, prognosticating, or assessing the efficacy of a therapeutic/prophylactic treatment of a patient from whom the test sample was obtained, wherein, if the method further comprises assessing the efficacy of a therapeutic/prophylactic treatment of the patient from whom the test sample was obtained, the method optionally further comprises modifying the therapeutic/prophylactic treatment of the patient as needed to improve efficacy.

15. The method of claim 3, which further comprises diagnosing, prognosticating, or assessing the efficacy of a therapeutic/prophylactic treatment of a patient from whom the test sample was obtained, wherein, if the method further comprises assessing the efficacy of a therapeutic/prophylactic treatment of the patient from whom the test sample was obtained, the method optionally further comprises modifying the therapeutic/prophylactic treatment of the patient as needed to improve efficacy.

16. The method of claim 10, which further comprises diagnosing, prognosticating, or assessing the efficacy of a therapeutic/prophylactic treatment of a patient
from whom the test sample was obtained, wherein, if the method further comprises assessing the efficacy of a therapeutic/prophylactic treatment of the patient from whom the test sample was obtained, the method optionally further comprises modifying the therapeutic/prophylactic treatment of the patient as needed to improve efficacy.

17. The method of claim 11, which further comprises diagnosing, prognosticating, or assessing the efficacy of a therapeutic/prophylactic treatment of a patient from whom the test sample was obtained, wherein, if the method further comprises assessing the efficacy of a therapeutic/prophylactic treatment of the patient from whom the test sample was obtained, the method optionally further comprises modifying the therapeutic/prophylactic treatment of the patient as needed to improve efficacy.

18. The method of claim 12, which further comprises diagnosing, prognosticating, or assessing the efficacy of a therapeutic/prophylactic treatment of a patient from whom the test sample was obtained, wherein, if the method further comprises assessing the efficacy of a therapeutic/prophylactic treatment of the patient from whom the test sample was obtained, the method optionally further comprises modifying the therapeutic/prophylactic treatment of the patient as needed to improve efficacy.

19. The method of claim 1, wherein the method is adapted for use in an automated system or a semi-automated system.

20. The method of claim 2, wherein the method is adapted for use in an automated system or a semi-automated system.

21. The method of claim 3, wherein the method is adapted for use in an automated system or a semi-automated system.

22. The method of claim 10, wherein the method is adapted for use in an automated system or a semi-automated system.

23. The method of claim 11, wherein the method is adapted for use in an automated system or a semi-automated system.

24. The method of claim 12, wherein the method is adapted for use in an automated system or a semi-automated system.

25. A kit for assaying a test sample for the presence, amount or concentration of at least one autoantibody that reacts with NGAL (or a fragment thereof) in a test sample, which kit comprises at least one component for assaying the test sample for at least one autoantibody that reacts with NGAL (or a fragment thereof) and instructions for assaying the test sample for at least one autoantibody that reacts with NGAL (or a fragment thereof), wherein the at least one component for assaying the test sample for at least one autoantibody that reacts with NGAL (or a fragment thereof) includes a composition comprising NGAL (or a fragment thereof), which is optionally immobilized on a solid phase, and/or a composition comprising an antibody that can bind to the at least one autoantibody that reacts with NGAL (or a fragment thereof), wherein the NGAL (or a fragment thereof) or the antibody is optionally detectably labeled.

26. The kit of claim 25, which further comprises at least one component for assaying the test sample for NGAL (or a fragment thereof) and instructions for assaying the test sample for NGAL (or a fragment thereof), wherein the at least one component for assaying the test sample for NGAL (or a fragment thereof) includes a composition comprising a specific binding partner for NGAL (or a fragment thereof), wherein the specific binding partner for NGAL is optionally detectably labeled.

27. A method of isolating an autoantibody that reacts with NGAL, which method comprises the steps of:
(i) contacting NGAL (or a fragment thereof) with a biological sample, which is known to contain an autoantibody that reacts with NGAL (or a fragment thereof), wherein the NGAL (or a fragment thereof) is optionally immobilized on a solid phase before or after contact with the biological sample,
(ii) isolating NGAL (or a fragment thereof) to which is bound the autoantibody, and
(iii) isolating the autoantibody from the NGAL (or a fragment thereof),
whereupon an autoantibody that reacts with NGAL (or a fragment thereof) is isolated.

28. An isolated autoantibody that reacts with NGAL or a fragment thereof.

29. A method for determining the reliability of a result obtained from a separate assay that was previously, simultaneously or subsequently performed for detecting or quantifying the amount or concentration of NGAL in a test sample obtained from a subject, wherein the method comprises:
(a) determining the amount or the concentration in the test sample of at least one autoantibody that reacts with NGAL; and
(b) comparing the amount or the concentration in step (a) to a predetermined level, wherein if the amount or the concentration is elevated as compared to a predetermined level, then the amount or concentration of NGAL as determined by separate assay is considered not to be reliable, and wherein if the amount or the concentration in step (a) is lower or the same as a predetermined level, then the amount or concentration of NGAL as determined by separate assay is considered to be reliable.


SEQUENCE LISTING (3 sequences)

SEQ ID NO: 1 - protein, 198 aa, Homo sapiens
  MISC_FEATURE (1)..(20): The first 20 amino acids are the signal peptide which would be labeled 1 to 20, with Gln (at position 21) being considered the first amino acid of the mature NGAL peptide
MPLGLLWLGLALLGALHAQAQDSTSDLIPAPPLSKVPLQQNFQDNQFQGKWYVVGLAGNAILREDKDPQKMYATIYELKEDKSYNVTSVLFRKKKCDYWIRTFVPGCQPGEFTLGNIKSYPGLTSYLVRVVSTNYNQHAMVFFKKVSQNREYFKITLYGRTKELTSELKENFIRFSKSLGLPENHIVFPVPIDQCIDG

SEQ ID NO: 2 - protein, 178 aa, Artificial sequence
  Chemically synthesized
QDSTSDLIPAPPLSKVPLQQNFQDNQFQGKWYVVGLAGNAILREDKDPQKMYATIYELKEDKSYNVTSVLFRKKKCDYWIRTFVPGCQPGEFTLGNIKSYPGLTSYLVRVVSTNYNQHAMVFFKKVSQNREYFKITLYGRTKELTSELKENFIRFSKSLGLPENHIVFPVPIDQCIDG

SEQ ID NO: 3 - DNA, 612 bp, Homo sapiens
atgcccctaggtctcctgtggctgggcctagccctgttgggggctctgcatgcccaggcccaggactccacctcagacctgatcccagccccacctctgagcaaggtccctctgcagcagaacttccaggacaaccaattccaggggaagtggtatgtggtaggcctggcagggaatgcaattctcagagaagacaaagacccgcaaaagatgtatgccaccatctatgagctgaaagaagacaagagctacaatgtcacctccgtcctgtttaggaaaaagaagtgtgactactggatcaggacttttgttccaggttgccagcccggcgagttcacgctgggcaacattaagagttaccctggattaacgagttacctcgtccgagtggtgagcaccaactacaaccagcatgctatggtgttcttcaagaaagtttctcaaaacagggagtacttcaagatcaccctctacgggagaaccaaggagctgacttcggaactaaaggagaacttcatccgcttctccaaatctctgggcctccctgaaaaccacatcgtcttccctgtcccaatcgaccagtgtatcgacggccatcatcaccatcaccat